SEQUENCE LISTING (69 sequences)

SEQ ID NO: 1 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 3 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 550 aa, Homo sapiens
MPPPRTGRGLLWLGLVLSSVCVALGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 5 - protein, 517 aa, Homo sapiens
TDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 6 - protein, 525 aa, synthetic construct
  VARIANT 59: note = cysteine or formylglycine
SETQANSTTDALNVLLIIVDDLRPSLGCYGDKLVRSPNIDQLASHSLLFQNAFAQQAVXAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 7 - protein, 525 aa, Homo sapiens
SETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 8 - protein, 525 aa, synthetic construct
  SITE 59: note = formylglycine
SETQANSTTDALNVLLIIVDDLRPSLGCYGDKLVRSPNIDQLASHSLLFQNAFAQQAVGAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 9 - protein, 15 aa, Homo sapiens
EPKSCDKTHTCPPCP

SEQ ID NO: 10 - protein, 10 aa, Homo sapiens
DKTHTCPPCP

SEQ ID NO: 11 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWSSYKTTPPVLDSDGSFFLYSKLTVTKEEWQQGFVFSCSVMHEALHNHYTQKSLSLSPGK

SEQ ID NO: 12 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWSSYKTTPPVLDSDGSFFLYSKLTVTKEEWQQGFVFSCSVMHEALHNHYTQKSLSLSPGK

SEQ ID NO: 13 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWASYKTTPPVLDSDGSFFLYSKLTVTKEEWQQGFVFSCSVMHEALHNHYTQKSLSLSPGK

SEQ ID NO: 14 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWANYKTTPPVLDSDGSFFLYSKLTVTKEEWQQGFVFSCSVMHEALHNHYTQKSLSLSPGK

SEQ ID NO: 15 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVWWESYGTEWSSYKTTPPVLDSDGSFFLYSKLTVTKEEWQQGFVFSCSVMHEALHNHYTQKSLSLSPGK

SEQ ID NO: 16 - protein, 217 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVWWESYGTEWSSYKTTPPVLDSDGSFFLYSKLTVTKEEWQQGFVFSCSVMHEALHNHYTQKSLSLSPGK

SEQ ID NO: 17 - protein, 217 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVWWESYGTEWSSYKTTPPVLDSDGSFFLYSKLTVTKEEWQQGFVFSCSVMHEALHNHYTQKSLSLSPGK

SEQ ID NO: 18 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVWWESYGTEWSSYKTTPPVLDSDGSFFLYSKLTVTKEEWQQGFVFSCSVMHEALHNHYTQKSLSLSPGK

SEQ ID NO: 19 - protein, 217 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWSSYKTTPPVLDSDGSFFLYSKLTVTKEEWQQGFVFSCSVMHEALHNHYTQKSLSLSPGK

SEQ ID NO: 20 - protein, 217 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVLWESYGTEWSSYKTTPPVLDSDGSFFLYSKLTVTKEEWQQGFVFSCSVMHEALHNHYTQKSLSLSPGK

SEQ ID NO: 21 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWSSYKTTPPVLDSDGSFFLYSKLTVTKEEWQQGFVFSCSVMHEALHNHYTQKSLSLSPGK

SEQ ID NO: 22 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPGK

SEQ ID NO: 23 - protein, 217 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPGK

SEQ ID NO: 24 - protein, 217 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPGK

SEQ ID NO: 25 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPGK

SEQ ID NO: 26 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 27 - protein, 217 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 28 - protein, 217 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 29 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 30 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 31 - protein, 217 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 32 - protein, 757 aa, synthetic construct
  VARIANT 59: note = cysteine or formylglycine
SETQANSTTDALNVLLIIVDDLRPSLGCYGDKLVRSPNIDQLASHSLLFQNAFAQQAVXAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMPGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 33 - protein, 757 aa, synthetic construct
SETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMPGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 34 - protein, 757 aa, synthetic construct
  SITE 59: note = formylglycine
SETQANSTTDALNVLLIIVDDLRPSLGCYGDKLVRSPNIDQLASHSLLFQNAFAQQAVGAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMPGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 35 - protein, 757 aa, synthetic construct
  VARIANT 59: note = cysteine or formylglycine
SETQANSTTDALNVLLIIVDDLRPSLGCYGDKLVRSPNIDQLASHSLLFQNAFAQQAVXAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 36 - protein, 757 aa, synthetic construct
SETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 37 - protein, 757 aa, synthetic construct
  SITE 59: note = formylglycine
SETQANSTTDALNVLLIIVDDLRPSLGCYGDKLVRSPNIDQLASHSLLFQNAFAQQAVGAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 38 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 39 - protein, 181 aa, Homo sapiens
NSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVS

SEQ ID NO: 40 - protein, 5 aa, synthetic construct
GGGGS

SEQ ID NO: 41 - protein, 10 aa, synthetic construct
GGGGSGGGGS

SEQ ID NO: 42 - protein, 216 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 43 - protein, 216 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWSSYKTTPPVLDSDGSFFLYSKLTVTKEEWQQGFVFSCSVMHEALHNHYTQKSLSLSPG

SEQ ID NO: 44 - protein, 216 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVWWESYGTEWSSYKTTPPVLDSDGSFFLYSKLTVTKEEWQQGFVFSCSVMHEALHNHYTQKSLSLSPG

SEQ ID NO: 45 - protein, 216 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVWWESYGTEWSSYKTTPPVLDSDGSFFLYSKLTVTKEEWQQGFVFSCSVMHEALHNHYTQKSLSLSPG

SEQ ID NO: 46 - protein, 216 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVWWESYGTEWSSYKTTPPVLDSDGSFFLYSKLTVTKEEWQQGFVFSCSVMHEALHNHYTQKSLSLSPG

SEQ ID NO: 47 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVWWESYGTEWSSYKTTPPVLDSDGSFFLYSKLTVTKEEWQQGFVFSCSVMHEALHNHYTQKSLSLSPG

SEQ ID NO: 48 - protein, 216 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWSSYKTTPPVLDSDGSFFLYSKLTVTKEEWQQGFVFSCSVMHEALHNHYTQKSLSLSPG

SEQ ID NO: 49 - protein, 216 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWSSYKTTPPVLDSDGSFFLYSKLTVTKEEWQQGFVFSCSVMHEALHNHYTQKSLSLSPG

SEQ ID NO: 50 - protein, 216 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVLWESYGTEWSSYKTTPPVLDSDGSFFLYSKLTVTKEEWQQGFVFSCSVMHEALHNHYTQKSLSLSPG

SEQ ID NO: 51 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWSSYKTTPPVLDSDGSFFLYSKLTVTKEEWQQGFVFSCSVMHEALHNHYTQKSLSLSPG

SEQ ID NO: 52 - protein, 216 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWANYKTTPPVLDSDGSFFLYSKLTVTKEEWQQGFVFSCSVMHEALHNHYTQKSLSLSPG

SEQ ID NO: 53 - protein, 216 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 54 - protein, 216 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 55 - protein, 216 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 56 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 57 - protein, 216 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWASYKTTPPVLDSDGSFFLYSKLTVTKEEWQQGFVFSCSVMHEALHNHYTQKSLSLSPG

SEQ ID NO: 58 - protein, 216 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPG

SEQ ID NO: 59 - protein, 216 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPG

SEQ ID NO: 60 - protein, 216 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPG

SEQ ID NO: 61 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPG

SEQ ID NO: 62 - protein, 216 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 63 - protein, 216 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 64 - protein, 756 aa, synthetic construct
  VARIANT 59: note = cysteine or formylglycine
SETQANSTTDALNVLLIIVDDLRPSLGCYGDKLVRSPNIDQLASHSLLFQNAFAQQAVXAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMPGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 65 - protein, 756 aa, synthetic construct
SETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMPGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 66 - protein, 756 aa, synthetic construct
  SITE 59: note = formylglycine
SETQANSTTDALNVLLIIVDDLRPSLGCYGDKLVRSPNIDQLASHSLLFQNAFAQQAVGAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMPGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 67 - protein, 756 aa, synthetic construct
  VARIANT 59: note = cysteine or formylglycine
SETQANSTTDALNVLLIIVDDLRPSLGCYGDKLVRSPNIDQLASHSLLFQNAFAQQAVXAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 68 - protein, 756 aa, synthetic construct
SETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 69 - protein, 756 aa, synthetic construct
  SITE 59: note = formylglycine
SETQANSTTDALNVLLIIVDDLRPSLGCYGDKLVRSPNIDQLASHSLLFQNAFAQQAVGAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

(19) United States
(12) Patent Application Publication
(10) Pub. No.: US 2025/0018015 A1
(43) Pub. Date: Jan. 16, 2025

(54) METHODS FOR THE TREATMENT OF HUNTER SYNDROME

(71) Applicant: DENALI THERAPEUTICS INC., South San Francisco, CA (US)

(72) Inventors: Annie ARGUELLO, San Francisco, CA (US); Akhil BHALLA, San Francisco, CA (US); Jeffrey Mark HARRIS, San Francisco, CA (US); Matthew D. TROYER, Piedmont, CA (US)

(73) Assignee: DENALI THERAPEUTICS INC., South San Francisco, CA (US)

(21) Appl. No.: 18/290,585

(22) PCT Filed: Jul. 22, 2022
(86) PCT No.: PCT/US2022/038077

§ 371 Date: Jan. 19, 2024

Related U.S. Application Data
(60) Provisional application No. 63/307,571, filed on Feb. 7, 2022.
(60) Provisional application No. 63/307,576, filed on Feb. 7, 2022.
(60) Provisional application No. 63/307,580, filed on Feb. 7, 2022.
(60) Provisional application No. 63/225,347, filed on Jul. 23, 2021.
(60) Provisional application No. 63/225,362, filed on Jul. 23, 2021.
(60) Provisional application No. 63/225,379, filed on Jul. 23, 2021.
(60) Provisional application No. 63/225,391, filed on Jul. 23, 2021.

(51) Int. Cl. A61K 38/46; A61K 38/17; A61P 3/00; G01N 33/68

(52) U.S. Cl. CPC A61K 38/465; A61K 38/177; A61P 3/00; G01N 33/6893; G01N 2333/4742; G01N 2333/916; G01N 2800/52

(57) ABSTRACT

Certain embodiments provide a method of providing added peripheral IDS activity to a subject with Hunter syndrome, comprising administering to the subject a therapeutically effective dose of a pharmaceutical composition comprising a protein comprising an IDS amino acid sequence and a TfR binding domain. The added peripheral IDS activity can be measured by comparing levels of keratan sulfate (KS) in the subject after administration of the pharmaceutical composition relative to a baseline level. Certain embodiments also provide methods of providing a clinical benefit to a subject with Hunter syndrome and improving treatment in a patient having non-neuronopathic Hunter syndrome, as well as methods and treatment regimens for resolving infusion related reactions (IRRs).

### CROSS-REFERENCE TO RELATED APPLICATIONS

This application claims priority to U.S. Provisional Application Ser. No. 63/225,347, filed Jul. 23, 2021, U.S. Provisional Application Ser. No. 63/225,379, filed Jul. 23, 2021, U.S. Provisional Application Ser. No. 63/225,362, filed Jul. 23, 2021, U.S. Provisional Application Ser. No. 63/225,391, filed Jul. 23, 2021, U.S. Provisional Application Ser. No. 63/307,571, filed Feb. 7, 2022, U.S. Provisional Application Ser. No. 63/307,580, filed Feb. 7, 2022, and U.S. Provisional Application Ser. No. 63/307,576, filed Feb. 7, 2022. The entire content of the applications referenced above are hereby incorporated by reference herein.

### BACKGROUND

Hunter syndrome, or MPS II, is a rare, X-linked recessive disorder caused by IDS gene mutations. Insufficient iduronate 2-sulfatase (IDS) activity leads to accumulation of the glycosaminoglycans (GAGs) heparan sulfate (HS) and dermatan sulfate (DS) and to lysosomal dysfunction in multiple organs and tissues. Approximately two-thirds of patients display a neuronopathic phenotype (nMPS II). A recombinant form of IDS has been approved to treat Hunter syndrome, but it has little effect on the brain due to difficulties in delivering the recombinant enzyme across the blood-brain barrier (BBB). Accordingly, there is a need for more effective therapies that treat both the peripheral and central nervous system (CNS) symptoms of Hunter syndrome.

### SUMMARY

Certain embodiments relate to a method of providing added peripheral iduronate 2-sulfatase (IDS) activity relative to a standard-of-care treatment to a subject with Hunter syndrome, comprising administering to the subject a therapeutically effective dose of a pharmaceutical composition comprising a protein, wherein the administration of the pharmaceutical composition reduces serum levels of keratan sulfate (KS) in the subject relative to a baseline level measured in the subject prior to the administration, and wherein the protein comprises an IDS amino acid sequence and a transferrin receptor (TfR) binding domain. In certain embodiments, the protein comprises:
a) a fusion polypeptide comprising a first Fc polypeptide linked to an iduronate 2-sulfatase (IDS) amino acid sequence, wherein the fusion polypeptide comprises SEQ ID NO:35, 36, 37, 67, 68, or 69; and
b) a second Fc polypeptide comprising SEQ ID NO:29 or 56.

In certain embodiments, the administration of the pharmaceutical composition reduces serum levels of KS in the subject to a baseline level measured in a healthy subject or in a subject that does not have Hunter syndrome.

Certain embodiments provide a pharmaceutical composition comprising a protein for use in a method of providing added peripheral IDS activity relative to a standard-of-care treatment to a subject with Hunter syndrome, the method comprising administering to the subject a therapeutically effective dose of the pharmaceutical composition, wherein the administration of the pharmaceutical composition reduces serum levels of KS in the subject relative to a baseline level measured in the subject prior to the administration, and wherein the protein comprises an IDS amino acid sequence and a TfR binding domain.

Certain embodiments provide the use of a protein in the preparation of medicament for providing added peripheral IDS activity relative to a standard-of-care treatment to a subject with Hunter syndrome by administering to the subject a therapeutically effective dose of the medicament, wherein the medicament is capable of reducing serum levels of KS in the subject relative to a baseline level measured in the subject prior to the administration, and wherein the protein comprises an IDS amino acid sequence and a TfR binding domain.

Certain embodiments also provide a method of reducing and/or normalizing serum KS levels in a subject with Hunter syndrome, comprising administering to the subject a therapeutically effective dose of a pharmaceutical composition comprising a protein, wherein the protein comprises an IDS amino acid sequence and a TfR binding domain. In certain embodiments, the protein comprises:
a) a fusion polypeptide comprising a first Fc polypeptide linked to an IDS amino acid sequence, wherein the fusion polypeptide comprises SEQ ID NO:35, 36, 37, 67, 68 or 69; and
b) a second Fc polypeptide comprising SEQ ID NO:29 or 56.

Certain embodiments provide a pharmaceutical composition comprising a protein for use in a method of reducing and/or normalizing serum KS levels in a subject with Hunter syndrome, the method comprising administering to the subject a therapeutically effective dose of the pharmaceutical composition, wherein the protein comprises an IDS amino acid sequence and a TfR binding domain.

Certain embodiments provide the use of a protein in the preparation of medicament for reducing and/or normalizing serum KS levels in a subject with Hunter syndrome by administering to the subject a therapeutically effective dose of the medicament, wherein the protein comprises an IDS amino acid sequence and a TfR binding domain.

Certain embodiments provide a method for evaluating peripheral IDS activity in a subject having Hunter syndrome, comprising measuring the level of KS in a sample (e.g., serum) from the subject obtained at a time point after administration of a pharmaceutical composition comprising a protein; wherein a decrease in KS levels in the sample obtained after administration of the pharmaceutical composition as compared to the levels of KS in a sample (e.g., serum) from the subject obtained prior to administration of the pharmaceutical composition indicates added peripheral IDS activity in the subject, and wherein the protein comprises an IDS amino acid sequence and a transferrin receptor (TfR) binding domain. In certain embodiments, such a subject is identified as a candidate for treatment. In certain embodiments, such a subject is identified as a candidate for adjusting a treatment regimen for the subject.

Certain embodiments provide a method for evaluating peripheral IDS activity in a subject having Hunter syndrome, comprising:
(a) determining a first marker profile by detecting the level of KS in a sample (e.g., serum) from the subject obtained at baseline or prior to initiation of treatment with a pharmaceutical composition comprising a protein;
(b) administering to the subject the pharmaceutical composition comprising the protein;
(c) determining a second marker profile by detecting the level of KS in a sample (e.g., serum) from the subject obtained at a time point after administration of the pharmaceutical composition comprising the protein; and
(d) comparing the first and the second marker profiles;
wherein a decrease in KS levels between the first and the second marker profiles indicates added peripheral IDS activity in the subject, and
wherein the protein comprises an IDS amino acid sequence and a transferrin receptor (TfR) binding domain. In certain embodiments, the protein comprises: (i) a fusion polypeptide comprising a first Fc polypeptide linked to an IDS amino acid sequence, wherein the fusion polypeptide comprises SEQ ID NO: 35, 36, 37, 67, 68 or 69; and (ii) a second Fc polypeptide comprising SEQ ID NO: 29 or 56.

Certain embodiments relate to a method of providing added peripheral IDS activity relative to a standard-of-care treatment to a subject with Hunter syndrome, comprising administering to the subject a therapeutically effective dose (e.g., at least 23 weekly therapeutically effective doses) of a pharmaceutical composition comprising a protein, wherein the administration of the pharmaceutical composition normalizes levels of urine heparan sulfate (HS) and/or dermatan sulfate (DS) in the subject by at least 12 weekly doses, and wherein normalization is sustained after at least 23 weekly doses, and wherein the protein comprises an IDS amino acid sequence and a TfR binding domain. In certain embodiments, the protein comprises: (i) a fusion polypeptide comprising a first Fc polypeptide linked to an IDS amino acid sequence, wherein the fusion polypeptide comprises SEQ ID NO: 35, 36, 37, 67, 68 or 69; and (ii) a second Fc polypeptide comprising SEQ ID NO: 29 or 56.

Certain embodiments provide a pharmaceutical composition comprising a protein for use in a method of providing added peripheral IDS activity relative to a standard-of-care treatment to a subject with Hunter syndrome, the method comprising administering to the subject a therapeutically effective dose (e.g., at least 23 weekly therapeutically effective doses) of the pharmaceutical composition, wherein the administration of the pharmaceutical composition normalizes levels of urine HS and/or DS in the subject by at least 12 weekly doses, wherein normalization is sustained after at least 23 weekly doses, and wherein the protein comprises an IDS amino acid sequence and a TfR binding domain.

Certain embodiments provide the use of a protein in the preparation of medicament for providing added peripheral IDS activity relative to a standard-of-care treatment to a subject with Hunter syndrome by administering to the subject a therapeutically effective dose (e.g., at least 23 weekly therapeutically effective doses) of the medicament, wherein the administration of the medicament normalizes levels of urine HS and/or DS in the subject by at least 12 weekly doses, and wherein normalization is sustained after at least 23 weekly doses, and wherein the protein comprises an IDS amino acid sequence and a TfR binding domain.

Certain embodiments provide a method of normalizing urine HS and/or DS levels in a subject with Hunter syndrome, comprising administering to the subject a therapeutically effective dose (e.g., at least 23 weekly therapeutically effective doses) of a pharmaceutical composition comprising a protein, wherein the HS and/or DS levels in the subject are normalized by at least 12 weekly doses, wherein normalization is sustained after at least 23 weekly doses, and wherein the protein comprises an IDS amino acid sequence and a TfR binding domain. In certain embodiments, the protein comprises: (i) a fusion polypeptide comprising a first Fc polypeptide linked to an IDS amino acid sequence, wherein the fusion polypeptide comprises SEQ ID NO: 35, 36, 37, 67, 68 or 69; and (ii) a second Fc polypeptide comprising SEQ ID NO: 29 or 56.

Certain embodiments provide a pharmaceutical composition comprising a protein for use in a method of normalizing urine HS and/or DS levels in a subject with Hunter syndrome, the method comprising administering to the subject a therapeutically effective dose (e.g., at least 23 weekly therapeutically effective doses) of the pharmaceutical composition, wherein the HS and/or DS levels in the subject are normalized by at least 12 weekly doses, wherein normalization is sustained after at least 23 weekly doses, and wherein the protein comprises an IDS amino acid sequence and a TfR binding domain.

Certain embodiments provide the use of a protein in the preparation of medicament for normalizing urine HS and/or DS levels in a subject with Hunter syndrome by administering to the subject a therapeutically effective dose (e.g., at least 23 weekly therapeutically effective doses) of the medicament, wherein the HS and/or DS levels in the subject are normalized by at least 12 weekly doses, wherein normalization is sustained after at least 23 weekly doses, and wherein the protein comprises an IDS amino acid sequence and a TfR binding domain.

Certain embodiments provide a method of providing a clinical benefit to a subject with Hunter syndrome, comprising administering to the subject a therapeutically effective dose of a pharmaceutical composition comprising a protein, wherein the administration of the pharmaceutical composition produces an improvement in a clinical outcome as measured by one or more clinical Global Impression of Change (instruments relative to baseline levels measured in the subject prior to the administration, and wherein the protein comprises:
a) a fusion polypeptide comprising a first Fc polypeptide linked to an iduronate 2-sulfatase (IDS) amino acid sequence, wherein the fusion polypeptide comprises SEQ ID NO:35, 36, 37, 67, 68, or 69; and
b) a second Fc polypeptide comprising SEQ ID NO:29 or 56.

In certain embodiments, the one or more clinical Global Impression of Change instruments are Clinician Global Impression of Change (CGI-C) and/or Parent/Caregiver Global Impression of Change (PGI-C). In certain other embodiments, the one or more clinical Global Impression of Change instruments are Clinician Global Impression of Change (CGI-C) and/or Caregiver Global Impression of Change (CaGI-C).

Certain embodiments also provide a pharmaceutical composition comprising a protein for use in a method of providing a clinical benefit to a subject with Hunter syndrome, the method comprising administering a therapeutically effective dose of the pharmaceutical composition to the subject, wherein the administration of the pharmaceutical composition produces an improvement in a clinical outcome as measured by one or more clinical Global Impression of Change instruments relative to baseline levels measured in the subject prior to the administration, and wherein the protein comprises:
a) a fusion polypeptide comprising a first Fc polypeptide linked to an iduronate 2-sulfatase (IDS) amino acid sequence, wherein the fusion polypeptide comprises SEQ ID NO:35, 36, 37, 67, 68, or 69; and
b) a second Fc polypeptide comprising SEQ ID NO:29 or 56.

Certain embodiments provide the use of a protein in the preparation of a medicament for providing a clinical benefit to a subject with Hunter syndrome by administering a therapeutically effective dose of the medicament to the subject, wherein the medicament is capable of providing an improvement in a clinical outcome upon administration, as measured by one or more clinical Global Impression of Change instruments relative to baseline levels measured in the subject prior to administration, and wherein the protein comprises:
a) a fusion polypeptide comprising a first Fc polypeptide linked to an iduronate 2-sulfatase (IDS) amino acid sequence, wherein the fusion polypeptide comprises SEQ ID NO:35, 36, 37, 67, 68, or 69; and
b) a second Fc polypeptide comprising SEQ ID NO:29 or 56.

Certain embodiments provide a method of improving treatment in a patient having non-neuronopathic Hunter syndrome, comprising switching the patient from idursulfase enzyme replacement therapy to treatment with a pharmaceutical composition comprising a protein, wherein the protein comprises:
a) a fusion polypeptide comprising a first Fc polypeptide linked to an iduronate 2-sulfatase (IDS) amino acid sequence, wherein the fusion polypeptide comprises SEQ ID NO:35, 36, 37, 67, 68, or 69; and
b) a second Fc polypeptide comprising SEQ ID NO:29 or 56.

Certain embodiments provide a pharmaceutical composition comprising a protein for use in a method of improving treatment in a patient having non-neuronopathic Hunter syndrome, the method comprising switching the patient from idursulfase enzyme replacement therapy to treatment with the pharmaceutical composition, wherein the protein comprises:
a) a fusion polypeptide comprising a first Fc polypeptide linked to an IDS amino acid sequence, wherein the fusion polypeptide comprises SEQ ID NO:35, 36, 37, 67, 68, or 69; and
b) a second Fc polypeptide comprising SEQ ID NO:29 or 56.

Certain embodiments provide the use of a protein in the preparation of a medicament for improving treatment in a patient having non-neuronopathic Hunter syndrome by switching the patient from idursulfase enzyme replacement therapy to treatment with the medicament, wherein the protein comprises:
a) a fusion polypeptide comprising a first Fc polypeptide linked to an IDS amino acid sequence, wherein the fusion polypeptide comprises SEQ ID NO:35, 36, 37, 67, 68, or 69; and
b) a second Fc polypeptide comprising SEQ ID NO:29 or 56.

Certain embodiments provide a method of resolving an infusion-related reaction (IRR) in a subject receiving treatment for Hunter syndrome, wherein the subject is being intravenously administered or was intravenously administered an initial dose of a pharmaceutical composition comprising a protein, the method comprising reducing the amount and/or the infusion rate of administration of a subsequent dose of the pharmaceutical composition, wherein the protein comprises:
a) a fusion polypeptide comprising a first Fc polypeptide linked to an iduronate 2-sulfatase (IDS) amino acid sequence, wherein the fusion polypeptide comprises SEQ ID NO:35, 36, 37, 67, 68, or 69; and
b) a second Fc polypeptide comprising SEQ ID NO:29 or 56. In certain embodiments, the method comprises reducing the amount of the subsequent dose of the pharmaceutical composition. In certain embodiments, the method comprises reducing the infusion rate of administration of the subsequent dose of the pharmaceutical composition. In certain embodiments, the method comprises reducing the amount and the infusion rate of administration of the subsequent dose of the pharmaceutical composition.

Certain embodiments provide a pharmaceutical composition comprising a protein for use in a method of resolving an infusion-related reaction (IRR) in a subject receiving treatment for Hunter syndrome, wherein the subject is being intravenously administered or was intravenously administered an initial dose of the pharmaceutical composition, the method comprising reducing the amount and/or the infusion rate of administration of a subsequent dose of the pharmaceutical composition, wherein the protein comprises:
a) a fusion polypeptide comprising a first Fc polypeptide linked to an iduronate 2-sulfatase (IDS) amino acid sequence, wherein the fusion polypeptide comprises SEQ ID NO:35, 36, 37, 67, 68, or 69; and
b) a second Fc polypeptide comprising SEQ ID NO:29 or 56.

Certain embodiments also provide a method of resolving an IRR in a subject receiving treatment for Hunter syndrome, comprising modifying the subject's treatment regimen, wherein the treatment regimen comprises intravenous administration of an initial dose of a pharmaceutical composition comprising a protein, and the modified regimen comprises administering a subsequent dose of the pharmaceutical composition to the subject, wherein the subsequent dose is a reduced dose and/or is administered at a reduced rate of infusion, and wherein the protein comprises:
a) a fusion polypeptide comprising a first Fc polypeptide linked to an IDS amino acid sequence, wherein the fusion polypeptide comprises SEQ ID NO:35, 36, 37, 67, 68, or 69; and
b) a second Fc polypeptide comprising SEQ ID NO:29 or 56. In certain embodiments, the subsequent dose is a reduced dose. In certain embodiments, the subsequent dose is administered at a reduced rate of infusion. In certain embodiments, the subsequent dose is a reduced dose and is administered at a reduced rate of infusion.

Certain embodiments also provide a pharmaceutical composition comprising a protein for use in a method of resolving an IRR in a subject receiving treatment for Hunter syndrome, the method comprising modifying the subject's treatment regimen, wherein the treatment regimen comprises intravenous administration of an initial dose of a pharmaceutical composition comprising a protein, and the modified regimen comprises administering a subsequent dose of the pharmaceutical composition to the subject, wherein the subsequent dose is a reduced dose and/or is administered at a reduced rate of infusion, and wherein the protein comprises:
a) a fusion polypeptide comprising a first Fc polypeptide linked to an IDS amino acid sequence, wherein the fusion polypeptide comprises SEQ ID NO:35, 36, 37, 67, 68, or 69; and
b) a second Fc polypeptide comprising SEQ ID NO:29 or 56.

### BRIEF DESCRIPTION OF THE FIGURES

FIG. 1. Scheme showing dosing escalation and assessments for a Phase 1/2 study of patients diagnosed with MPS II.

FIG. 2. Cohort status and demographics of patients enrolled in Cohorts A and B of a Phase 1/2 study (interim analysis #1 safety population).

FIG. 3. Keratan sulfate (KS) levels in serum from patients in Cohort A (n=5). Normal range was measured using methods described herein in non-MPS pediatric controls (age range 0-9 years of age, median 5 years) from samples described in Bhalla et al. 2020. Int. J. Mol. Sci. 21:5188 (20 pages).

FIGS. 4A-4B. Heparan sulfate (HS) levels in serum (FIG. 4A) and urine (FIG. 4B) from patients in Cohort A (n=5). For serum HS, the normal range measured by LC-MS/MS is not yet determined. For urine HS, the preliminary normal range (10^{th} and 90^{th} percentile gray dashed bars) of urine HS was measured by a validated LC-MS/MS assay based on analysis of urine from 24 healthy children (age range 0-10 years, median 5 years).

FIG. 5. Dermatan sulfate (DS) levels in serum from patients in Cohort A (n=5). Normal range measured by LC-MS/MS is not yet determined.

FIG. 6. Total GAG levels in urine from patients in Cohort A (n=5) and Cohort B (n=12). Total urine GAG levels were measured by colorimetric assay. Upper limit of normal (ULN) of total urine GAGs/mmol creatinine is age dependent, ranging from 36 mg at ≤1 year to 5.5 mg at ≥14 years. Relevant ULN for each Cohort are represented by gray lines: 2-3 years ( [symbol] ); 6-7 years ( [symbol] ); and 10-11 years ( [symbol] ).

FIG. 7. Heparan sulfate (HS) levels in CSF from patients in Cohort A (n=5) and Cohort B (n=10). Preliminary normal range (10^{th} and 90^{th} percentile gray dashed bars) determined using 30 healthy adult CSF samples (age range 18-81 years, median 52 years). Total CSF GAG levels are similar in adults and children (Hendriksz et al., Mol. Genet. Metab. Rep., 5:103-106 (2015)). *One patient in Cohort B1 escalated to higher dose one week before Week 13 CSF collection. Timepoints on x-axis represent intended collection times and may vary by ˜1 week in some subjects.

FIG. 8. Dermatan sulfate (DS) levels in CSF from patients in Cohort A (n=5) and Cohort B (n=10). Preliminary normal range (10^{th} and 90^{th} percentile gray dashed bars) determined using 30 healthy adult CSF samples (age range 18-81 years, median 52 years). Total CSF GAG levels are similar in adults and children (Hendriksz et al., Mol. Genet. Metab. Rep., 5:103-106 (2015)). *One patient in Cohort B1 escalated to higher dose one week before Week 13 CSF collection. Timepoints on x-axis represent intended collection times and may vary by ˜1 week in some subjects.

FIG. 9. Ganglioside (GM3) levels in CSF from patients in Cohort A (n=5) and Cohort B (n=10). Preliminary normal range (10^{th} and 90^{th} percentile gray dashed bars) determined using 17 healthy adult CSF samples (age range 22-50 years, median 27 years). *One patient in Cohort B1 escalated to higher dose one week before Week 13. Timepoints at x-axis represent intended collection times and may vary by ˜1 week in some patients.

FIG. 10. BMP and Glucosylceramide levels in CSF from patients in Cohort A (n=5). Preliminary normal range (10^{th}, 50^{th}, 90^{th} percentile gray dashed lines) determined using 17 healthy adult CSF samples (age range 22-50 years, median 27 years).

FIGS. 11A-11B. Evaluation of subjects in Cohort A through week 24 (n=5) using Clinician Global Impression of Change (CGI-C) (FIG. 11A) and Parent/Caregiver Global Impression of Change (PGI-C) (FIG. 11B).

FIG. 12. KS levels in aged IDS KO; TfR^{mu/hu} KI mice and in mice cohorts treated with vehicle or ETV:IDS.

FIG. 13. Cohort status and demographics of patients enrolled in Cohorts A and B of a Phase 1/2 study (interim analysis #2 safety population).

FIG. 14. Heparan sulfate (HS) levels in urine (ug/mg) from patients in Cohorts A, B1, B2, and B3. The dashed lines represent the lower and upper bounds for the 10^{th} and 90^{th} percentiles from the normal range, which were determined from 24 healthy pediatric subjects (age range 0-10 years, median 5 years) using the same assay as the treated subjects.

FIG. 15. Dermatan sulfate (DS) levels in urine (ug/mg) from patients in Cohorts A, B1, B2, and B3. The dashed lines represent the lower and upper bounds for the 10^{th} and 90^{th} percentiles from the normal range, which were determined from 24 healthy pediatric subjects (age range 0-10 years, median 5 years) using the same assay as the treated subjects.

FIG. 16. Total GAG levels in urine from patients (n=20) in Cohorts A, B1, B2 and B3. Total urine GAG levels were measured by colorimetric assay. Upper limit of normal (ULN) of total urine GAGs/mmol creatinine is age dependent and range from 36 mg at ≤1 year to 5.5 mg at ≥14 years. Relevant ULN for each Cohort are represented by gray lines: 2-3 years ( [symbol] )<15.0 mg gags/mmol creatinine; 6-7 years ( [symbol] )<10.3 mg gags/mmol creatinine; and 10-11 years ( [symbol] )<8.2 mg gags/mmol creatinine.

FIG. 17. Heparan sulfate (HS) levels in CSF from patients in Cohorts A, B1, B2, and B3. CSF HS is based on the sum of 4 major disaccharides from HS (D0A0, D0S0, DOA6, and D2S6) following enzymatic digestion. The arrow indicates the trajectory of the non-neuronopathic MPS II (nnMPSII) patient in Cohort B2. The black dashed lines represent the lower and upper bounds for the 10^{th} (++) and 90^{th} (+) percentiles from the normal range, which were determined from 30 healthy adult CSF samples (age range 18-81 years, median 52 years) using the same assay as the treated subjects. Total CSF GAG levels are similar in adults and children (Hendriksz et al., Mol. Genet. Metab. Rep., 5:103-106 (2015)). The gray dashed lines represent the estimated range of attenuated (non-neuronopathic) CSF HS levels. Estimate uses proportionality of mean attenuated patient levels compared to mean severe/neuronopathic patient baseline levels in the study based on the 39% and 21% lower levels in attenuated vs. severe patients as reported by Nevoret, “RGX-121 gene therapy for severe mucopolysaccharidosis type II (MPS II): Interim results of an ongoing first in human trial”, WORLD 2021 (*) and Okuyama 2021. MoL Ther. 29(2):671-679 (**), respectively.

FIG. 18. Dermatan sulfate (DS) levels in CSF from patients in Cohorts A, B1, B2, and B3. The black dashed lines represent the lower and upper bounds for the 10^{th} and 90^{th} percentiles from the normal range, which were determined from 30 healthy adult CSF samples (age range 18-81 years, median 52 years) using the same assay as the treated subjects. Total CSF GAG levels are similar in adults and children (Hendriksz et al., Mol. Genet. Metab. Rep., 5:103-106 (2015)).

FIG. 19. Ganglioside (GM3(d36:1))) levels in CSF from patients Cohorts A, B1, B2, and B3. The black dashed lines represent the lower and upper bounds for the 10^{th} and 90^{th} percentiles from the normal range, which were determined from 30 healthy adult CSF samples (age range 18-81 years, median 52 years) using the same assay as the treated subjects.

FIGS. 20A-20B. Evaluation of subjects in clinical outcomes population (N=17) from Cohort A and Cohort B at Week 24 of the study, using Clinician Global Impression of Change (CGI-C) (FIG. 20A) and Parent/Caregiver Global Impression of Change (PGI-C) (FIG. 20B).

FIG. 21. Updated cohort status and demographics of patients enrolled in Cohorts A, B, C and D of a Phase 1/2 study (safety population).

FIG. 22. Proportion of infusions associated with IRRs in the safety population.

FIGS. 23A-23B. Heparan sulfate (HS) levels (FIG. 23A) and Dermatan sulfate (DS) levels (FIG. 23B) in urine (ug/mg) from patients in Cohorts A, B1, B2, and B3. The dashed lines represent the lower and upper bounds for the 10^{th} and 90^{th} percentiles from the normal range, which were determined from 24 healthy pediatric subjects (age range 0-10 years, median 5 years) using the same assay as the treated subjects. Normal range urine HS (ug/mg): 1.61-3.6; normal range urine DS (ug/mg): 0.34-0.83.

FIG. 24. Heparan sulfate (HS) levels in CSF from patients in Cohorts A, B1, B2, and B3. CSF HS is based on the sum of 4 major disaccharides from HS (D0A0, D0S0, DOA6, and D2S6) following enzymatic digestion. The dashed lines represent the lower and upper bounds for the 10^{th} and 90^{th} percentiles from the normal range, which were determined from 30 healthy adult CSF samples (age range 18-81 years, median 52 years) using the same assay as the treated subjects. Total CSF GAG levels are similar in adults and children (Hendriksz et al., Mol. Genet. Metab. Rep., 5:103-106 (2015)). Normal range for CSF HS (ng/mL): 39.1-92.51.

FIGS. 25A-25B. Ganglioside levels (GM2(d36:1) FIG. 25A; GM3(d36:1) FIG. 25B) in CSF from patients Cohorts A, B1, B2, and B3. The dashed lines represent the lower and upper bounds for the 10^{th} and 90^{th} percentiles from the normal range, which were determined from 30 healthy adult CSF samples (age range 18-81 years, median 52 years) using the same assay as the treated subjects. Normal range for CSF GM2(d36:1) (ng/mL): 2.72-8.2; normal range for CSF GM3(d36:1) (ng/mL): 1.99-3.55.

FIGS. 26A-26B. Evaluation of subjects in clinical outcomes population (N=12) from Cohort A and Cohort B at Week 49 of the study, using Clinician Global Impression Scales of Change (CGI-C) (FIG. 26A) and Caregiver Global Impression Scales of Change (CaGI-C) (FIG. 26B).

### DETAILED DESCRIPTION

Certain methods of providing added peripheral IDS activity relative to a standard-of-care treatment to a subject with Hunter syndrome are provided herein. Such methods may comprise administering to the subject a therapeutically effective dose of a pharmaceutical composition comprising a protein, wherein the protein comprises an IDS amino acid sequence and a transferrin receptor (TfR) binding domain. Examples of proteins that comprise an IDS amino acid sequence and a TfR binding domain are described below, including ETV:IDS. As described herein, the term “ETV:IDS” refers to a protein comprising a first Fc polypeptide that is linked to an IDS enzyme, IDS enzyme variant, or a catalytically active fragment thereof, and a second Fc polypeptide; wherein the first and/or second Fc polypeptide is a modified Fc that is capable of binding (e.g., specifically binding) to a TfR (i.e., the Fc comprises a TfR binding domain described herein). ETV:IDS is being investigated as a potential treatment for patients with MPS II to address both the peripheral and central nervous system (CNS) symptoms of disease. Described herein are methods of treatment using ETV:IDS to provide a clinical benefit to a subject with Hunter syndrome, as well as to treat a patient having non-neuronopathic Hunter syndrome. Also provided herein are methods of resolving an IRR in a subject receiving treatment for Hunter syndrome, wherein the treatment comprises administration of ETV:IDS.

### Added Peripheral IDS Activity

Keratan sulfate (KS) is a glycosaminoglycan (GAG) species that is present in articular and growth plate cartilage where bone growth occurs. It is catabolized by lysosomal enzymes such as N-acetylgalactosamine-6-sulfate sulfatase and beta-galactosidase, deficiencies of which cause mucopolysaccharidosis IVA (MPS IVA) and mucopolysaccharidosis IVB (MPS IVB), respectively. KS has also been shown to be elevated in the urine and serum of Hunter syndrome (MPS II) patients (Tomatsu et al. 2005. J. Inheri. Metab. Dis. 28:187-202). However, it has not previously been shown that KS is responsive or can be used as a pharmacodynamic biomarker to evaluate compositions or therapies for treatment of Hunter Syndrome. For example, KS levels remained unchanged in Hunter syndrome (MPS II) patients treated with standard-of-care treatment (idursulfase) or hematopoietic stem cell therapy (HSCT) (Fujitsuka et al. 2019. Mol Genet Metab Rep. 19:100455). As described herein, treatment with a protein comprising an IDS amino acid sequence and a TfR binding domain (e.g., ETV:IDS), or a pharmaceutical composition comprising the same, may be used to reduce and/or normalize serum KS levels.

Accordingly, described herein are methods of using a protein comprising an IDS amino acid sequence and a TfR binding domain (e.g., ETV:IDS) to provide added peripheral IDS activity relative to a standard-of-care treatment to a subject with Hunter syndrome (e.g., a subject in need thereof), wherein the added peripheral activity is indicated by a reduction and/or normalization of serum keratan sulfate (KS) levels in the subject. In some embodiments, the added peripheral activity is also indicated by a reduction and/or normalization (e.g., sustained normalization) of urine glycosaminoglycans (GAGs) in the subject.

Thus, certain embodiments provide a method of reducing and/or normalizing serum KS levels in a subject with Hunter syndrome (e.g., a subject in need thereof), comprising administering to the subject a therapeutically effective dose of a pharmaceutical composition comprising a protein, wherein the administration reduces and/or normalizes KS levels in the subject relative to a control or baseline level, and wherein the protein comprises an IDS amino acid sequence and a transferrin receptor (TfR) binding domain (e.g., ETV:IDS). In certain embodiments, the control or baseline level is measured in the subject prior to the administration of the pharmaceutical composition. In certain embodiments, the control or baseline level is measured in a healthy subject or in a subject that does not have Hunter syndrome.

Certain embodiments also relate to a method of providing added peripheral IDS activity relative to a standard-of-care treatment to a subject with Hunter syndrome (e.g., a subject in need thereof), comprising administering to the subject a therapeutically effective dose of a pharmaceutical composition comprising a protein, wherein the protein comprises an IDS amino acid sequence and a transferrin receptor (TfR) binding domain (e.g., ETV:IDS), wherein the administration of the pharmaceutical composition reduces serum levels of keratan sulfate (KS) in the subject relative to a control or baseline level, and wherein the reduced serum levels of KS correlate with added peripheral IDS activity for the subject. In certain embodiments, the control or baseline level is measured in the subject prior to the administration of the pharmaceutical composition. In certain embodiments, the control or baseline level is measured in a healthy subject or in a subject that does not have Hunter syndrome.

In some embodiments, administration of the pharmaceutical composition reduces serum levels of KS relative to a baseline level (e.g., relative to a baseline level measured in the subject prior to administration). In certain embodiments, KS levels are reduced by at least about 20%, 25%, 30%, or 35% relative to a baseline level.

In some embodiments, administration of the pharmaceutical composition reduces serum levels of keratan sulfate (KS) in the subject to a baseline level measured in a healthy subject or in a subject that does not have Hunter syndrome. Thus, in certain embodiments, administration of the pharmaceutical composition normalizes serum levels of KS in the subject. In certain embodiments, the normalization is sustained normalization.

Certain embodiments provide a method for evaluating peripheral IDS activity in a subject having Hunter syndrome, comprising measuring the level of KS in a sample (e.g., serum) from the subject obtained at a time point after administration of a pharmaceutical composition comprising a protein; wherein a decrease in KS levels in the sample obtained after administration of the pharmaceutical composition as compared to the levels of KS in a sample (e.g., serum) from the subject obtained prior to administration of the pharmaceutical composition indicates added peripheral IDS activity in the subject, and wherein the protein comprises an IDS amino acid sequence and a transferrin receptor (TfR) binding domain. In certain embodiments, a decrease in KS levels is detected. In certain embodiments, such a subject is identified as a candidate for treatment. In certain embodiments, such a subject is identified as a candidate for adjusting a treatment regimen for the subject.

Certain embodiments also provide a method for evaluating peripheral IDS activity in a subject having Hunter syndrome (e.g., in a subject in need thereof), comprising:
(a) determining a first marker profile by detecting the level of keratan sulfate (KS) in a sample (e.g., serum) from the subject obtained at baseline or prior to initiation of treatment with a pharmaceutical composition comprising a protein;
(b) administering to the subject the pharmaceutical composition comprising the protein;
(c) determining a second marker profile by detecting the level of KS in a sample (e.g., serum) from the subject obtained at a time point after administration of the pharmaceutical composition comprising the protein; and
(d) comparing the first and the second marker profiles;
wherein a decrease in KS levels between the first and the second marker profiles indicates added peripheral IDS activity for the subject; and wherein the protein comprises an IDS amino acid sequence and a transferrin receptor (TfR) binding domain (e.g., ETV:IDS). In certain embodiments, a decrease in KS levels is detected. In certain embodiments, such a method further comprises the additional administration of the pharmaceutical composition (e.g., as part of a treatment regimen).

Certain embodiments provide a method of providing added peripheral iduronate 2-sulfatase (IDS) activity relative to a standard-of-care treatment to a subject with Hunter syndrome (e.g., in a subject in need thereof), comprising administering to the subject a therapeutically effective dose of a pharmaceutical composition comprising a protein (e.g., administering at least 23 weekly therapeutically effective doses), wherein the administration of the pharmaceutical composition normalizes levels of urine heparan sulfate (HS) and/or dermatan sulfate (DS) in the subject by at least 12 weekly doses, and wherein normalization is sustained after at least 23 weekly doses, and wherein the protein comprises an IDS amino acid sequence and a transferrin receptor (TfR) binding domain.

Certain embodiments also provide a method of normalizing urine heparan sulfate (HS) and/or dermatan sulfate (DS) levels in a subject with Hunter syndrome (e.g., in a subject in need thereof), comprising administering to the subject a therapeutically effective dose of a pharmaceutical composition comprising a protein (e.g., administering at least 23 weekly therapeutically effective doses), wherein the HS and/or DS levels in the subject are normalized by at least 12 weekly doses, wherein normalization is sustained after at least 23 weekly doses, and wherein the protein comprises an IDS amino acid sequence and a transferrin receptor (TfR) binding domain.

Certain embodiments provide a method for evaluating peripheral IDS activity in a subject having Hunter syndrome, comprising measuring the levels of HS and/or DS in a urine sample from the subject obtained at a time point after weekly administration of a pharmaceutical composition comprising a protein (e.g., after 23 weekly doses); wherein a decrease in HS and/or DS levels in the sample obtained after administration of the pharmaceutical composition to a baseline level measured in a healthy subject or in a subject that does not have Hunter syndrome indicates added peripheral IDS activity in the subject, and wherein the protein comprises an IDS amino acid sequence and a transferrin receptor (TfR) binding domain. In certain embodiments, a decrease in HS and/or DS levels is detected. In certain embodiments, such a subject is identified as a candidate for treatment. In certain embodiments, such a subject is identified as a candidate for adjusting a treatment regimen for the subject.

Certain embodiments provide a method for evaluating peripheral IDS activity in a subject having Hunter syndrome (e.g., in a subject in need thereof), comprising:
(a) determining a first marker profile by detecting the levels of heparan sulfate (HS) and/or dermatan sulfate (DS) in a urine sample from the subject obtained at baseline or prior to initiation of treatment with a pharmaceutical composition comprising a protein;
(b) administering to the subject the pharmaceutical composition comprising the protein (e.g., administering the pharmaceutical composition weekly for at least 23 weeks);
(c) determining a second marker profile by detecting the levels HS and/or DS in a urine sample from the subject obtained at a time point after administration of the pharmaceutical composition comprising the protein (e.g., after weekly doses); and
(d) comparing the first and the second marker profiles to a baseline level measured in a healthy subject or in a subject that does not have Hunter syndrome;
wherein a decrease in HS and/or DS levels in the second marker profile to a baseline level measured in a healthy subject or in a subject that does not have Hunter syndrome indicates added peripheral IDS activity in the subject, and
wherein the protein comprises an IDS amino acid sequence and a transferrin receptor (TfR) binding domain. In certain embodiments, a decrease in HS and/or DS levels is detected.

In certain embodiments, the subject was previously treated with idursulfase enzyme replacement therapy (also referred to as “standard-of-care” therapy). In certain embodiments, the subject was switched from idursulfase enzyme replacement therapy to treatment with the pharmaceutical composition without a washout period. Accordingly, in certain embodiments, the reduction and/or normalization of serum KS levels relative to a baseline level measured in the subject prior to administration of the pharmaceutical composition is indicative of added peripheral IDS activity.

As described herein, the pharmaceutical composition may be administered weekly. Thus, in certain embodiments, a method described herein comprises administering the pharmaceutical composition weekly for a period of time described herein, such as, e.g., at least about 12 weeks, at least about 23 weeks, at least about 48 weeks, or more.

In certain embodiments, the reduction and/or normalization of serum KS levels occurs by about 5, 9, 13, 24 or more weeks after the initial administration of the pharmaceutical composition. In certain embodiments, the reduction and/or normalization of KS is sustained after at least 12 weekly doses. In certain embodiments, the reduction and/or normalization of KS is sustained after at least 23 weekly doses.

In certain embodiments, the subject's serum heparan sulfate (HS) and/or dermatan sulfate (DS) levels decrease after administration of the pharmaceutical composition. In certain embodiments, the subject's serum heparan sulfate (HS) and/or dermatan sulfate (DS) levels decrease by about 50%, 55%, 60%, or 65% relative to a baseline level after administration of the pharmaceutical composition. In certain embodiments, the subject's serum heparan sulfate (HS) and/or dermatan sulfate (DS) levels normalize after administration of the pharmaceutical composition.

In certain embodiments, the subject's serum HS and/or DS levels decrease and/or normalize relative to a baseline level by at least 12 weekly doses of the pharmaceutical composition. In certain embodiments, the subject's serum HS and/or DS levels decrease and/or normalize relative to a baseline level by at least 23 weekly doses of the pharmaceutical composition. In certain embodiments, the subject's serum HS and/or DS levels decrease and/or normalize relative to a baseline level by at least 48 weekly doses of the pharmaceutical composition.

In certain embodiments, the subject's urine heparan sulfate (HS) and/or dermatan sulfate (DS) levels decrease after administration of the pharmaceutical composition. In certain embodiments, the subject's urine HS and/or DS levels decrease and/or normalize relative to a baseline level by at least 12 weekly doses of the pharmaceutical composition. In certain embodiments, the subject's urine HS and/or DS levels decrease and/or normalize relative to a baseline level by at least 23 weekly doses of the pharmaceutical composition. In certain embodiments, the subject's urine HS and/or DS levels decrease and/or normalize relative to a baseline level by at least 48 weekly doses of the pharmaceutical composition.

In certain embodiments, the normalization of urine HS and/or DS is sustained after at least 12 weekly doses. In certain embodiments, the normalization of urine HS and/or DS is sustained after at least 23 weekly doses. In certain embodiments, the normalization of urine HS and/or DS is sustained after at least 30 weekly doses. In certain embodiments, the normalization of urine HS and/or DS is sustained after at least 36 weekly doses. In certain embodiments, the normalization of urine HS and/or DS is sustained after at least 42 weekly doses. In certain embodiments, the normalization of urine HS and/or DS is sustained after at least 48 weekly doses.

In certain embodiments, the reduction and/or normalization of serum and/or urine HS and/or DS levels relative to a baseline level after administration of the pharmaceutical composition is indicative of added peripheral IDS activity.

In certain embodiments, the subject's total urine GAG levels decrease relative to a baseline level after administration of the pharmaceutical composition. In certain embodiments, the subject's total urine GAG levels decrease relative to a baseline level after at least 12 weekly doses of the pharmaceutical composition. In certain embodiments, the subject's total urine GAG levels decrease relative to a baseline level after at least 23 weekly doses of the pharmaceutical composition. In certain embodiments, the subject's total urine GAG levels decrease relative to a baseline level after at least 48 weekly doses of the pharmaceutical composition. In the foregoing embodiments, the baseline level is a baseline level measured in the subject prior to the administration of the pharmaceutical composition.

In certain embodiments, the subject's total urine GAG levels decrease relative to a baseline level by at least the 12^{th} weekly dose. In certain embodiments, the subject's total urine GAG levels decrease relative to a baseline level by at least the 23^{rd} weekly dose. In certain embodiments, the subject's total urine GAG levels decrease relative to a baseline level by at least the 48^{th} weekly dose. In the foregoing embodiments, the baseline level is a baseline level measured in the subject prior to the administration of the pharmaceutical composition.

In certain embodiments, a method described herein comprises measuring at least one analyte of interest (e.g., KS, HS and/or DS). In certain embodiments, an analyte of interest (e.g., KS, HS or DS levels) are measured using a method known in the art, such as a method described herein. In certain embodiments, serum KS levels are measured using a method known in the art, such as a method described herein.

### Clinical Benefit

Described herein are methods of treatment using ETV:IDS to provide a clinical benefit to a subject with Hunter syndrome. As discussed herein, ETV:IDS is a transferrin receptor (TfR) binding fusion protein that comprises an IDS amino acid sequence and is capable of crossing the blood brain barrier (BBB) and treating the peripheral and CNS manifestations of Hunter syndrome. For example, as described herein, subjects receiving ETV:IDS therapy were shown to have improved clinical outcomes as measured by clinical Global Impression of Change instruments (see, Example 1).

Accordingly, certain embodiments provide a method of providing a clinical benefit to a subject with Hunter syndrome (e.g., a subject in need thereof), comprising administering to the subject a therapeutically effective dose of a pharmaceutical composition comprising ETV:IDS protein, wherein the administration of the pharmaceutical composition produces an improvement in a clinical outcome as measured by one or more clinical Global Impression of Change instruments (e.g., CGI-C and/or PGI-C or CaGI-C) relative to baseline levels in the subject measured prior to the administration.

Clinical Global Impression of Change instruments (e.g., CGI-C, PGI-C, or CaGI-C, described below) are evaluations that may be performed by a clinician or a parent/caregiver to give a stand-alone assessment of a patient's global functioning prior to and after initiating a treatment (see, Guy W, editor. ECDEU Assessment Manual for Psychopharmacology. Rockville, MD: US Department of Health, Education, and Welfare Public Health Service Alcohol, Drug Abuse, and Mental Health Administration; 1976, which is incorporated by reference herein for all purposes). The instruments provide an overall measure that takes into account all available information, including, e.g., a knowledge of the patient's history, psychosocial circumstances, symptoms, behavior, and the impact of the symptoms on the patient's ability to function. The instruments compare a patient's condition to a period prior to the initiation of treatment (i.e., a subject's pre-treatment baseline level), such as the one-week period prior to initiation of treatment. The change in the subject's condition is then graded. The subject's overall disease manifestations or an aspect of the disease (e.g., cognitive, behavioral, or physical) may be evaluated using these assessments. Thus, a “clinical benefit” or an “improved clinical outcome”, as evaluated by the instruments may refer to an improvement in a subject's overall Hunter syndrome symptoms or to a particular aspect of the disease. For example, approximately two-thirds of Hunter syndrome patients display the neuronopathic form of the disease, which in addition to earlier presentation of the somatic disease, is characterized by progressive, debilitating neurobehavioral deficits, which include but are not limited to, motor skill deficits, cognitive deficits (e.g., learning and memory deficits) and sensorimotor gating deficits (e.g., attention and inhibitory function deficits). Thus, the Global Impression of Change instruments may be used to evaluate cognitive abilities, behavior or physical abilities of a subject with Hunter syndrome.

In certain embodiments, a method described herein comprises evaluating the subject's condition (e.g., the subject's overall disease manifestations or an aspect of the disease (e.g., cognitive, behavioral, or physical abilities)) using clinical Global Impressions of Change instruments (e.g., CGI-C and/or PGI-C or CaGI-C) prior to initiation of treatment with the pharmaceutical composition to obtain baseline levels in the subject. In certain embodiments, a method described herein comprises evaluating the subject's condition (e.g., the subject's overall disease manifestations or an aspect of the disease (e.g., cognitive, behavioral, or physical abilities)) using the clinical Global Impressions of Change instruments after administration of the pharmaceutical composition. In certain embodiments, an improved clinical outcome is determined by comparing the subject's condition after administration of the pharmaceutical composition with the obtained baseline levels of the subject.

In certain embodiments, the clinical outcome is measured by the Clinician Global Impression of Change instrument (CGI-C). In certain embodiments, the clinical outcome is measured by the Parent/Caregiver Global Impression of Change instrument (PGI-C). In certain embodiments, the clinical outcome is measured by the Caregiver Global Impression of Change (CaGI-C). In certain embodiments, the clinical outcome is measured by CGI-C and either PGI-C or CaGI-C.

In certain embodiments, CGI-C shows improvement of a measured clinical outcome (e.g., at about 5, 9, 13, 24, 49 or more weeks after the initial administration of the pharmaceutical composition). In certain embodiments, PGI-C or CaGI-C shows improvement of a measured clinical outcome (e.g., at about 5, 9, 13, 24, 49 or more weeks after the initial administration of the pharmaceutical composition). In certain embodiments, the combination of CGI-C and PGI-C or CaGI-C show improvement of a measured clinical outcome (e.g., at about 5, 9, 13, 24, 49 or more weeks after the initial administration of the pharmaceutical composition).

As described herein, the pharmaceutical composition may be administered weekly. Thus, in certain embodiments, a method described herein comprises administering the pharmaceutical composition weekly for a period of time described herein, such as, e.g., at least about 12 weeks, at least about 23 weeks, at least about 48 weeks, or more.

Thus, in certain embodiments, CGI-C shows improvement of a measured clinical outcome by at least 12 weekly doses of the pharmaceutical composition. In certain embodiments, CGI-C shows improvement of a measured clinical outcome by at least 23 weekly doses of the pharmaceutical composition. In certain embodiments, CGI-C shows improvement of a measured clinical outcome by at least 48 weekly doses of the pharmaceutical composition. In certain embodiments, PGI-C or CaGI-C shows improvement of a measured clinical outcome by at least 12 weekly doses of the pharmaceutical composition. In certain embodiments, PGI-C or CaGI-C shows improvement of a measured clinical outcome by at least 23 weekly doses of the pharmaceutical composition. In certain embodiments, PGI-C or CaGI-C shows improvement of a measured clinical outcome by at least 48 weekly doses of the pharmaceutical composition. In certain embodiments, the combination of CGI-C and PGI-C or CaGI-C show improvement of a measured clinical outcome by at least 12 weekly doses of the pharmaceutical composition. In certain embodiments, the combination of CGI-C and PGI-C or CaGI-C show improvement of a measured clinical outcome by at least 23 weekly doses of the pharmaceutical composition. In certain embodiments, the combination of CGI-C and PGI-C or CaGI-C show improvement of a measured clinical outcome by at least 48 weekly doses of the pharmaceutical composition.

In certain embodiments, the measured clinical outcome based on CGI-C and/or PGI-C is minimally improved. In certain embodiments, the measured clinical outcome based on CGI-C and/or PGI-C is much improved. In certain embodiments, the measured clinical outcome based on CGI-C and/or PGI-C is very much improved. In certain embodiments, the improved clinical outcome based on CGI-C and/or PGI-C is the subject's overall symptoms. In certain embodiments, the improved clinical outcome based on CGI-C and/or PGI-C is the subject's cognitive abilities and/or behavior. In certain embodiments, the improved clinical outcome based on CGI-C and/or PGI-C is the subject's cognitive abilities. In certain embodiments, the improved clinical outcome based on CGI-C and/or PGI-C is the subject's behavior. In certain embodiments, the improved clinical outcome based on CGI-C and/or PGI-C is the subject's physical abilities.

In certain embodiments, the measured clinical outcome based on CGI-C and/or CaGI-C is a little improved. In certain embodiments, the measured clinical outcome based on CGI-C and/or CaGI-C is much improved. In certain embodiments, the improved clinical outcome based on CGI-C and/or CaGI-C is the subject's overall MIPS II symptoms. In certain embodiments, the improved clinical outcome based on CGI-C and/or CaGI-C is the subject's communication, daily living skills, problematic behavior and/or social skills. In certain embodiments, the improved clinical outcome based on CGI-C and/or CaGI-C is the subject's social skills. In certain embodiments, the improved clinical outcome based on CGI-C and/or CaGI-C is the subject's problematic behavior. In certain embodiments, the improved clinical outcome based on CGI-C and/or CaGI-C is the subject's physical abilities. In certain embodiments, the improved clinical outcome based on CGI-C and/or CaGI-C is the subject's communication. In certain embodiments, the improved clinical outcome based on CGI-C and/or CaGI-C is the subject's daily living skills.

In some embodiments, administration of the pharmaceutical composition results in a reduction (e.g., a sustained reduction) of a glycosaminoglycan (GAG) in the CSF of the subject relative to a baseline level in the subject measured prior to the administration of the pharmaceutical composition. In some embodiments, the administration results in reducing the level of the CSF GAG in the subject to a level measured in a healthy subject or a subject that does not have Hunter syndrome. In certain embodiments, the GAG is heparan sulfate. In certain embodiments, the GAG is dermatan sulfate. In some embodiments, the administration results in a reduction in urine total GAG levels in the subject relative to a baseline level in the subject measured prior to the administration of the pharmaceutical composition. In some embodiments, the reduction in urine total GAG levels in the subject is indicative of added peripheral activity.

In certain embodiments, the level of a GAG in the CSF of the subject decreases to a level measured in a healthy subject or a subject that does not have Hunter syndrome by at least the 6th weekly dose, by at least the 12th weekly dose, by at least the 23rd weekly dose, or by at least the 48th weekly dose of the pharmaceutical composition.

In certain embodiments, the normalization of a GAG level in the CSF of the subject is sustained after at least 12 weekly doses. In certain embodiments, the normalization of a GAG level in the CSF of the subject is sustained after at least 23 weekly doses. In certain embodiments, the normalization of a GAG level in the CSF of the subject is sustained after at least 30 weekly doses. In certain embodiments, the normalization of a GAG level in the CSF of the subject is sustained after at least 36 weekly doses. In certain embodiments, the normalization of a GAG level in the CSF is sustained after at least 42 weekly doses. In certain embodiments, the normalization of a GAG level in the CSF of the subject is sustained after at least 48 weekly doses.

In certain embodiments, the level of a ganglioside in the CSF of the subject decreases to a level measured in a healthy subject or a subject that does not have Hunter syndrome by at least the 6th weekly dose, by at least the 12th weekly dose, by at least the 23rd weekly dose, or by at least the 48th weekly dose of the pharmaceutical composition. In some embodiments, the ganglioside is GM2. In some embodiments, the ganglioside is GM3.

In certain embodiments, the normalization of a ganglioside level in the CSF of the subject is sustained after at least 12 weekly doses. In certain embodiments, the normalization of a ganglioside level in the CSF of the subject is sustained after at least 23 weekly doses. In certain embodiments, the normalization of a ganglioside level in the CSF of the subject is sustained after at least 30 weekly doses. In certain embodiments, the normalization of a ganglioside level in the CSF of the subject is sustained after at least 36 weekly doses. In certain embodiments, the normalization of a ganglioside level in the CSF is sustained after at least 42 weekly doses. In certain embodiments, the normalization of a ganglioside level in the CSF of the subject is sustained after at least 48 weekly doses. In some embodiments, the ganglioside is GM2. In some embodiments, the ganglioside is GM3.

In certain embodiments, the subject's total urine GAG levels decrease relative to a baseline level after at least 12 weekly doses of the pharmaceutical composition. In certain embodiments, the subject's total urine GAG levels decrease relative to a baseline level after at least 23 weekly doses of the pharmaceutical composition. In certain embodiments, the subject's total urine GAG levels decrease relative to a baseline level after at least 48 weekly doses of the pharmaceutical composition. In the foregoing embodiments, the baseline level is a baseline level measured in the subject prior to the administration of the pharmaceutical composition.

In certain embodiments, the subject's total urine GAG levels decrease relative to a baseline level by at least the 12^{th} weekly dose. In certain embodiments, the subject's total urine GAG levels decrease relative to a baseline level by at least the 23^{rd} weekly dose. In certain embodiments, the subject's total urine GAG levels decrease relative to a baseline level by at least the 48^{th} weekly dose. In the foregoing embodiments, the baseline level is a baseline level measured in the subject prior to the administration of the pharmaceutical composition.

### Certain Methods for Treating Non-Neuronopathic Hunter Syndrome

Provided herein is a method of improving treatment in a patient having non-neuronopathic Hunter syndrome (e.g., in a subject in need thereof), wherein the method comprises switching the patient from idursulfase enzyme replacement therapy to treatment with a pharmaceutical composition comprising ETV:IDS. In certain embodiments, improved treatment may be determined by evaluating the level of a glycosaminoglycan (GAG) in the cerebrospinal fluid (CSF) of the patient and/or the total GAG levels in the urine of the patient. Thus, in some embodiments, the method results in a reduction (e.g., a sustained reduction) of a GAG in the CSF of the patient relative to a baseline level in the patient measured before the switching. In some embodiments, the method results in reducing the level of the CSF GAG in the patient to a level measured in a healthy subject or a subject that does not have Hunter syndrome. In certain embodiments, the GAG is heparan sulfate. In certain embodiments, the GAG is dermatan sulfate. In some embodiments, the method results in a reduction in urine total GAG levels in the patient relative to a baseline level in the patient measured before the switching. In some embodiments, the reduction in urine total GAG levels in the patient is indicative of added peripheral activity.

As described herein, treatment with the pharmaceutical composition may comprise administering the pharmaceutical composition weekly. Thus, in certain embodiments, a method described herein comprises administering the pharmaceutical composition weekly for a period of time described herein, such as, e.g., at least about 12 weeks, at least about 23 weeks, at least about 48 weeks, or more.

In certain embodiments, the level of a GAG in the CSF of the patient decreases to a level measured in a healthy subject or a subject that does not have Hunter syndrome by at least the 6th weekly dose, by at least the 12th weekly dose, by at least the 23rd weekly dose, or by at least the 48th weekly dose of the pharmaceutical composition.

In certain embodiments, the normalization of a GAG level in the CSF of the patient is sustained after at least 12 weekly doses. In certain embodiments, the normalization of a GAG level in the CSF of the patient is sustained after at least 23 weekly doses. In certain embodiments, the normalization of a GAG level in the CSF of the patient is sustained after at least 30 weekly doses. In certain embodiments, the normalization of a GAG level in the CSF of the patient is sustained after at least 36 weekly doses. In certain embodiments, the normalization of a GAG level in the CSF is sustained after at least 42 weekly doses. In certain embodiments, the normalization of a GAG level in the CSF of the patient is sustained after at least 48 weekly doses.

In certain embodiments, the patient's total urine GAG levels decrease relative to a baseline level after at least 12 weekly doses of the pharmaceutical composition. In certain embodiments, the patient's total urine GAG levels decrease relative to a baseline level after at least 23 weekly doses of the pharmaceutical composition. In certain embodiments, the patient's total urine GAG levels decrease relative to a baseline level after at least 48 weekly doses of the pharmaceutical composition. In the foregoing embodiments, the baseline level is a baseline level measured in the patient before the switching.

In certain embodiments, the patient's total urine GAG levels decrease relative to a baseline level by at least the 12^{th} weekly dose. In certain embodiments, the patient's total urine GAG levels decrease relative to a baseline level by at least the 23^{rd} weekly dose. In certain embodiments, the patient's total urine GAG levels decrease relative to a baseline level by at least the 48^{th} weekly dose. In the foregoing embodiments, the baseline level is a baseline level measured in the patient before the switching.

### Resolving Infusion Related Reactions

Described herein are methods and treatment regimens for resolving infusion related reactions (IRRs) associated with the intravenous administration of ETV:IDS. Certain subjects receiving intravenous ETV:IDS have experienced IRRs associated with the administration. These IRRs were successfully resolved using reduced dosing and/or reduced infusion rates, optionally in combination with the administration of pre-treatment medications that are useful for treating IRRs (see, e.g., Example 1).

Accordingly, certain embodiments provide a method of resolving an infusion-related reaction (IRR) in a subject receiving treatment for Hunter syndrome, wherein the subject is being intravenously administered or was intravenously administered an initial dose of a pharmaceutical composition comprising ETV:IDS protein, the method comprising reducing the amount and/or reducing the infusion rate of administration of a subsequent dose of the pharmaceutical composition. The reduced dose and rate are relative to the dose and rate of the initial dose. In certain embodiments, the method comprises reducing the amount of the subsequent dose of the pharmaceutical composition. In certain embodiments, the method comprises reducing the infusion rate of administration of the subsequent dose of the pharmaceutical composition. In certain embodiments, the method comprises reducing the amount and reducing the infusion rate of administration of the subsequent dose of the pharmaceutical composition.

Certain embodiments also provide a method of resolving an IRR in a subject receiving treatment for Hunter syndrome, comprising modifying the subject's treatment regimen, wherein the treatment regimen comprises intravenous administration of an initial dose of a pharmaceutical composition comprising ETV:IDS protein, and the modified regimen comprises administering a subsequent dose of the pharmaceutical composition to the subject, wherein the subsequent dose is a reduced dose and/or is administered at a reduced rate of infusion. The reduced dose and rate are relative to the dose and rate in the initial regimen. In certain embodiments, the subsequent dose is a reduced dose. In certain embodiments, the subsequent dose is administered at a reduced rate of infusion. In certain embodiments, the subsequent dose is a reduced dose and is administered at a reduced rate of infusion.

As used herein, an “infusion related reaction (IRR)” refers to an adverse reaction to the infusion of a pharmacological or biological substance, which typically develops during or shortly after administration. Mild to moderate infusion reactions are associated with, e.g., chills, fever, mild hypotension, dyspnea and/or rash. Severe reactions are less common and are, amongst other symptoms, associated with, e.g., severe hypotension, anaphylaxis (including, e.g., anaphylaxis based on the Sampson criteria) and/or cardiac dysfunction.

In certain embodiments, the IRR is a mild or moderate infusion reaction. In certain embodiments, the subject develops a fever.

In certain embodiments, the IRR is a severe infusion reaction. In certain embodiments, the IRR is anaphylaxis. In certain embodiments, the anaphylaxis meets the Sampson criteria (Sampson, et al., J Allergy Clin Immunol. 2006; 117:391-397).

### Proteins Comprising IDS Enzyme(s) and a TfR Binding Domain, Including ETV:IDS Embodiments

Described below are certain embodiments of proteins that comprise an IDS amino acid sequence (i.e., an IDS enzyme, IDS enzyme variant, or a catalytically active fragment thereof (e.g., a wild-type IDS (e.g., a wild-type human IDS), wild-type IDS variant, or a catalytically active fragment thereof) and a transferrin receptor (TfR) binding domain. As used herein, the term “transferrin receptor binding domain” or “TfR binding domain” refers to a peptide or protein, or fragment thereof, that is capable of specifically binding to a transferrin receptor protein. Such TfR binding domains may be used to facilitate delivery of a peptide or protein (e.g., a protein comprising an IDS enzyme, IDS enzyme variant, or a catalytically active fragment thereof), across the blood-brain barrier (BBB).

In certain embodiments, the protein comprises an IDS amino acid sequence linked to an Fc polypeptide; and a TfR binding domain. In some cases, the protein includes a dimeric Fc polypeptide, wherein at least one of the Fc polypeptide monomers is linked to an IDS enzyme sequence. For example, in certain embodiments, the protein comprises a fusion polypeptide comprising a first Fc polypeptide linked to the IDS amino acid sequence; a second Fc polypeptide that forms an Fc dimer with the first Fc polypeptide; and a transferrin receptor (TfR) binding domain.

In some aspects, a protein described herein comprises: (i) a first Fc polypeptide, which may contain modifications (e.g., one or more modifications that promote heterodimerization) or may be a wild-type Fc polypeptide; and an IDS enzyme, IDS enzyme variant, or a catalytically active fragment thereof; and (ii) a second Fc polypeptide, which may contain modifications (e.g., one or more modifications that promote heterodimerization) or may be a wild-type Fc polypeptide; and optionally an IDS enzyme, IDS enzyme variant, or a catalytically active fragment thereof. In some embodiments, the first Fc polypeptide and/or the second Fc polypeptide does not include an immunoglobulin heavy and/or light chain variable region sequence or an antigen-binding portion thereof. In certain embodiments, the protein does not comprise an immunoglobulin heavy and/or light chain variable region sequence or an antigen-binding portion thereof.

In some embodiments, the first and/or the second Fc polypeptide comprises modifications that result in binding to a TfR (i.e., the Fc polypeptide is modified to comprise the TfR binding domain). These proteins may be referenced herein as an enzyme transport vehicle (ETV) in conjunction with an iduronate 2-sulfatase (IDS) enzyme, or ETV:IDS. ETV:IDS proteins, which may be used in a method described herein, are discussed below and are described in WO 2019/070577, which is incorporated by reference herein for all purposes. A particular example of ETV:IDS is ETV:IDS 35.23.2 as described herein.

An IDS enzyme sequence incorporated into the protein described herein is catalytically active, i.e., it retains the enzymatic activity that is deficient in Hunter syndrome. In some embodiments, a protein as described herein comprises a full-length IDS wild-type sequence. In some embodiments, a protein as described herein comprises a mature IDS wild-type sequence. In some embodiments, a protein as described herein comprises a catalytically active fragment or variant of a wild-type IDS sequence.

In some embodiments, the IDS amino acid sequence comprises an amino acid sequence having at least 80%, at least 85%, at least 90%, at least 95% identity, at least 96% identity, at least 97% identity, at least 98% identity, or at least 99% identity to the amino acid sequence of any one of SEQ ID NOs: 4, 5, 6, 7, and 8, or comprises the amino acid sequence of any one of SEQ ID NOs: 4, 5, 6, 7, and 8. In some embodiments, the IDS amino acid sequence comprises an amino acid sequence having at least 80%, at least 85%, at least 90%, at least 95% identity, at least 96% identity, at least 97% identity, at least 98% identity, or at least 99% identity to the amino acid sequence of SEQ ID NO: 6, or comprises the amino acid sequence of SEQ ID NO: 6. In some embodiments, the IDS amino acid sequence comprises an amino acid sequence having at least 80%, at least 85%, at least 90%, at least 95% identity, at least 96% identity, at least 97% identity, at least 98% identity, or at least 99% identity to the amino acid sequence of SEQ ID NO: 7, or comprises the amino acid sequence of SEQ ID NO: 7. In some embodiments, the IDS amino acid sequence comprises an amino acid sequence having at least 80%, at least 85%, at least 90%, at least 95% identity, at least 96% identity, at least 97% identity, at least 98% identity, or at least 99% identity to the amino acid sequence of SEQ ID NO: 8, or comprises the amino acid sequence of SEQ ID NO: 8.

As discussed above, in some embodiments, the IDS enzyme is a catalytically active variant or a catalytically active fragment of an IDS protein or IDS variant (e.g., comprises an IDS amino acid sequence described herein). In some embodiments, a catalytically active variant or catalytically active fragment of an IDS enzyme or IDS enzyme variant has at least 50%, at least 55%, at least 60%, at least 65%, at least 70%, at least 75%, at least 80%, at least 85%, at least 90%, at least 95%, or greater of the activity of the wild-type IDS enzyme.

In some embodiments, an IDS enzyme, IDS enzyme variant, or a catalytically active fragment thereof, that is present in a protein described herein, retains at least 25% of its activity compared to its activity when not joined to an Fc polypeptide or a TfR binding domain (e.g. a TfR binding Fc polypeptide). In some embodiments, an IDS enzyme, IDS enzyme variant, or a catalytically active fragment thereof, retains at least 10%, or at least 15%, 20%, 25%, 30%, 35%, 40%, 45%, 50%, 55%, 60%, 65%, 70%, 75%, 80%, 85%, 90%, or 95%, of its activity compared to its activity when not joined to an Fc polypeptide or a TfR binding domain (e.g. a TfR binding Fc polypeptide). In some embodiments, an IDS enzyme, IDS enzyme variant, or a catalytically active fragment thereof, retains at least 80%, 85%, 90%, or 95% of its activity compared to its activity when not joined to an Fc polypeptide or a TfR binding domain (e.g., a TfR binding Fc polypeptide). In some embodiments, fusion to an Fc polypeptide does not decrease the activity of the IDS enzyme, IDS enzyme variant, or catalytically active fragment thereof. In some embodiments, fusion to a TfR binding domain (e.g. a TfR binding Fc polypeptide) does not decrease the activity of the IDS enzyme, IDS enzyme variant, or catalytically active fragment thereof.

### Fc Polypeptide Modifications

A protein as described herein may comprise two Fc polypeptides, wherein one or both of the Fc polypeptides are wild-type Fc polypeptides, e.g., human IgG1 Fc polypeptides. However, in certain embodiments, an Fc polypeptide incorporated in a protein described herein may comprise certain modifications. For example, an Fc polypeptide may comprise modifications that result in binding to a TfR (i.e., the Fc polypeptide is modified to comprise a TfR binding domain described herein). Additionally, an Fc polypeptide may comprise other modifications, such as modifications that promote heterodimerization, increase serum stability or serum half-life, modulate effector function, influence glycosylation, and/or reduce immunogenicity in humans. Examples of various modifications that may be included in an Fc polypeptide are described in WO2019/070577, which is incorporated by reference herein in its entirety for all purposes.

Thus, in certain embodiments, a protein described herein comprises two Fc polypeptides, wherein 1) one Fe is a wild-type Fc polypeptide, e.g., a human IgG1 Fc polypeptide, and the other Fe is a modified Fc polypeptide; or 2) both Fc polypeptides each comprise independently selected modifications (e.g., a modification described herein). For example, in certain embodiments, a protein described herein comprises two Fc polypeptides, wherein one Fe is a wild-type Fc polypeptide, e.g., a human IgG1 Fc polypeptide; and the other Fe is modified to bind to a TfR, and optionally further comprises one or more additional modifications. In certain other embodiments, both Fc polypeptides each comprise independently selected modifications (e.g., a modification described herein). For example, in certain embodiments, a protein described herein comprises two Fc polypeptides, wherein one Fe is not modified to bind to a TfR but comprises one or more other modifications described herein; and the other Fe is modified to bind to a TfR, and optionally further comprises one or more additional modifications. In certain other embodiments, a protein described herein comprises two Fe polypeptides, wherein both Fc polypeptides are modified to bind to a TfR, and optionally further comprise one or more additional modifications.

Amino acid residues designated in various Fc modifications, including those introduced in a modified Fc polypeptide that binds to a TfR, are numbered herein using EU index numbering. Any Fc polypeptide, e.g., an IgG1, IgG2, IgG3, or IgG4 Fc polypeptide, may have modifications, e.g., amino acid substitutions, in one or more positions as described herein.

A modified (e.g., enhancing heterodimerization and/or TfR binding) Fc polypeptide present in a protein described herein can have at least 70% identity, at least 75% identity, at least 80% identity, at least 85% identity, at least 90% identity, at least 95% identity, at least 96% identity, at least 97% identity, at least 98% identity, or at least 99% identity to a native Fc region sequence or a fragment thereof, e.g., a fragment of at least 50 amino acids or at least 100 amino acids, or greater in length. In some embodiments, the native Fe amino acid sequence is the Fc region sequence of SEQ ID NO:1. In some embodiments, the native Fe amino acid sequence is the Fc region sequence of SEQ ID NO:42. In some embodiments, the modified Fc polypeptide has at least 70% identity, at least 75% identity, at least 80% identity, at least 85% identity, at least 90% identity, at least 95% identity, at least 96% identity, at least 97% identity, at least 98% identity, or at least 99% identity to amino acids 1-110 of SEQ ID NO:1; to amino acids 1-110 of SEQ ID NO:42; to amino acids 111-217 of SEQ ID NO:1, to amino acids 111-216 of SEQ ID NO:42, or a fragment thereof, e.g., a fragment of at least 50 amino acids or at least 100 amino acids, or greater in length.

### Fc Polypeptide Modifications for TfR Binding

In some aspects, provided herein are proteins that comprise a TfR binding domain and are capable of being transported across the blood-brain barrier (BBB). In some embodiments a protein described herein specifically binds to TfR. In certain embodiments, the protein binds to TfR with an affinity of from about 1 nM to about 1 μM. In certain embodiments, the protein binds to TfR with an affinity of from about 1 nM to about 500 nM. In certain embodiments, the protein binds to TfR with an affinity of from about 50 nM to about 350 nM. In certain embodiments, the protein binds to TfR with an affinity of about 50 nM, about 60 nM, about 70 nM, about 80 nM, about 90 nM, about 100 nM, about 110 nM, about 120 nM, about 130 nM, about 140 nM, about 150 nM, about 160 nM, about 170 nM, about 180 nM, about 190 nM, about 200 nM, about 210 nM, about 220 nM, about 230 nM, about 240 nM, about 250 nM, about 275 nM, about 300 nM, about 325 nM, or about 350 nM.

In certain embodiments, the protein comprises an Fc polypeptide that has been modified to comprise a TfR binding domain. In some embodiments, a modified Fc polypeptide that specifically binds to TfR comprises substitutions in a CH3 domain (i.e., the CH3 domain has been modified to comprise a TfR binding domain). In some embodiments, a modified Fc polypeptide comprises a human Ig CH3 domain, such as an IgG CH3 domain, that is modified for TfR binding activity. The CH3 domain can be of any IgG subtype, i.e., from IgG1, IgG2, IgG3, or IgG4. In the context of IgG antibodies, a CH3 domain refers to the segment of amino acids from about position 341 to about position 447 as numbered according to the EU numbering scheme.

In some embodiments, a modified Fc polypeptide present in a protein described herein comprises at least one, two, or three substitutions; and in some embodiments, at least four, five, six, seven, eight, or nine substitutions at amino acid positions 384, 386, 387, 388, 389, 390, 413, 416, and 421, according to the EU numbering scheme. In some embodiments, a modified Fc polypeptide present in a protein described herein comprises substitutions at amino acid positions 384, 386, 387, 388, 389, 390, 413, 416, and 421, according to the EU numbering scheme.

In certain embodiments, the modified Fc polypeptide comprises two, three, four, five, six, seven, eight, or nine positions selected from the following: Tyr or Phe at position 384; Thr at position 386; Glu at position 387; Trp at position 388; Ser, Ala, Val, or Asn at position 389; Thr or Ser at position 413; Glu or Ser at position 415; Glu at position 416; and/or Phe at position 421. In some embodiments, the modified Fc polypeptide comprises amino acid residues at all nine positions as follows: Tyr or Phe at position 384; Thr at position 386; Glu at position 387; Trp at position 388; Ser, Ala, Val, or Asn at position 389; Thr or Ser at position 413; Glu or Ser at position 415; Glu at position 416; and/or Phe at position 421.

In certain embodiments, the modified Fc polypeptide comprises two, three, four, five, six, seven, eight, nine, ten, or eleven positions selected from the following: Trp, Leu, or Glu at position 380; Tyr or Phe at position 384; Thr at position 386; Glu at position 387; Trp at position 388; Ser, Ala, Val, or Asn at position 389; Ser or Asn at position 390; Thr or Ser at position 413; Glu or Ser at position 415; Glu at position 416; and/or Phe at position 421. In some embodiments, the modified Fc polypeptide comprises all eleven positions as follows: Trp, Leu, or Glu at position 380; Tyr or Phe at position 384; Thr at position 386; Glu at position 387; Trp at position 388; Ser, Ala, Val, or Asn at position 389; Ser or Asn at position 390; Thr or Ser at position 413; Glu or Ser at position 415; Glu at position 416; and/or Phe at position 421.

In some embodiments, a modified Fc polypeptide that specifically binds to TfR comprises Ala or Ser at position 389, according to EU numbering. In some embodiments, a modified Fc polypeptide that specifically binds to TfR comprises at the following positions, according to EU numbering: Trp, Glu or Leu at position 380; Ala or Ser at position 389; and Asn or Ser at position 390. In some embodiments, a modified Fc polypeptide that specifically binds to TfR comprises at the following positions, according to EU numbering: Trp, Glu or Leu at position 380; Tyr at position 384; Thr at position 386; Glu at position 387; Trp at position 388; Ala or Ser at position 389; Asn or Ser at position 390; Thr at position 413; Glu at position 415; Glu at position 416; and Phe at position 421.

In some embodiments, a modified Fc polypeptide that specifically binds to TfR comprises Ala at position 389, according to EU numbering. In some embodiments, a modified Fc polypeptide that specifically binds to TfR comprises at the following positions, according to EU numbering: Glu at position 380; Ala at position 389; and Asn at position 390. In some embodiments, a modified Fc polypeptide that specifically binds to TfR comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421.

In some embodiments, the modified Fc polypeptide has at least 70% identity, at least 75% identity, at least 80% identity, at least 85% identity, at least 90% identity, at least 95% identity, at least 96% identity, at least 97% identity, at least 98% identity, or at least 99% identity to amino acids 111-217 of any one of SEQ ID NOS:11-17, 19-20, 22-24, and 26-28 (e.g., SEQ ID NOS:11-14). In some embodiments, the modified Fc polypeptide has at least 70% identity, at least 75% identity, at least 80% identity, at least 85% identity, at least 90% identity, at least 95% identity, at least 96% identity, at least 97% identity, at least 98% identity, or at least 99% identity to amino acids 111-216 of any one of SEQ ID NOS: 43-46, 48-50, 52-55, and 57-60 (e.g., SEQ ID NOS: 43, 48, 52 and 57). In some embodiments, the modified Fc polypeptide has at least 70% identity, at least 75% identity, at least 80% identity, at least 85% identity, at least 90% identity, at least 95% identity, at least 96% identity, at least 97% identity, at least 98% identity, or at least 99% identity to any one of SEQ ID NOS: 11-17, 19-20, 22-24, 26-28, 43-46, 48-50, 52-55, and 57-60 (e.g., SEQ ID NOS:11-14, 43, 48, 52, and 57).

In some embodiments, the modified Fc polypeptide has at least 75% identity, at least 80% identity, at least 85% identity, at least 90% identity, at least 95% identity, at least 96% identity, at least 97% identity, at least 98% identity, or at least 99% identity to any one of SEQ ID NOS: 11-17, 19-20, 22-24, 26-28, 43-46, 48-50, 52-55, and 57-60 (e.g., SEQ ID NOS: 11-14, 43, 48, 52, and 57), and further comprises at least five, six, seven, eight, nine, ten, eleven, twelve, thirteen, fourteen, fifteen, or sixteen of the positions, numbered according to the EU index, as follows: Trp, Tyr, Leu, Gln, or Glu at position 380; Leu, Tyr, Met, or Val at position 384; Leu, Thr, His, or Pro at position 386; Val, Pro, or an acidic amino acid at position (e.g., Glu) 387; an aromatic amino acid, e.g., Trp, at position 388; Val, Ser, or Ala at position 389; Ser or Asn at position 390; Ser, Thr, Gln, or Phe at position 391; Gln, Phe, or His at position 392; an acidic amino acid, Ala, Ser, Leu, Thr, or Pro at position 413; Lys, Arg, Gly or Pro at position 414; Glu or Ser at position 415; Thr or an acidic amino acid (e.g., Glu) at position 416; Trp, Tyr, His or Phe at position 421; Ser, Thr, Glu or Lys at position 424; and Ser, Trp, or Gly at position 426.

In some embodiments, the modified Fc polypeptide comprises the amino acid sequence of any one of SEQ ID NOS: 11-14, 43, 48, 52, and 57. In other embodiments, the modified Fc polypeptide comprises the amino acid sequence of any one of SEQ ID NOS: 11-14, 43, 48, 52, and 57, but in which one, two, or three amino acids are substituted.

### Other Fc Polypeptide Modifications

In some aspects, a protein described herein comprises two Fc polypeptides that may each comprise independently selected modifications or may be a wild-type Fc polypeptide, e.g., a human IgG1 Fc polypeptide. In some aspects, a protein described herein comprises two Fc polypeptides, wherein one or both Fc polypeptides each comprise independently selected modifications (e.g., a modification described herein). Non-limiting examples of other mutations that can be introduced into one or both Fc polypeptides include, e.g., mutations to increase serum stability or serum half-life, to modulate effector function, to influence glycosylation, to reduce immunogenicity in humans, and/or to provide for knob and hole heterodimerization of the Fc polypeptides. Examples of various modifications that may be included in an Fc polypeptide are described in WO2019/070577, which is incorporated by reference herein in its entirety for all purposes.

In some embodiments, the Fc polypeptides present in the protein independently have an amino acid sequence identity of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% to a corresponding wild-type Fc polypeptide (e.g., a human IgG1, IgG2, IgG3, or IgG4 Fc polypeptide).

In some embodiments, the Fc polypeptides present in the protein include knob and hole mutations to promote heterodimer formation and hinder homodimer formation. Generally, the modifications introduce a protuberance (“knob”) at the interface of one polypeptide and a corresponding cavity (“hole”) in the interface of another polypeptide, such that the protuberance can be positioned in the cavity so as to promote heterodimer formation and thus hinder homodimer formation. Protuberances are constructed by replacing small amino acid side chains from the interface of one polypeptide with larger side chains (e.g., tyrosine or tryptophan). Compensatory cavities of identical or similar size to the protuberances are created in the interface of the other polypeptide by replacing large amino acid side chains with smaller ones (e.g., alanine or threonine). In some embodiments, such additional mutations are at a position in the Fc polypeptide that does not have a negative effect on binding of the polypeptide to a TfR.

In one illustrative embodiment of a knob and hole approach for dimerization, position 366 (numbered according to the EU numbering scheme) of one of the Fc polypeptides present in the protein comprises a tryptophan in place of a native threonine. The other Fc polypeptide in the dimer has a valine at position 407 (numbered according to the EU numbering scheme) in place of the native tyrosine. The other Fc polypeptide may further comprise a substitution in which the native threonine at position 366 (numbered according to the EU numbering scheme) is substituted with a serine and a native leucine at position 368 (numbered according to the EU numbering scheme) is substituted with an alanine. Thus, one of the Fc polypeptides of a protein described herein has the T366W knob mutation and the other Fc polypeptide has the Y407V mutation, which is typically accompanied by the T366S and L368A hole mutations. In certain embodiments, the first Fc polypeptide contains the T366S, L368A, and Y407V substitutions and the second Fc polypeptide contains the T366W substitution. In certain other embodiments, the first Fc polypeptide contains the T366W substitution and the second Fc polypeptide contains the T366S, L368A, and Y407V substitutions.

In some embodiments, one or both Fc polypeptides present in a protein described herein may comprise modifications that reduce effector function, i.e., having a reduced ability to induce certain biological functions upon binding to an Fc receptor expressed on an effector cell that mediates the effector function. Examples of effector functions include, but are not limited to, C1q binding and complement dependent cytotoxicity (CDC), Fc receptor binding, antibody-dependent cell-mediated cytotoxicity (ADCC), antibody-dependent cell-mediated phagocytosis (ADCP), down-regulation of cell surface receptors (e.g., B cell receptor), and B-cell activation. Effector functions may vary with the antibody class. For example, native human IgG1 and IgG3 antibodies can elicit ADCC and CDC activities upon binding to an appropriate Fc receptor present on an immune system cell; and native human IgG1, IgG2, IgG3, and IgG4 can elicit ADCP functions upon binding to the appropriate Fc receptor present on an immune cell.

In some embodiments, one or both Fc polypeptides present in a protein described herein may also be engineered to contain other modifications for heterodimerization, e.g., electrostatic engineering of contact residues within a CH3-CH3 interface that are naturally charged or hydrophobic patch modifications.

In some embodiments, one or both Fc polypeptides present in a protein described herein may include additional modifications that modulate effector function.

In some embodiments, one or both Fc polypeptides present in a protein described herein may comprise modifications that reduce or eliminate effector function. Illustrative Fc polypeptide mutations that reduce effector function include, but are not limited to, substitutions in a CH2 domain, e.g., at positions 234 and 235, according to the EU numbering scheme. For example, in some embodiments, one or both Fc polypeptides can comprise alanine residues at positions 234 and 235. Thus, one or both Fc polypeptides may have L234A and L235A (LALA) substitutions.

Additional Fc polypeptide mutations that modulate an effector function include, but are not limited to, the following: position 329 may have a mutation in which proline is substituted with a glycine, serine or arginine or an amino acid residue large enough to destroy the Fc/Fcγ receptor interface that is formed between proline 329 of the Fc and tryptophan residues Trp 87 and Trp 110 of FcγRIII. Additional illustrative substitutions include S228P, E233P, L235E, N297A, N297D, and P331S, according to the EU numbering scheme. Multiple substitutions may also be present, e.g., L234A and L235A of a human IgG1 Fc region; L234A, L235A, and P329G of a human IgG1 Fc region; L234A, L235A, and P329S of a human IgG1 Fc region; S228P and L235E of a human IgG4 Fc region; L234A and G237A of a human IgG1 Fc region; L234A, L235A, and G237A of a human IgG1 Fc region; V234A and G237A of a human IgG2 Fc region; L235A, G237A, and E318A of a human IgG4 Fc region; and S228P and L236E of a human IgG4 Fc region, according to the EU numbering scheme. In some embodiments, one or both Fc polypeptides may have one or more amino acid substitutions that modulate ADCC, e.g., substitutions at positions 298, 333, and/or 334, according to the EU numbering scheme.

In some embodiments, the C′terminal Lys residue is removed or not present in an Fc polypeptide described herein (i.e., the Lys residue at position 447, according to the EU numbering scheme).

### Illustrative Fc Polypeptides Comprising Other Modifications, and Optionally Modifications for TfR Binding

By way of non-limiting example, one or both Fc polypeptides present in a protein described herein may comprise mutations including a knob mutation (e.g., T366W as numbered according to the EU numbering scheme), hole mutations (e.g., T366S, L368A, and Y407V as numbered according to the EU numbering scheme), mutations that modulate effector function (e.g., L234A, L235A, and/or P329G or P329S (e.g., L234A and L235A; L234A, L235A, and P329G; or L234A, L235A, and P329S) as numbered according to the EU numbering scheme), and/or mutations that increase serum stability or serum half-life (e.g., (i) M252Y, S254T, and T256E as numbered with reference to EU numbering, or (ii) N434S with or without M428L as numbered according to the EU numbering scheme). Further, in certain embodiments, the C′terminal Lys residue may not be present. By way of illustration, SEQ ID NOS:15-31, 44-47, 49-51, 53-56, and 58-63 provide non-limiting examples of modified Fc polypeptides comprising one or more of these mutations (e.g., SEQ ID NOS:15-29, 44-47, 49-51, 53-56, and 58-61 are capable of specifically binding to TfR and comprise one or more of these additional mutations).

In some embodiments, an Fc polypeptide, which may be modified or unmodified for TfR binding, comprises a knob mutation. In some embodiments, an Fc polypeptide comprises a sequence having a knob mutation (e.g., T366W as numbered according to the EU numbering scheme) and at least 85% identity, at least 90% identity, at least 95% identity, at least 96% identity, at least 97% identity, at least 98% identity, or at least 99% identity to the sequence of any one of SEQ ID NOS:1, 11-14, 42-43, 48, 52, and 57. For example, in some embodiments, an Fc polypeptide having the sequence of any one of SEQ ID NOS: 1, 11-14, 42-43, 48, 52, and 57 may be modified to have a knob mutation. In certain embodiments, the Fc polypeptide comprises a knob mutation and a sequence having at least 85% identity, at least 90% identity, at least 95% identity, at least 96% identity, at least 97% identity, at least 98% identity, or at least 99% identity to the sequence of any one of SEQ ID NOS:15, 22, 26, 44, 53, and 58, or comprises the sequence of any one of SEQ ID NOS: 15, 22, 26, 44, 53, and 58.

In some embodiments, a Fc polypeptide, which may be modified or unmodified for TfR binding, comprises a knob mutation (e.g., T366W as numbered according to the EU numbering scheme) and mutations that modulate effector function (e.g., L234A, L235A, and/or P329G or P329S (e.g., L234A and L235A; L234A, L235A, and P329G; or L234A, L235A, and P329S) as numbered according to the EU numbering scheme). In some embodiments, an Fc polypeptide comprises a sequence having a knob mutation (e.g., T366W as numbered according to the EU numbering scheme), mutations that modulate effector function (e.g., L234A, L235A, and/or P329G or P329S (e.g., L234A and L235A; L234A, L235A, and P329G; or L234A, L235A, and P329S) as numbered according to the EU numbering scheme), and at least 85% identity, at least 90% identity, at least 95% identity, at least 96% identity, at least 97% identity, at least 98% identity, or at least 99% identity to the sequence of any one of SEQ ID NOS: 1, 11-14, 42-43, 48, 52, and 57. For example, in some embodiments, an Fc polypeptide having the sequence of any one of SEQ ID NOS: 1, 11-14, 42-43, 48, 52, and 57 may be modified to have a knob mutation and mutations that modulate effector function. In certain embodiments, the Fc polypeptide comprises a knob mutation and mutations that modulate effector function, and a sequence having at least 85% identity, at least 90% identity, at least 95% identity, at least 96% identity, at least 97% identity, at least 98% identity, or at least 99% identity to the sequence of any one of SEQ ID NOS:16-21, 23-25, 27-29, 45-47, 49-51, 54-56, and 59-61, or comprises the sequence of any one of SEQ ID NOS: 16-21, 23-25, 27-29, 45-47, 49-51, 54-56, and 59-61.

In some embodiments, a Fc polypeptide, which may be modified or unmodified for TfR binding, comprises hole mutations (e.g., T366S, L368A, and Y407V as numbered according to the EU numbering scheme). In some embodiments, an Fc polypeptide comprises a sequence having hole mutations (e.g., T366S, L368A, and Y407V as numbered according to the EU numbering scheme) and at least 85% identity, at least 90% identity, at least 95% identity, at least 96% identity, at least 97% identity, at least 98% identity, or at least 99% identity to the sequence of any one of SEQ ID NOS: 1, 11-14, 42-43, 48, 52, and 57. For example, in some embodiments, an Fc polypeptide having the sequence of any one of SEQ ID NOS: 1, 11-14, 42-43, 48, 52, and 57 may be modified to have hole mutations. In certain embodiments, the Fe polypeptide comprises a hole mutation and a sequence having at least 85% identity, at least 90% identity, at least 95% identity, at least 96% identity, at least 97% identity, at least 98% identity, or at least 99% identity to the sequence of any one of SEQ ID NOS:30 and 62, or comprises the sequence of any one of SEQ ID NOS:30 and 62.

In some embodiments, a Fc polypeptide, which may be modified or unmodified for TfR binding, comprises hole mutations (e.g., T366S, L368A, and Y407V as numbered according to the EU numbering scheme) and mutations that modulate effector function (e.g., L234A, L235A, and/or P329G or P329S (e.g., L234A and L235A; L234A, L235A, and P329G; or L234A, L235A, and P329S) as numbered according to the EU numbering scheme). In some embodiments, an Fc polypeptide comprises a sequence having hole mutations (e.g., T366S, L368A, and Y407V as numbered according to the EU numbering scheme), mutations that modulate effector function (e.g., L234A, L235A, and/or P329G or P329S (e.g., L234A and L235A; L234A, L235A, and P329G; or L234A, L235A, and P329S) as numbered according to the EU numbering scheme), and at least 85% identity, at least 90% identity, at least 95% identity, at least 96% identity, at least 97% identity, at least 98% identity, or at least 99% identity to the sequence of any one of SEQ ID NOS: 1, 11-14, 42-43, 48, 52, and 57. For example, in some embodiments, an Fc polypeptide having the sequence of any one of SEQ ID NOS: 1, 11-14, 42-43, 48, 52, and 57 may be modified to have hole mutations and mutations that modulate effector function. In certain embodiments, the Fc polypeptide comprises hole mutations and mutations that modulate effector function and a sequence having at least 85% identity, at least 90% identity, at least 95% identity, at least 96% identity, at least 97% identity, at least 98% identity, or at least 99% identity to the sequence of any one of SEQ ID NOS:31 and 63, or comprises the sequence of any one of SEQ ID NOS:31 and 63.

### IDS Enzymes Linked to Fc Polypeptides

In some embodiments, a protein described herein comprises two Fc polypeptides as described herein and one or both of the Fc polypeptides may further comprise a partial or full hinge region. The hinge region can be from any immunoglobulin subclass or isotype. An illustrative immunoglobulin hinge is an IgG hinge region, such as an IgG1 hinge region, e.g., human IgG1 hinge amino acid sequence EPKSCDKTHTCPPCP (SEQ ID NO:9) or a portion thereof (e.g., DKTHTCPPCP; SEQ ID NO:10). In some embodiments, the hinge region is at the N-terminal region of the Fc polypeptide.

In certain embodiments, the N-terminus of the first Fc polypeptide is linked to the IDS enzyme, IDS enzyme variant, or catalytically active fragment thereof. In certain embodiments, the C-terminus of the first Fc polypeptide is linked to the IDS enzyme, IDS enzyme variant, or catalytically active fragment thereof.

In certain embodiments, a protein described herein comprises a single IDS enzyme, IDS enzyme variant, or catalytically active fragment thereof.

In certain other embodiments, a protein as described herein comprises a second IDS enzyme, IDS enzyme variant, or catalytically active fragment thereof. For example, in certain embodiments, the second Fc polypeptide is linked to an IDS enzyme, IDS enzyme variant, or catalytically active fragment thereof. In certain embodiments, the N-terminus of the second Fc polypeptide is linked to the second IDS enzyme, IDS enzyme variant, or catalytically active fragment thereof. In certain embodiments, the C-terminus of the second Fc polypeptide is linked the second IDS enzyme, IDS enzyme variant, or catalytically active fragment thereof.

In certain embodiments, the N-terminus of the first Fc polypeptide is linked to a first IDS enzyme, IDS enzyme variant, or catalytically active fragment thereof; and the N-terminus of a second Fc polypeptide is linked to a second IDS enzyme, IDS enzyme variant, or catalytically active fragment thereof.

In certain embodiments, the C-terminus of the first Fc polypeptide is linked to a first IDS enzyme, IDS enzyme variant, or catalytically active fragment thereof, and the C-terminus of a second Fc polypeptide is linked to a second IDS enzyme, IDS enzyme variant, or catalytically active fragment thereof.

In certain embodiments, the N-terminus of the first Fc polypeptide is linked to a first IDS enzyme, IDS enzyme variant, or catalytically active fragment thereof; and the C-terminus of a second Fc polypeptide is linked to a second IDS enzyme, IDS enzyme variant, or catalytically active fragment thereof.

In certain embodiments, the C-terminus of the first Fc polypeptide is linked to a first IDS enzyme, IDS enzyme variant, or catalytically active fragment thereof, and the N-terminus of a second Fc polypeptide is linked to a second IDS enzyme, IDS enzyme variant, or catalytically active fragment thereof.

In some embodiments, an Fc polypeptide is joined to the IDS enzyme by a linker, e.g., a peptide linker. In some embodiments, the Fc polypeptide is joined to the IDS enzyme by a peptide bond or by a peptide linker, e.g., is a fusion polypeptide. The peptide linker may be configured such that it allows for the rotation of the IDS enzyme relative to the Fc polypeptide to which it is joined; and/or is resistant to digestion by proteases. Peptide linkers may contain natural amino acids, unnatural amino acids, or a combination thereof. In some embodiments, the peptide linker may be a flexible linker, e.g., containing amino acids such as Gly, Asn, Ser, Thr, Ala, and the like. Such linkers are designed using known parameters and may be of any length and contain any number of repeat units of any length (e.g., repeat units of Gly and Ser residues). For example, the linker may have repeats, such as two, three, four, five, or more Gly₄-Ser (SEQ ID NO:40) repeats or a single Gly₄-Ser (SEQ ID NO:40). In some embodiments, the peptide linker may include a protease cleavage site, e.g., that is cleavable by an enzyme present in the central nervous system.

In some embodiments, the IDS enzyme is joined to the N-terminus of the Fc polypeptide, e.g., by a Gly₄-Ser linker (SEQ ID NO:40) or a (Gly₄-Ser)₂ linker (SEQ ID NO:41). In some embodiments, the Fc polypeptide may comprise a hinge sequence or partial hinge sequence at the N-terminus that is joined to the linker or directly joined to the IDS enzyme.

In some embodiments, the IDS enzyme is joined to the C-terminus of the Fc polypeptide, e.g., by a Gly₄-Ser linker (SEQ ID NO:40) or a (Gly₄-Ser)₂ linker (SEQ ID NO:41). In some embodiments, the C-terminus of the Fc polypeptide is directly joined to the IDS enzyme.

In some embodiments, the IDS enzyme is joined to the Fc polypeptide by a chemical cross-linking agent. Such conjugates can be generated using well-known chemical cross-linking reagents and protocols. For example, there are a large number of chemical cross-linking agents that are known to those skilled in the art and useful for cross-linking the polypeptide with an agent of interest. For example, the cross-linking agents are heterobifunctional cross-linkers, which can be used to link molecules in a stepwise manner. Heterobifunctional cross-linkers provide the ability to design more specific coupling methods for conjugating proteins, thereby reducing the occurrences of unwanted side reactions such as homo-protein polymers. A wide variety of heterobifunctional cross-linkers are known in the art, including N-hydroxysuccinimide (NHS) or its water soluble analog N-hydroxysulfosuccinimide (sulfo-NHS), succinimidyl 4-(N-maleimidomethyl)cyclohexane-1-carboxylate (SMCC), m-maleimidobenzoyl-N-hydroxysuccinimide ester (MBS); N-succinimidyl (4-iodoacetyl) aminobenzoate (SIAB), succinimidyl 4-(p-maleimidophenyl)butyrate (SMPB), 1-ethyl-3-(3-dimethylaminopropyl)carbodiimide hydrochloride (EDC); 4-succinimidyloxycarbonyl-a-methyl-a-(2-pyridyldithio)-toluene (SMPT), N-succinimidyl 3-(2-pyridyldithio)propionate (SPDP), and succinimidyl 6-[3-(2-pyridyldithio)propionate]hexanoate (LC-SPDP). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives so as to reduce the amount of linker cleavage in vivo. In addition to the heterobifunctional cross-linkers, there exist a number of other cross-linking agents including homobifunctional and photoreactive cross-linkers. Disuccinimidyl subcrate (DSS), bismaleimidohexane (BMH) and dimethylpimelimidate. 2HCl (DMP) are examples of useful homobifunctional cross-linking agents, and bis-[B-(4-azidosalicylamido)ethyl]disulfide (BASED) and N-succinimidyl-6(4′-azido-2′-nitrophenylamino)hexanoate (SANPAH) are examples of useful photoreactive cross-linkers.

### Illustrative Proteins Comprising an IDS Enzyme

In some aspects, a protein described herein comprises a first Fc polypeptide that is linked to an IDS enzyme, IDS enzyme variant, or a catalytically active fragment thereof; and a second Fc polypeptide; wherein the first and/or second Fc polypeptide is a modified Fc that is capable of binding (e.g., specifically binding) to a TfR (i.e., the Fc comprises a TfR binding domain described herein). In certain embodiments, the second Fc polypeptide forms an Fc dimer with the first Fc polypeptide. In some embodiments, the first Fc polypeptide and/or the second Fc polypeptide does not include an immunoglobulin heavy and/or light chain variable region sequence or an antigen-binding portion thereof. In certain embodiments, a protein described herein comprises a single IDS enzyme, IDS enzyme variant, or a catalytically active fragment thereof. In some other aspects, the protein further comprises a second IDS enzyme, IDS enzyme variant, or a catalytically active fragment thereof (e.g., which may be linked to the second Fc polypeptide). The terms “ETV:IDS,” “ETV:IDS protein,” or “ETV:IDS fusion protein” can refer to any of the aforementioned embodiments comprising the first Fc polypeptide linked to an IDS enzyme, IDS enzyme variant, or a catalytically active fragment thereof, and the second Fc polypeptide, wherein the first and/or second Fc polypeptide is a modified Fc that is capable of binding (e.g., specifically binding) to a TfR.

In some embodiments, the first Fc polypeptide is a modified Fc polypeptide and/or the second Fc polypeptide is a modified Fc polypeptide (e.g., comprises one or more modifications described herein). For example, in some embodiments, a modified Fc polypeptide contains one or more modifications that promote its heterodimerization to the other Fc polypeptide. In some embodiments, a modified Fc polypeptide contains one or more modifications that reduce effector function. In some embodiments, a modified Fc polypeptide contains one or more modifications that extend serum half-life. In some embodiments, a modified Fc polypeptide comprises one or more modifications that confer binding to a TfR. In some embodiments, such an Fc polypeptide specifically binds to TfR.

In some embodiments, the first Fc polypeptide is a modified Fc polypeptide. In some embodiments, the second Fc polypeptide is a modified Fc polypeptide. In some embodiments, the first and the second Fc polypeptide are each a modified Fc polypeptide. In some embodiments, the first Fc polypeptide is a modified polypeptide but does not specifically bind to TfR; and the second Fc polypeptide is a modified polypeptide that is capable of specifically binding to TfR, and optionally, further comprises one or more further modifications described herein. In other embodiments, the first Fc polypeptide is a modified polypeptide that is capable of specifically binding to TfR, and optionally, further comprises one or more further modifications described herein; and the second Fc polypeptide is a modified polypeptide but does not specifically binding to TfR. In some embodiments, the first Fc polypeptide is a modified polypeptide that is capable of specifically binding to TfR, and optionally, further comprises one or more further modifications described herein; and the second Fc polypeptide is a modified polypeptide that is capable of specifically binding to TfR, and optionally, further comprises one or more further modifications described herein.

In some embodiments, a protein described herein comprises a first polypeptide chain that comprises a first Fc polypeptide comprising T366S, L368A, and Y407V (hole) substitutions linked to an IDS enzyme, IDS enzyme variant, or a catalytically active fragment thereof, and a second polypeptide chain that comprises a second Fc polypeptide that comprises a T366W (knob) substitution, wherein the first and/or second Fc polypeptide is a modified polypeptide that is capable of binding to TfR. In some embodiments, the first Fc polypeptide and/or the second Fc polypeptide further comprises L234A and L235A (LALA) substitutions. In some embodiments, the first Fc polypeptide and/or the second Fc polypeptide further comprises L234A, L235A, and P329G (LALAPG) substitutions or comprises L234A, L235A, and P329S (LALAPS) substitutions. In some embodiments, the first Fc polypeptide and/or the second Fc polypeptide further comprises M252Y, S254T, and T256E (YTE) substitutions. In some embodiments, the first Fc polypeptide and/or the second Fc polypeptide further comprises: 1) L234A and L235A (LALA) substitutions; L234A, L235A, and P329G (LALAPG) substitutions; or L234A, L235A, and P329S (LALAPS) substitutions; and 2) M252Y, S254T, and T256E (YTE) substitutions. In some embodiments, the first Fc polypeptide and/or the second Fc polypeptide comprises human IgG1 wild-type residues at positions 234, 235, 252, 254, 256, and 366.

In some embodiments, the second Fc polypeptide is a modified polypeptide that is capable of binding to TfR. In some embodiments, the first Fc polypeptide linked to an IDS enzyme, IDS enzyme variant, or a catalytically active fragment thereof, is not modified to bind to TfR. In some embodiments, the second Fc polypeptide comprises knob, LALA/LALAPG/LALAPS, and/or YTE mutations, and comprises a sequence having at least 85% identity, at least 90% identity, at least 95% identity, at least 96% identity, at least 97% identity, at least 98% identity, or at least 99% identity to any one of SEQ ID NOS: 15-17, 19-20, 22-24, 26-28, 44-46, 49-50, 53-55, and 58-60; or comprises the sequence of any one of SEQ ID NOS: 15-17, 19-20, 22-24, 26-28, 44-46, 49-50, 53-55, and 58-60. In some embodiments, the first Fc polypeptide comprises hole, LALA/LALAPG/LALAPS, and/or YTE mutations, and comprises a sequence having at least 85% identity, at least 90% identity, at least 95% identity, at least 96% identity, at least 97% identity, at least 98% identity, or at least 99% identity to any one of SEQ ID NOS:30-31 and 62-63; or comprises the sequence of any one of SEQ ID NOS: 30-31 and 62-63. In some embodiments, the second Fc polypeptide comprises any one of SEQ ID NOS: 15-17, 19-20, 22-24, 26-28, and the first Fc polypeptide comprises any one of SEQ ID NOS:30-31. In some embodiments, the second Fc polypeptide comprises any one of SEQ ID NOS: 44-46, 49-50, 53-55, and 58-60, and the first Fc polypeptide comprises any one of SEQ ID NOS: 62-63. In some embodiments, the N-terminus of the first Fc polypeptide and/or the second Fc polypeptide includes a portion of an IgG1 hinge region (e.g., DKTHTCPPCP; SEQ ID NO:10). In some embodiments, the second Fc polypeptide comprises a sequence having at least 85%, at least 90%, at least 95% identity, at least 96% identity, at least 97% identity, at least 98% identity, or at least 99% identity to any one of SEQ ID NOS:18, 21, 25, and 29, or comprises the sequence of any one of SEQ ID NOS: 18, 21, 25, and 29. In some embodiments, the second Fc polypeptide comprises a sequence having at least 85%, at least 90%, or at least 95% identity, at least 96% identity, at least 97% identity, at least 98% identity, or at least 99% identity to any one of SEQ ID NOS:47, 51, 56, and 61, or comprises the sequence of any one of SEQ ID NOS: 47, 51, 56, and 61.

In some embodiments, an IDS enzyme present in a protein described herein is linked to a first polypeptide chain that comprises a first Fc polypeptide comprising a sequence having at least 85%, at least 90%, at least 95% identity, at least 96% identity, at least 97% identity, at least 98% identity, or at least 99% identity to any one of SEQ ID NOS: 30-31 and 62-63, or comprises the sequence of any one of SEQ ID NOS: 30-31 and 62-63 (e.g., as a fusion polypeptide). In some embodiments, the IDS enzyme is linked to the first Fc polypeptide by a linker, such as a flexible linker, and/or a hinge region or portion thereof (e.g., DKTHTCPPCP; SEQ ID NO: 10). In some embodiments, the N-terminus of the first Fc polypeptide includes a portion of an IgG1 hinge region (e.g., DKTHTCPPCP; SEQ ID NO:10). In some embodiments, the IDS enzyme comprises a sequence having at least 85%, at least 90%, at least 95% identity, at least 96% identity, at least 97% identity, at least 98% identity, or at least 99% identity to any one of SEQ ID NOS:6-8, or comprises the sequence of any one of SEQ ID NOS: 6-8. In some embodiments, the IDS sequence linked to the first Fc polypeptide comprises a sequence having at least 85%, at least 90%, at least 95% identity, at least 96% identity, at least 97% identity, at least 98% identity, or at least 99% identity to any one of SEQ ID NOS:32-37 and 64-69, or comprises the sequence of any one of SEQ ID NOS: 32-37 and 64-69. In some embodiments, the protein comprises a second Fc polypeptide comprising a sequence having at least 85%, at least 90%, at least 95% identity, at least 96% identity, at least 97% identity, at least 98% identity, or at least 99% identity to any one of SEQ ID NOS: 15-17, 19-20, 22-24, 26-28, 44-46, 49-50, 53-55, and 58-60, or comprises the sequence of any one of SEQ ID NOS: 15-17, 19-20, 22-24, 26-28, 44-46, 49-50, 53-55, and 58-60. In some embodiments, the N-terminus of the second Fc polypeptide includes a portion of an IgG1 hinge region (e.g., DKTHTCPPCP; SEQ ID NO:10). In some embodiments, the second Fc polypeptide comprises a sequence having at least 85%, at least 90%, at least 95% identity, at least 96% identity, at least 97% identity, at least 98% identity, or at least 99% identity to any one of SEQ ID NOS: 18, 21, 25, and 29, or comprises the sequence of any one of SEQ ID NOS: 18, 21, 25, and 29. In some embodiments, the second Fc polypeptide comprises a sequence having at least 85%, at least 90%, or at least 95% identity, at least 96% identity, at least 97% identity, at least 98% identity, or at least 99% identity to any one of SEQ ID NOS: 47, 51, 56, and 61, or comprises the sequence of any one of SEQ ID NOS: 47, 51, 56, and 61. In some embodiments, the IDS sequence linked to the first Fc polypeptide comprises the sequence of any one of SEQ ID NOS: 32-37; and the second Fc polypeptide comprises the sequence of any one of SEQ ID NOS: 18, 21, 25, and 29. In some embodiments, the IDS sequence linked to the first Fc polypeptide comprises the sequence of any one of SEQ ID NOS: 64-69; and the second Fc polypeptide comprises the sequence of any one of SEQ ID NOS: 47, 51, 56, and 61.

Accordingly, in certain embodiments, the protein comprises: a) a fusion polypeptide comprising a first Fc polypeptide linked to an iduronate 2-sulfatase (IDS) amino acid sequence, wherein the fusion polypeptide comprises SEQ ID NO: 35, 36, 37, 67, 68, or 69; and b) a second Fc polypeptide comprising SEQ ID NO: 29 or 56. The protein comprising any combination of the aforementioned sequences in a) and b) is also referred to as ETV:IDS 35.23.2 protein.

In certain embodiments, the fusion polypeptide comprises SEQ ID NO: 35; and the second Fc polypeptide comprises SEQ ID NO: 29. In certain embodiments, the fusion polypeptide is SEQ ID NO: 35; and the second Fc polypeptide is SEQ ID NO: 29.

In certain embodiments, the fusion polypeptide comprises SEQ ID NO: 67; and the second Fc polypeptide comprises SEQ ID NO: 56. In certain embodiments the fusion polypeptide is SEQ ID NO: 67; and the second Fc polypeptide is SEQ ID NO: 56.

In certain embodiments, the fusion polypeptide comprises SEQ ID NO: 36; and the second Fc polypeptide comprises SEQ ID NO: 29. In certain embodiments, the fusion polypeptide is SEQ ID NO: 36; and the second Fc polypeptide is SEQ ID NO: 29.

In certain embodiments, the fusion polypeptide comprises SEQ ID NO: 68; and the second Fc polypeptide comprises SEQ ID NO: 56. In certain embodiments, the fusion polypeptide is SEQ ID NO: 68; and the second Fc polypeptide is SEQ ID NO: 56.

In certain embodiments, the fusion polypeptide comprises SEQ ID NO: 37; and the second Fc polypeptide comprises SEQ ID NO: 29. In certain embodiments the fusion polypeptide is SEQ ID NO: 37; and the second Fc polypeptide is SEQ ID NO: 29.

In certain embodiments, the fusion polypeptide comprises SEQ ID NO: 69; and the second Fc polypeptide comprises SEQ ID NO: 56. In certain embodiments, the fusion polypeptide is SEQ ID NO: 69; and the second Fc polypeptide is SEQ ID NO: 56.

In certain embodiments, the protein comprises: a) a fusion polypeptide comprising a first Fc polypeptide linked to an iduronate 2-sulfatase (IDS) amino acid sequence, wherein the fusion polypeptide comprises SEQ ID NO: 35, 36, 37, 67, 68 or 69; and b) a second Fc polypeptide comprising SEQ ID NO: 25 or 61. The protein comprising any combination of the aforementioned sequences in a) and b) is also referred to as ETV:IDS 35.21.17.2 protein.

In certain embodiments, the fusion polypeptide comprises SEQ ID NO: 35; and the second Fc polypeptide comprises SEQ ID NO:25. In certain embodiments, the fusion polypeptide is SEQ ID NO: 35; and the second Fc polypeptide is SEQ ID NO: 25.

In certain embodiments, the fusion polypeptide comprises SEQ ID NO: 67; and the second Fc polypeptide comprises SEQ ID NO: 61. In certain embodiments the fusion polypeptide is SEQ ID NO: 67; and the second Fc polypeptide is SEQ ID NO: 61.

In certain embodiments, the fusion polypeptide comprises SEQ ID NO: 36; and the second Fc polypeptide comprises SEQ ID NO: 25. In certain embodiments, the fusion polypeptide is SEQ ID NO: 36; and the second Fc polypeptide is SEQ ID NO: 25.

In certain embodiments, the fusion polypeptide comprises SEQ ID NO: 68; and the second Fc polypeptide comprises SEQ ID NO: 61. In certain embodiments, the fusion polypeptide is SEQ ID NO: 68; and the second Fc polypeptide is SEQ ID NO: 61.

In certain embodiments, the fusion polypeptide comprises SEQ ID NO: 37; and the second Fc polypeptide comprises SEQ ID NO: 25. In certain embodiments the fusion polypeptide is SEQ ID NO: 37; and the second Fc polypeptide is SEQ ID NO: 25.

In certain embodiments, the fusion polypeptide comprises SEQ ID NO: 69; and the second Fc polypeptide comprises SEQ ID NO: 61. In certain embodiments, the fusion polypeptide is SEQ ID NO: 69; and the second Fc polypeptide is SEQ ID NO: 61.

In certain embodiments, the protein comprises: a) a fusion polypeptide comprising a first Fc polypeptide linked to an iduronate 2-sulfatase (IDS) amino acid sequence, wherein the fusion polypeptide comprises SEQ ID NO: 35, 36, 37, 67, 68 or 69; and b) a second Fc polypeptide comprising SEQ ID NO: 21 or 51. The protein comprising any combination of the aforementioned sequences in a) and b) is also referred to as ETV:IDS 35.21.17 protein.

In certain embodiments, the protein comprises: a) a fusion polypeptide comprising a first Fc polypeptide linked to an iduronate 2-sulfatase (IDS) amino acid sequence, wherein the fusion polypeptide comprises SEQ ID NO: 35, 36, 37, 67, 68 or 69; and b) a second Fc polypeptide comprising SEQ ID NO: 18 or 47. The protein comprising any combination of the aforementioned sequences in a) and b) is also referred to as ETV:IDS 35.21 protein.

In certain embodiments, a protein as described herein is comprised within a pharmaceutical composition. In certain embodiments, the pharmaceutical composition further comprises a pharmaceutically acceptable excipient. For example, the pharmaceutical composition can include at least one of (a) a buffer; and (b) an isotonicity agent, such as a salt.

In certain embodiments, the pharmaceutical composition further comprises one or more additional components such as, e.g., a surfactant; and/or one or more stabilizers. Exemplary pharmaceutical compositions containing proteins that comprise an IDS amino acid sequence and a TfR binding domain, such as ETV:IDS, are described in WO 2020/206320.

### Subjects

The term “subject,” “individual,” and “patient,” as used interchangeably herein, refers to a mammal, including but not limited to humans, non-human primates, rodents (e.g., rats, mice, and guinea pigs), rabbits, cows, pigs, horses, and other mammalian species. In certain embodiments described herein, the subject is a human subject.

In certain embodiments, the subject is a male subject.

In certain embodiments, the subject is a human male subject.

In certain embodiments, the subject is from about 1 month to 30 years of age. In certain embodiments, the subject is from about 6 months to 30 years of age. In certain embodiments, the subject is from about 1 to 30 years of age. In certain embodiments, the subject is from about 1 to 25 years of age. In certain embodiments, the subject is from about 1 to 18 years of age. In certain embodiments, the subject is from about 2 to 18 years of age. In certain embodiments, the subject is from about 2 to 15 years of age. In certain embodiments, the subject is from about 2 to 10 years of age. In certain embodiments, the subject is from about 5 to 10 years of age. In certain embodiments, the subject is less than 18 years of age. In certain embodiments, the subject is less than 4 years of age. In certain embodiments, the subject is less than 2 years of age. In certain embodiments, the subject is less than 1 year of age. In certain embodiments, the subject is more than 1 year of age. In certain embodiments, the subject is about 1, 2, 3, 4, 5, 6, 7, 8, 9, 10, 11, 12, 13, 14, 15, 16, 17 or 18 years of age.

In certain embodiments, the subject has a weight of ≥5 kg. In certain embodiments, the subject has a weight of ≥9 kg. In certain embodiments, the subject has a weight of ≥15 kg. In certain embodiments, the subject has a weight of ≥19 kg. In certain embodiments, the subject has a weight of ≥23 kg. In certain embodiments, the subject has a weight of ≥27 kg. In certain embodiments, the subject has a weight of ≥32 kg. In certain embodiments, the subject has a weight of ≥36 kg. In certain embodiments, the subject has a weight of ≥41 kg. In certain embodiments, the subject has a weight of ≥45 kg. In certain embodiments, the subject has a weight of ≥50 kg.

In certain embodiments, the subject has neuronopathic Hunter syndrome, also referred to as neuronopathic MPSII (nMPSII). In certain embodiments, the subject has non-neuronopathic Hunter syndrome. In certain embodiments, the subject has Hunter syndrome with an unknown neuronopathic phenotype.

In certain embodiments, the subject has a documented mutation in the IDS gene. In certain embodiments, the subject has been diagnosed as having reduced IDS enzyme activity. In certain embodiments, subject has been diagnosed with Hunter syndrome based on reduced IDS enzyme activity and a documented mutation in the IDS gene. In certain embodiments, the subject has a same genetic mutation in the IDS gene as a blood relative with confirmed neuronopathic Hunter Syndrome/nMPS II. In certain embodiments, the subject has neuronopathic Hunter syndrome/nMPS II or has a same genetic mutation in the IDS gene as a blood relative with confirmed neuronopathic Hunter Syndrome/nMPS II.

In certain embodiments, the subject had previously received idursulfase enzyme replacement therapy (e.g., for more than 4 months, more than 6 months, more than 1 year, more than 18 months, more than 2 years, or longer) and then switched to the administration of a pharmaceutical composition comprising a protein described herein (e.g., ETV:IDS protein). In certain embodiments, the switch occurs without a washout period (i.e., switched from weekly intravenous idursulfase administration to weekly intravenous administration of a pharmaceutical composition comprising a protein described herein (e.g., ETV:IDS) without treatment interruption). In certain embodiments, the subject had pre-existing ADAs against IDS prior to administration of the pharmaceutical composition. In certain embodiments, the subject's pre-existing titer of anti-drug antibodies against IDS is greater than 100, 150, 200, 300, 400, 500, 1,000, 5,000, 25,000, 50,000, 75,000, 100,000, 500,000, 1,000,000, 10,000,000 or more. In certain embodiments, the subject's pre-existing titer of anti-drug antibodies against IDS ranges from 189 to greater than 11 million. In certain embodiments, the subject's pre-existing titer of anti-drug antibodies against IDS is greater than 11 million.

In certain embodiments, the incidence of ADAs does not increase relative to pre-treatment levels in the subject after administration of the pharmaceutical composition. In certain embodiments, the incidence of ADAs increases by less than about 10%, 9%, 8%, 7%, 6%, 5%, 4%, 3%, 2%, or 1% relative to pre-treatment levels in the subject after administration of the pharmaceutical composition. In certain embodiments, the formation of new ADAs or increase in existing ADAs relative to pre-treatment levels in the subject does not significantly diminish the efficacy of treatment with the pharmaceutical composition.

In certain embodiments, the subject has a cognitive deficit.

In certain embodiments, the subject has a behavioral deficit.

In certain embodiments, the subject has a deficit in their physical abilities.

In certain embodiments, a deficit in the subject is determined by comparing a baseline level in the subject prior to treatment with the pharmaceutical to a healthy subject or a subject that does not have Hunter syndrome.

### Administration, Therapeutically Effective Doses, and Treatment Regimens

In certain embodiments, a pharmaceutical composition described herein may be administered to a subject at a therapeutically effective amount or dose, e.g., a safe and therapeutically effect amount or dose.

In certain embodiments, the dose is from about 3 mg/kg to about 30 mg/kg of protein (e.g., ETV:IDS protein).

In certain embodiments, the dose is about 3 mg/kg of protein. In certain embodiments, the dose is about 7.5 mg/kg of protein. In certain embodiments, the dose is about 15 mg/kg of protein. In certain embodiments, the dose is about 30 mg/kg of protein.

In certain embodiments, such a dose described herein is a therapeutically effective dose. In certain embodiments, such a dose described herein is a safe and therapeutically effective dose (e.g., as determined using an assessment described herein, such as stabilization or decrease in urine total GAG concentration in the subject).

In certain embodiments, the pharmaceutical composition is administered weekly. In certain embodiments, the pharmaceutical composition is administered weekly for at least about 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 weeks or more. In certain embodiments, the pharmaceutical composition is administered weekly for at least about 12 weeks. In certain embodiments, the pharmaceutical composition is administered weekly for at least about 23 weeks. In certain embodiments, the pharmaceutical composition is administered weekly for at least about 48 weeks.

Dosages may be varied according to several factors, including the chosen route of administration, the formulation of the composition, patient response, the severity of the condition, the subject's weight, the subject's age, and the judgment of the prescribing physician. The dosage can be increased or decreased over time, as required by an individual patient. In some embodiments, a patient initially is given a low dose, which is then increased to an efficacious dosage tolerable to the patient.

In one embodiment, the pharmaceutical composition is administered intraperitoneally.

In some embodiments, the pharmaceutical composition is administered intravenously. Intravenous administration can be by infusion, e.g., over a period of from about 10 to about 30 minutes, or over a period of at least 1 hour, 2 hours, 3 hours, 4 hours, 6 hours, 8 hours, or 10 hours. In some embodiments, the pharmaceutical composition is administered intravenously over a period of from about 20 minutes to 6 hours, or from about 30 minutes to 4 hours.

A pharmaceutical composition described herein (e.g., comprising ETV:IDS) may be administered to a subject as part of a treatment regimen. For example, in certain embodiments, the pharmaceutical composition is administered weekly. In certain embodiments, the pharmaceutical composition is intravenously administered at a particular dosage and rate, wherein the parameters may be modified over time to account for treatment efficacy and tolerability. In certain embodiments, an initial dose of the pharmaceutical composition is a therapeutically effective dose (e.g., a therapeutically effective dose described herein). In certain embodiments, subsequent doses of the pharmaceutical composition are reduced or increased.

For example, in certain embodiments a subject's treatment regimen may be modified to resolve an IRR. In some embodiments, the dosage of the pharmaceutical composition may be reduced to address an IRR. In some embodiments, infusion rate of the pharmaceutical composition may be reduced. In some embodiments, a reduction in the dosage may be implemented in combination with a reduced infusion rate of the pharmaceutical composition. In certain embodiments, additional subsequent dosages may be further lowered and/or the infusion rates may be further lowered to resolve an IRR. Over time, a subject that has experienced an IRR may be able to tolerate infusion of the pharmaceutical composition without experiencing an IRR. Thus, in certain embodiments, additional subsequent dosages may be increased and/or the infusion rates may be increased (e.g., increased to the initial dose and/or rate that was administered to the subject prior to the IRR).

In certain embodiments, methods of resolving an IRR may further comprise administering one or more therapeutic agents useful for treating an IRR to a subject (e.g., as part of a treatment regimen). Such therapeutic agents include, but are not limited to, an anti-histamine, an anti-pyretic, a corticosteroid, and combinations thereof. In certain embodiments, the one or more therapeutic agents comprise acetaminophen. In certain embodiments, the one or more therapeutic agents comprise diphenhydramine. In certain embodiments, the one or more therapeutic agents comprise epinephrine. In certain embodiments, the one or more therapeutic agents comprise a combination of acetaminophen and at least one antihistamine. In certain embodiments, the one or more therapeutic agents comprise a combination of acetaminophen and diphenhydramine. In certain embodiments, the one or more therapeutic agents comprise a combination of acetaminophen, diphenhydramine, and epinephrine.

When a combination of two or more therapeutic agents that are useful for treating an IRR are administered, they can be formulated separately or in a single composition. When the agents are formulated separately, they can be administered at essentially the same time, i.e., concurrently, or at separately staggered times, i.e., sequentially.

In certain embodiments, the one or more therapeutic agents are administered to the subject prior to the infusion of the pharmaceutical composition (e.g., prior to the subsequent dose of the pharmaceutical composition). In certain embodiments, the one or more therapeutic agents and the pharmaceutical composition are co-administered to the subject. For example, co-administration may involve interrupting the infusion of the pharmaceutical composition and administering one or more therapeutic agents that are useful for treating an IRR prior to continuing the infusion. In certain embodiments, the one or more therapeutic agents are administered to the subject after the administration of the pharmaceutical composition (e.g., after the administration of the subsequent dose of the pharmaceutical composition).

### CERTAIN EMBODIMENTS: SECTIONS A-E

### Embodiment Section A

Embodiment 1a. Certain embodiments provide a method of providing added peripheral iduronate 2-sulfatase (IDS) activity relative to a standard-of-care treatment to a subject with Hunter syndrome, comprising administering to the subject a therapeutically effective dose of a pharmaceutical composition comprising a protein, wherein the administration of the pharmaceutical composition reduces serum levels of keratan sulfate (KS) in the subject relative to a baseline level measured in the subject prior to the administration, and wherein the protein comprises:
a) a fusion polypeptide comprising a first Fc polypeptide linked to an iduronate 2-sulfatase (IDS) amino acid sequence, wherein the fusion polypeptide comprises SEQ ID NO:35, 36, 37, 67, 68 or 69; and
b) a second Fc polypeptide comprising SEQ ID NO:29 or 56.

Embodiment 2a. Certain embodiments provide a method of providing added peripheral iduronate 2-sulfatase (IDS) activity relative to a standard-of-care treatment to a subject with Hunter syndrome, comprising administering to the subject a therapeutically effective dose of a pharmaceutical composition comprising a protein, wherein the administration of the pharmaceutical composition reduces serum levels of keratan sulfate (KS) in the subject to a baseline level measured in a healthy subject or in a subject that does not have Hunter syndrome, and wherein the protein comprises:
a) a fusion polypeptide comprising a first Fc polypeptide linked to an iduronate 2-sulfatase (IDS) amino acid sequence, wherein the fusion polypeptide comprises SEQ ID NO:35, 36, 37, 67, 68 or 69; and
b) a second Fc polypeptide comprising SEQ ID NO:29 or 56.

Embodiment 3a. Certain embodiments provide a method of reducing and/or normalizing serum keratan sulfate (KS) levels in a subject with Hunter syndrome, comprising administering to the subject a therapeutically effective dose of a pharmaceutical composition comprising a protein, wherein the protein comprises:
a) a fusion polypeptide comprising a first Fc polypeptide linked to an iduronate 2-sulfatase (IDS) amino acid sequence, wherein the fusion polypeptide comprises SEQ ID NO:35, 36, 37, 67, 68 or 69; and
b) a second Fc polypeptide comprising SEQ ID NO:29 or 56.

Embodiment 4a. Certain embodiments provide a method for evaluating peripheral IDS activity in a subject having Hunter syndrome, comprising:
(a) determining a first marker profile by detecting the level of keratan sulfate (KS) in a sample from the subject obtained at baseline or prior to initiation of treatment with a pharmaceutical composition comprising a protein;
(b) administering to the subject the pharmaceutical composition comprising the protein;
(c) determining a second marker profile by detecting the level of KS in a sample from the subject obtained at a time point after administration of the pharmaceutical composition comprising the protein; and
(d) comparing the first and the second marker profiles;
wherein a decrease in KS levels between the first and the second marker profiles indicates added peripheral IDS activity in the subject, and
wherein the protein comprises: (i) a fusion polypeptide comprising a first Fc polypeptide linked to an iduronate 2-sulfatase (IDS) amino acid sequence, wherein the fusion polypeptide comprises SEQ ID NO:35, 36, 37, 67, 68 or 69; and (ii) a second Fc polypeptide comprising SEQ ID NO:29 or 56.

Embodiment 5a. The method of any one of embodiments 1a-4a, wherein the subject's serum heparan sulfate (HS) and/or dermatan sulfate (DS) levels decrease after administration of the pharmaceutical composition.

Embodiment 6a. The method of any one of embodiments 1a-5a, wherein the subject's urine heparan sulfate (HS) and/or dermatan sulfate (DS) levels decrease after administration of the pharmaceutical composition.

Embodiment 7a. The method of any one of embodiments 1a-6a, wherein the subject is a human subject.

Embodiment 8a. The method of embodiment 7a, wherein the subject is a human male subject.

Embodiment 9a. The method of any one of embodiments 1a-8a, wherein the subject has neuronopathic Hunter syndrome (nMPS II) or has a same genetic mutation in the IDS gene as a blood relative with confirmed nMPS II.

Embodiment 10a. The method of any one of embodiments 1a-9a, wherein the subject had previously received idursulfase enzyme replacement therapy and switched to the administration of the pharmaceutical composition.

Embodiment 11a. The method of embodiment 10a, wherein the subject was switched from administration of idursulfase enzyme replacement therapy to administration of the pharmaceutical composition without a washout period.

Embodiment 12a. The method of any one of embodiments 1a-11a, wherein the subject had pre-existing anti-drug antibodies against IDS prior to administration of the pharmaceutical composition.

Embodiment 13a. The method of embodiment 12a, wherein the subject's titer of anti-drug antibodies against IDS ranges from 189 to greater than 11 million.

Embodiment 14a. The method of embodiment 13a, wherein the subject's titer of anti-drug antibodies against IDS is greater than 11 million.

Embodiment 15a. The method of any one of embodiments 1a-14a, wherein the therapeutically effective dose is from about 3 mg/kg to about 30 mg/kg of protein.

Embodiment 16a. The method of embodiment 15a, wherein the therapeutically effective dose is about 3 mg/kg of protein.

Embodiment 17a. The method of embodiment 15a, wherein the therapeutically effective dose is about 7.5 mg/kg of protein.

Embodiment 18a. The method of embodiment 15a, wherein the therapeutically effective dose is about 15 mg/kg of protein.

Embodiment 19a. The method of embodiment 15a, wherein the therapeutically effective dose is about 30 mg/kg of protein.

Embodiment 20a. The method of any one of embodiments 1a-19a, wherein the pharmaceutical composition is administered weekly.

Embodiment 21a. The method of any one of embodiments 1a-20a, wherein the pharmaceutical composition is administered to the subject intravenously.

Embodiment 22a. The method of any one of embodiments 1a-21a, wherein the pharmaceutical composition further comprises a pharmaceutically acceptable excipient.

Embodiment 23a. The method of any one of embodiments 1a-22a, wherein the fusion polypeptide comprises SEQ ID NO: 35; and the second Fc polypeptide comprises SEQ ID NO: 29.

Embodiment 24a. The method of any one of embodiments 1a-22a, wherein the fusion polypeptide is SEQ ID NO: 35; and the second Fc polypeptide is SEQ ID NO: 29.

Embodiment 25a. The method of any one of embodiments 1a-22a, wherein the fusion polypeptide comprises SEQ ID NO: 67; and the second Fc polypeptide comprises SEQ ID NO: 56.

Embodiment 26a. The method of any one of embodiments 1a-22a, wherein the fusion polypeptide is SEQ ID NO: 67; and the second Fc polypeptide is SEQ ID NO: 56.

Embodiment 27a. The method of any one of embodiments 1a-22a, wherein the fusion polypeptide comprises SEQ ID NO: 37; and the second Fc polypeptide comprises SEQ ID NO: 29.

Embodiment 28a. The method of any one of embodiments 1a-22a, wherein the fusion polypeptide is SEQ ID NO: 37; and the second Fc polypeptide is SEQ ID NO: 29.

Embodiment 29a. The method of any one of embodiments 1a-22a, wherein the fusion polypeptide comprises SEQ ID NO: 69; and the second Fc polypeptide comprises SEQ ID NO: 56.

Embodiment 30a. The method of any one of embodiments 1a-22a, wherein the fusion polypeptide is SEQ ID NO: 69; and the second Fc polypeptide is SEQ ID NO: 56.

### Embodiment Section B

Embodiment 1b. A method of providing added peripheral iduronate 2-sulfatase (IDS) activity relative to a standard-of-care treatment to a subject with Hunter syndrome, comprising administering to the subject a therapeutically effective dose of a pharmaceutical composition comprising a protein, wherein the administration of the pharmaceutical composition reduces serum levels of keratan sulfate (KS) in the subject relative to a baseline level measured in the subject prior to the administration, and wherein the protein comprises an IDS amino acid sequence and a transferrin receptor (TfR) binding domain.

Embodiment 2b. A method of providing added peripheral iduronate 2-sulfatase (IDS) activity relative to a standard-of-care treatment to a subject with Hunter syndrome, comprising administering to the subject a therapeutically effective dose of a pharmaceutical composition comprising a protein, wherein the administration of the pharmaceutical composition reduces serum levels of keratan sulfate (KS) in the subject to a baseline level measured in a healthy subject or in a subject that does not have Hunter syndrome, and wherein the protein comprises an IDS amino acid sequence and a transferrin receptor (TfR) binding domain.

Embodiment 3b. A method of reducing and/or normalizing serum keratan sulfate (KS) levels in a subject with Hunter syndrome, comprising administering to the subject a therapeutically effective dose of a pharmaceutical composition comprising a protein, wherein the protein comprises an iduronate 2-sulfatase (IDS) amino acid sequence and a transferrin receptor (TfR) binding domain.

Embodiment 4b. A method for evaluating peripheral iduronate 2-sulfatase (IDS) activity in a subject having Hunter syndrome, comprising:
(a) determining a first marker profile by detecting the level of keratan sulfate (KS) in a sample from the subject obtained at baseline or prior to initiation of treatment with a pharmaceutical composition comprising a protein;
(b) administering to the subject the pharmaceutical composition comprising the protein;
(c) determining a second marker profile by detecting the level of KS in a sample from the subject obtained at a time point after administration of the pharmaceutical composition comprising the protein; and
(d) comparing the first and the second marker profiles;
wherein a decrease in KS levels between the first and the second marker profiles indicates added peripheral IDS activity in the subject, and
wherein the protein comprises an IDS amino acid sequence and a transferrin receptor (TfR) binding domain.

Embodiment 5b. The method of any one of embodiments 1b-4b, wherein the subject's serum heparan sulfate (HS) and/or dermatan sulfate (DS) levels decrease after administration of the pharmaceutical composition.

Embodiment 6b. The method of any one of embodiments 1b-5b, wherein the subject's urine heparan sulfate (HS) and/or dermatan sulfate (DS) levels decrease after administration of the pharmaceutical composition.

Embodiment 7b. The method of any one of embodiments 1b-6b, wherein the subject's total urine GAG levels decrease after administration of the pharmaceutical composition.

Embodiment 8b. The method of any one of embodiments 1b-7b, wherein the subject is a human subject.

Embodiment 9b. The method of embodiment 8b, wherein the subject is a human male subject.

Embodiment 10b. The method of any one of embodiments 1b-9b, wherein the subject has neuronopathic Hunter syndrome (nMPS II) or has a same genetic mutation in the IDS gene as a blood relative with confirmed nMPS II.

Embodiment 11b. The method of any one of embodiments 1b-10b, wherein the subject had previously received idursulfase enzyme replacement therapy and switched to the administration of the pharmaceutical composition.

Embodiment 12b. The method of embodiment 11c, wherein the subject was switched from administration of idursulfase enzyme replacement therapy to administration of the pharmaceutical composition without a washout period.

Embodiment 13b. The method of any one of embodiments 1b-12b, wherein the subject had pre-existing anti-drug antibodies against IDS prior to administration of the pharmaceutical composition.

Embodiment 14b. The method of embodiment 13b, wherein the subject's titer of anti-drug antibodies against IDS ranges from 189 to greater than 11 million.

Embodiment 15b. The method of embodiment 14b, wherein the subject's titer of anti-drug antibodies against IDS is greater than 11 million.

Embodiment 16b. The method of any one of embodiments 1b-15b, wherein the therapeutically effective dose is from about 3 mg/kg to about 30 mg/kg of protein.

Embodiment 17b. The method of embodiment 16b, wherein the therapeutically effective dose is about 3 mg/kg of protein.

Embodiment 18b. The method of embodiment 16b, wherein the therapeutically effective dose is about 7.5 mg/kg of protein.

Embodiment 19b. The method of embodiment 16b, wherein the therapeutically effective dose is about 15 mg/kg of protein.

Embodiment 20b. The method of embodiment 16b, wherein the therapeutically effective dose is about 30 mg/kg of protein.

Embodiment 21b. The method of any one of embodiments 1b-20b, wherein the pharmaceutical composition is administered weekly.

Embodiment 22b. The method of embodiment 21b, wherein the reduction and/or normalization of KS is sustained after at least 12 weekly doses.

Embodiment 23b. The method of embodiment 21b, wherein the reduction and/or normalization of KS is sustained after at least 23 weekly doses.

Embodiment 24b. The method of any one of embodiments 21b-23b, wherein the subject's serum heparan sulfate (HS) and/or dermatan sulfate (DS) levels decrease and/or normalize relative to a baseline level by at least 12 weekly doses of the pharmaceutical composition.

Embodiment 25b. The method of any one of embodiments 21b-23b, wherein the subject's serum heparan sulfate (HS) and/or dermatan sulfate (DS) levels decrease and/or normalize relative to a baseline level by at least 23 weekly doses of the pharmaceutical composition.

Embodiment 26b. The method of any one of embodiments 21b-23b, wherein the subject's serum heparan sulfate (HS) and/or dermatan sulfate (DS) levels decrease and/or normalize relative to a baseline level by at least 48 weekly doses of the pharmaceutical composition.

Embodiment 27b. The method of any one of embodiments 21b-26b, wherein the subject's urine heparan sulfate (HS) and/or dermatan sulfate (DS) levels decrease and/or normalize relative to a baseline level after by at least 12 weekly doses of the pharmaceutical composition.

Embodiment 28b. The method of any one of embodiments 21b-26b, wherein the subject's urine heparan sulfate (HS) and/or dermatan sulfate (DS) levels decrease and/or normalize relative to a baseline level after by at least 23 weekly doses of the pharmaceutical composition.

Embodiment 29b. The method of any one of embodiments 21b-26b, wherein the subject's urine heparan sulfate (HS) and/or dermatan sulfate (DS) levels decrease and/or normalize relative to a baseline level after by at least 48 weekly doses of the pharmaceutical composition.

Embodiment 30b. The method of any one of embodiments 21b-26b, wherein the subject's total urine GAG levels decrease relative to a baseline level after at least 12 weekly doses of the pharmaceutical composition.

Embodiment 31b. The method of any one of embodiments 21b-26b, wherein the subject's total urine GAG levels decrease relative to a baseline level after at least 23 weekly doses of the pharmaceutical composition.

Embodiment 32b. The method of any one of embodiments 21b-26b, wherein the subject's total urine GAG levels decrease relative to a baseline level after at least 48 weekly doses of the pharmaceutical composition.

Embodiment 33b. The method of any one of embodiments 1b-32b, wherein the pharmaceutical composition is administered to the subject intravenously.

Embodiment 34b. The method of any one of embodiments 1b-33b, wherein the pharmaceutical composition further comprises a pharmaceutically acceptable excipient.

Embodiment 35b. A method of providing added peripheral iduronate 2-sulfatase (IDS) activity relative to a standard-of-care treatment to a subject with Hunter syndrome, comprising administering to the subject a therapeutically effective dose of a pharmaceutical composition comprising a protein, wherein the administration of the pharmaceutical composition normalizes levels of urine heparan sulfate (HS) and/or dermatan sulfate (DS) in the subject by at least 12 weekly doses, and wherein normalization is sustained after at least 23 weekly doses, and wherein the protein comprises an IDS amino acid sequence and a transferrin receptor (TfR) binding domain.

Embodiment 36b. A method of providing added peripheral iduronate 2-sulfatase (IDS) activity relative to a standard-of-care treatment to a subject with Hunter syndrome, comprising administering to the subject at least 23 weekly therapeutically effective doses of a pharmaceutical composition comprising a protein, wherein the administration of the pharmaceutical composition normalizes levels of urine heparan sulfate (HS) and/or dermatan sulfate (DS) in the subject by at least 12 weekly doses, and wherein normalization is sustained after at least 23 weekly doses, and wherein the protein comprises an IDS amino acid sequence linked to an Fc polypeptide; and a transferrin receptor (TfR) binding domain.

Embodiment 37b. A method of normalizing urine heparan sulfate (HS) and/or dermatan sulfate (DS) levels in a subject with Hunter syndrome, comprising administering to the subject a therapeutically effective dose of a pharmaceutical composition comprising a protein, wherein the HS and/or DS levels in the subject are normalized by at least 12 weekly doses, wherein normalization is sustained after at least 23 weekly doses, and wherein the protein comprises an IDS amino acid sequence and a transferrin receptor (TfR) binding domain.

Embodiment 38b. A method of normalizing urine heparan sulfate (HS) and/or dermatan sulfate (DS) levels in a subject with Hunter syndrome, comprising administering to the subject at least 23 weekly therapeutically effective doses of a pharmaceutical composition comprising a protein, wherein the HS and/or DS levels in the subject are normalized by at least 12 weekly doses, wherein normalization is sustained after at least 23 weekly doses, and wherein the protein comprises an IDS amino acid sequence and a transferrin receptor (TfR) binding domain.

Embodiment 39b. The method of any one of embodiments 35b-38b, wherein the normalization of HS and/or DS is sustained after at least 30 weekly doses.

Embodiment 40b. The method of any one of embodiments 35b-38b, wherein the normalization of HS and/or DS is sustained after at least 36 weekly doses.

Embodiment 41b. The method of any one of embodiments 35b-38b, wherein the normalization of HS and/or DS is sustained after at least 42 weekly doses.

Embodiment 42b. The method of any one of embodiments 35b-38b, wherein the normalization of HS and/or DS is sustained after at least 48 weekly doses.

Embodiment 43b. The method of any one of embodiments 35b-38b, wherein the subject's total urine GAG levels decrease relative to a baseline level measured prior to administration after at least 12 weekly doses.

Embodiment 44b. The method of any one of embodiments 35b-38b, wherein the subject's total urine GAG levels decrease relative to a baseline level measured prior to administration after at least 23 weekly doses.

Embodiment 45b. The method of any one of embodiments 35b-38b, wherein the subject's total urine GAG levels decrease relative to a baseline level measured prior to administration after at least 48 weekly doses.

Embodiment 46b. The method of any one of embodiments 35b-45b, wherein the subject is a human subject.

Embodiment 47b. The method of any one of embodiments 35b-46b, wherein the subject had previously received idursulfase enzyme replacement therapy and switched to the administration of the pharmaceutical composition.

Embodiment 48b. The method of any one of embodiments 35b-47b, wherein the therapeutically effective dose is from about 3 mg/kg to about 30 mg/kg of protein.

Embodiment 49b. The method of embodiment 48b, wherein the therapeutically effective dose is about 3 mg/kg of protein.

Embodiment 50b. The method of embodiment 48b, wherein the therapeutically effective dose is about 7.5 mg/kg of protein.

Embodiment 51b. The method of embodiment 48b, wherein the therapeutically effective dose is about 15 mg/kg of protein.

Embodiment 52b. The method of embodiment 48b, wherein the therapeutically effective dose is about 30 mg/kg of protein.

Embodiment 53b. The method of any one of embodiments 35b-52b, wherein the pharmaceutical composition is administered to the subject intravenously.

Embodiment 54b. The method of any one of embodiments 1b-53b, wherein the protein comprises an IDS amino acid sequence linked to an Fc polypeptide; and a transferrin receptor (TfR) binding domain.

Embodiment 55b. The method of embodiment 54b, wherein the protein comprises a fusion polypeptide comprising a first Fc polypeptide linked to the IDS amino acid sequence; a second Fc polypeptide that forms an Fc dimer with the first Fc polypeptide; and a transferrin receptor (TfR) binding domain.

Embodiment 56b. The method of any one of embodiments 1b-55b, wherein the TfR binding domain is comprised within an Fc polypeptide.

Embodiment 57b. The method of any one of embodiments 1b-56b, wherein the protein does not comprise an immunoglobulin heavy and/or light chain variable region sequence or an antigen-binding portion thereof.

Embodiment 58b. The method of any one of embodiments 1b-57b, wherein the protein comprises:
(a) a fusion polypeptide comprising a first Fc polypeptide linked to an iduronate 2-sulfatase (IDS) amino acid sequence, wherein the fusion polypeptide comprises SEQ ID NO: 35, 36, 37, 67, 68 or 69; and
(b) a second Fc polypeptide comprising SEQ ID NO: 29 or 56.

Embodiment 59b. The method of embodiment 58b, wherein the fusion polypeptide comprises SEQ ID NO: 35; and the second Fc polypeptide comprises SEQ ID NO: 29.

Embodiment 60b. The method of embodiment 58b, wherein the fusion polypeptide is SEQ ID NO: 35; and the second Fc polypeptide is SEQ ID NO: 29.

Embodiment 61b. The method of embodiment 58b, wherein the fusion polypeptide comprises SEQ ID NO: 67; and the second Fc polypeptide comprises SEQ ID NO: 56.

Embodiment 62b. The method of embodiment 58b, wherein the fusion polypeptide is SEQ ID NO: 67; and the second Fc polypeptide is SEQ ID NO: 56.

Embodiment 63b. The method of embodiment 58b, wherein the fusion polypeptide comprises SEQ ID NO: 37; and the second Fc polypeptide comprises SEQ ID NO: 29.

Embodiment 64b. The method of embodiment 58b, wherein the fusion polypeptide is SEQ ID NO: 37; and the second Fc polypeptide is SEQ ID NO: 29.

Embodiment 65b. The method of embodiment 58b, wherein the fusion polypeptide comprises SEQ ID NO: 69; and the second Fc polypeptide comprises SEQ ID NO: 56.

Embodiment 66b. The method of embodiment 58b, wherein the fusion polypeptide is SEQ ID NO: 69; and the second Fc polypeptide is SEQ ID NO: 56.

### Embodiment Section C

Embodiment 1c. A method of providing a clinical benefit to a subject with Hunter syndrome, comprising administering to the subject a therapeutically effective dose of a pharmaceutical composition comprising a protein, wherein the administration of the pharmaceutical composition produces an improvement in a clinical outcome as measured by Clinician Global Impression of Change (CGI-C) and/or Parent/Caregiver Global Impression of Change (PGI-C) relative to baseline levels measured in the subject prior to the administration, and wherein the protein comprises:
a) a fusion polypeptide comprising a first Fc polypeptide linked to an iduronate 2-sulfatase (IDS) amino acid sequence, wherein the fusion polypeptide comprises SEQ ID NO:35, 36, 37, 67, 68, or 69; and
b) a second Fc polypeptide comprising SEQ ID NO:29 or 56.

Embodiment 2c. The method of embodiment 1c, wherein the administration of the pharmaceutical composition produces an improvement in the subject's overall Hunter syndrome symptoms, as measured by the CGI-C and/or PGI-C.

Embodiment 3c. The method of embodiment 1c or 2c, wherein the administration of the pharmaceutical composition produces an improvement in the subject's cognitive abilities, as measured by the CGI-C and/or PGI-C.

Embodiment 4c. The method of any one of embodiments 1c-3c wherein the administration of the pharmaceutical composition produces an improvement in the subject's behavior, as measured by the CGI-C and/or PGI-C.

Embodiment 5c. The method of any one of embodiments 1c-4c, wherein the administration of the pharmaceutical composition produces an improvement in the subject's physical abilities, as measured by the CGI-C and/or PGI-C.

Embodiment 6c. The method of any one of embodiments 1c-5c, wherein the clinical outcome is minimally improved, as measured by the CGI-C and/or PGI-C.

Embodiment 7c. The method of any one of embodiments 1c-5c, wherein the clinical outcome is much improved, as measured by the CGI-C and/or PGI-C.

Embodiment 8c. The method of any one of embodiments 1c-5c, wherein the clinical outcome is very much improved, as measured by the CGI-C and/or PGI-C.

Embodiment 9c. The method of any one of embodiments 1c-8c, wherein the improvement is measured by the CGI-C.

Embodiment 10c. The method of any one of embodiments 1c-8c, wherein the improvement is measured by the PGI-C.

Embodiment 1c. The method of any one of embodiments 1c-10c, wherein the subject is a human subject.

Embodiment 12c. The method of embodiment 11c, wherein the subject is a human male subject.

Embodiment 13c. The method of any one of embodiments 1c-12c, wherein the subject has neuronopathic Hunter syndrome (nMPS II) or has a same genetic mutation in the IDS gene as a blood relative with confirmed nMPS II.

Embodiment 14c. The method of any one of embodiments 1c-13c, wherein the subject had previously received idursulfase enzyme replacement therapy and switched to the administration of the pharmaceutical composition.

Embodiment 15c. The method of embodiment 14c, wherein the subject was switched from administration of idursulfase enzyme replacement therapy to administration of the pharmaceutical composition without a washout period.

Embodiment 16c. The method of any one of embodiments 1c-15c, wherein the subject had pre-existing anti-drug antibodies against IDS prior to administration of the pharmaceutical composition.

Embodiment 17c. The method of embodiment 16c, wherein the subject's titer of anti-drug antibodies against IDS ranges from 189 to greater than 11 million.

Embodiment 18c. The method of embodiment 17c, wherein the subject's titer of anti-drug antibodies against IDS is greater than 11 million.

Embodiment 19c. The method of any one of embodiments 1c-18c, wherein the therapeutically effective dose is from about 3 mg/kg to about 30 mg/kg of protein.

Embodiment 20c. The method of embodiment 19c, wherein the therapeutically effective dose is about 3 mg/kg of protein.

Embodiment 21c. The method of embodiment 19c, wherein the therapeutically effective dose is about 7.5 mg/kg of protein.

Embodiment 22c. The method of embodiment 19c, wherein the therapeutically effective dose is about 15 mg/kg of protein.

Embodiment 23c. The method of embodiment 19c, wherein the therapeutically effective dose is about 30 mg/kg of protein.

Embodiment 24c. The method of any one of embodiments 1c-23c, wherein the pharmaceutical composition is administered weekly.

Embodiment 25c. The method of embodiment 24c, wherein the pharmaceutical composition is administered weekly for at least 12 weeks.

Embodiment 26c. The method of embodiment 24c, wherein the pharmaceutical composition is administered weekly for at least 23 weeks.

Embodiment 27c. The method of any one of embodiments 1c-26c, wherein the pharmaceutical composition is administered to the subject intravenously.

Embodiment 28c. The method of any one of embodiments 1c-27c, wherein the pharmaceutical composition further comprises a pharmaceutically acceptable excipient.

Embodiment 29c. The method of any one of embodiments 1c-28c, wherein the fusion polypeptide comprises SEQ ID NO: 35; and the second Fc polypeptide comprises SEQ ID NO: 29.

Embodiment 30c. The method of any one of embodiments 1c-28c, wherein the fusion polypeptide is SEQ ID NO: 35; and the second Fc polypeptide is SEQ ID NO: 29.

Embodiment 31c. The method of any one of embodiments 1c-28c, wherein the fusion polypeptide comprises SEQ ID NO: 67; and the second Fc polypeptide comprises SEQ ID NO: 56.

Embodiment 32c. The method of any one of embodiments 1c-28c, wherein the fusion polypeptide is SEQ ID NO: 67; and the second Fc polypeptide is SEQ ID NO: 56.

Embodiment 33c. The method of any one of embodiments 1c-28c, wherein the fusion polypeptide comprises SEQ ID NO: 37; and the second Fc polypeptide comprises SEQ ID NO: 29.

Embodiment 34c. The method of any one of embodiments 1c-28c, wherein the fusion polypeptide is SEQ ID NO: 37; and the second Fc polypeptide is SEQ ID NO: 29.

Embodiment 35c. The method of any one of embodiments 1c-28c, wherein the fusion polypeptide comprises SEQ ID NO: 69; and the second Fc polypeptide comprises SEQ ID NO: 56.

Embodiment 36c. The method of any one of embodiments 1c-28c, wherein the fusion polypeptide is SEQ ID NO: 69; and the second Fc polypeptide is SEQ ID NO: 56.

### Embodiment Section D

Embodiment 1d. A method of improving treatment in a patient having non-neuronopathic Hunter syndrome, comprising switching the patient from idursulfase enzyme replacement therapy to treatment with a pharmaceutical composition comprising ETV:IDS protein, wherein the ETV:IDS protein comprises:
a) a fusion polypeptide comprising a first Fc polypeptide linked to an iduronate 2-sulfatase (IDS) amino acid sequence, wherein the fusion polypeptide comprises SEQ ID NO:35, 36, 37, 67, 68, or 69; and
b) a second Fc polypeptide comprising SEQ ID NO:29 or 56.

Embodiment 2d. The method of embodiment 1d, wherein switching the patient from idursulfase enzyme replacement therapy to treatment with the pharmaceutical composition comprising ETV:IDS reduces the level of a glycosaminoglycan (GAG) in the cerebrospinal fluid (CSF) of the patient relative to a baseline level in the patient measured before the switching.

Embodiment 3d. The method of embodiment 2d, wherein the level of the GAG in the CSF of the patient is reduced to a level measured in a healthy subject or a subject that does not have Hunter Syndrome.

Embodiment 4d. The method of embodiment 2d or 3d, wherein the GAG is heparan sulfate.

Embodiment 5d. The method of embodiment 2d or 3d, wherein the GAG is dermatan sulfate.

Embodiment 6d. The method of any one of embodiments 1d-5d, wherein switching the patient from idursulfase enzyme replacement therapy to treatment with the pharmaceutical composition comprising ETV:IDS reduces the total level of glycosaminoglycans (GAGs) in the urine of the patient relative to a baseline level measured in the patient before the switching, thereby providing added peripheral activity.

Embodiment 7d. The method of any one of embodiments 1d-6d, wherein the patient had pre-existing anti-drug antibodies against IDS prior to administration of the pharmaceutical composition.

Embodiment 8d. The method of any one of embodiments 1d-7d, wherein the treatment with the pharmaceutical composition comprising ETV:IDS comprises administering a dose of from about 3 mg/kg to about 30 mg/kg of ETV:IDS.

Embodiment 9d. The method of embodiment 8d, wherein the dose is about 3 mg/kg of ETV:IDS.

Embodiment 10d. The method of embodiment 8d, wherein the dose is about 7.5 mg/kg of ETV:IDS.

Embodiment 11d. The method of embodiment 8d, wherein the dose is about 15 mg/kg of ETV:IDS.

Embodiment 12d. The method of embodiment 8d, wherein the dose is about 30 mg/kg of ETV:IDS.

Embodiment 13d. The method of any one of embodiments 1d-12d, wherein the pharmaceutical composition is administered weekly.

Embodiment 14d. The method of embodiment 13d, wherein the pharmaceutical composition is administered weekly for at least 12 weeks.

Embodiment 15d. The method of embodiment 13d, wherein the pharmaceutical composition is administered weekly for at least 23 weeks.

Embodiment 16d. The method of any one of embodiments 1d-15d, wherein the pharmaceutical composition is administered intravenously.

Embodiment 17d. The method of any one of embodiments 1d-16d, wherein the pharmaceutical composition further comprises a pharmaceutically acceptable excipient.

Embodiment 18d. The method of any one of embodiments 1d-17d, wherein the fusion polypeptide comprises SEQ ID NO: 35; and the second Fc polypeptide comprises SEQ ID NO: 29.

Embodiment 19d. The method of any one of embodiments 1d-17d, wherein the fusion polypeptide is SEQ ID NO: 35; and the second Fc polypeptide is SEQ ID NO: 29.

Embodiment 20d. The method of any one of embodiments 1d-17d, wherein the fusion polypeptide comprises SEQ ID NO: 67; and the second Fc polypeptide comprises SEQ ID NO: 56.

Embodiment 21d. The method of any one of embodiments 1d-17d, wherein the fusion polypeptide is SEQ ID NO: 67; and the second Fc polypeptide is SEQ ID NO: 56.

Embodiment 22d. The method of any one of embodiments 1d-17d, wherein the fusion polypeptide comprises SEQ ID NO: 37; and the second Fc polypeptide comprises SEQ ID NO: 29.

Embodiment 23d. The method of any one of embodiments 1d-17d, wherein the fusion polypeptide is SEQ ID NO: 37; and the second Fc polypeptide is SEQ ID NO: 29.

Embodiment 24d. The method of any one of embodiments 1d-17d, wherein the fusion polypeptide comprises SEQ ID NO: 69; and the second Fc polypeptide comprises SEQ ID NO: 56.

Embodiment 25d. The method of any one of embodiments 1d-17d, wherein the fusion polypeptide is SEQ ID NO: 69; and the second Fc polypeptide is SEQ ID NO: 56.

### Embodiment Section E

Embodiment 1e. A method of resolving an infusion-related reaction (IRR) in a subject receiving treatment for Hunter syndrome, wherein the subject is being intravenously administered or was intravenously administered an initial dose of a pharmaceutical composition comprising a protein, the method comprising reducing the amount of a subsequent dose of the pharmaceutical composition and reducing the infusion rate of administration for the subsequent dose of the pharmaceutical composition, wherein the protein comprises:
a) a fusion polypeptide comprising a first Fc polypeptide linked to an iduronate 2-sulfatase (IDS) amino acid sequence, wherein the fusion polypeptide comprises SEQ ID NO:35, 36, 37, 67, 68, or 69; and
b) a second Fc polypeptide comprising SEQ ID NO:29 or 56.

Embodiment 2e. A method of resolving an IRR in a subject receiving treatment for Hunter syndrome, comprising modifying the subject's treatment regimen, wherein the treatment regimen comprises intravenous administration of an initial dose of a pharmaceutical composition comprising a protein, and the modified regimen comprises administering a reduced subsequent dose of the pharmaceutical composition to the subject at a reduced rate of infusion, and wherein the protein comprises:
a) a fusion polypeptide comprising a first Fc polypeptide linked to an iduronate 2-sulfatase (IDS) amino acid sequence, wherein the fusion polypeptide comprises SEQ ID NO:35, 36, 37, 67, 68, or 69; and
b) a second Fc polypeptide comprising SEQ ID NO:29 or 56.

Embodiment 3e. The method of embodiment 1e or 2e, wherein the IRR is anaphylaxis.

Embodiment 4e. The method of embodiment 3e, wherein the anaphylaxis meets the Sampson criteria.

Embodiment 5e. The method of any one of embodiments 1e-4e, wherein the method further comprises administering to the subject one or more therapeutic agents useful for treating an TRR, wherein the one or more agents are administered prior to the administration of the subsequent reduced dose, co-administered with the subsequent reduced dose, or are administered after the subsequent reduced dose.

Embodiment 6e. The method of embodiment 5e, wherein the one or more therapeutic agents are administered prior to the administration of the subsequent reduced dose.

Embodiment 7e. The method of embodiment 6e, wherein the one or more therapeutic agents are selected from the group consisting of: an anti-histamine, an anti-pyretic, a corticosteroid, and combinations thereof.

Embodiment 8e. The method of embodiment 7e, wherein the one or more therapeutic agents comprise a combination of acetaminophen and diphenhydramine.

Embodiment 9e. The method of any one of embodiments 1e-8e, wherein the subject is a human subject.

Embodiment 10e. The method of embodiment 9e, wherein the subject is a human male subject.

Embodiment 11e. The method of any one of embodiments 1e-10e, wherein the subject has neuronopathic Hunter syndrome (nMPS II) or has a same genetic mutation in the IDS gene as a blood relative with confirmed nMPS II.

Embodiment 12e. The method of any one of embodiments 1e-11e, wherein the subject had previously received idursulfase enzyme replacement therapy and switched to the administration of the pharmaceutical composition.

Embodiment 13e. The method of any one of embodiments 1e-12e, wherein the subject had pre-existing anti-drug antibodies against IDS prior to administration of the pharmaceutical composition.

Embodiment 14e. The method of embodiment 13e, wherein the subject's titer of anti-drug antibodies against IDS ranges from 189 to greater than 11 million.

Embodiment 15e. The method of embodiment 14a, wherein the subject's titer of anti-drug antibodies against IDS is greater than 11 million.

Embodiment 16e. The method of any one of embodiments 1e-15e, wherein the initial dose is from about 3 mg/kg to about 30 mg/kg of protein.

Embodiment 17e. The method of embodiment 16e, wherein the initial dose is about 3 mg/kg of protein.

Embodiment 18e. The method of embodiment 16e, wherein the initial dose is about 7.5 mg/kg of protein.

Embodiment 19e. The method of embodiment 16e, wherein the initial dose is about 15 mg/kg of protein.

Embodiment 20e. The method of embodiment 16e, wherein the initial dose is about 30 mg/kg of protein.

Embodiment 21e. The method of any one of embodiments 1e-20e, wherein the pharmaceutical composition is administered weekly.

Embodiment 22e. The method of any one of embodiments 1e-21e, wherein the pharmaceutical composition further comprises a pharmaceutically acceptable excipient.

Embodiment 23e. The method of any one of embodiments 1e-22e, wherein the fusion polypeptide comprises SEQ ID NO: 35; and the second Fc polypeptide comprises SEQ ID NO: 29.

Embodiment 24e. The method of any one of embodiments 1e-22e, wherein the fusion polypeptide is SEQ ID NO: 35; and the second Fc polypeptide is SEQ ID NO: 29.

Embodiment 25e. The method of any one of embodiments 1e-20e, wherein the fusion polypeptide comprises SEQ ID NO: 67; and the second Fc polypeptide comprises SEQ ID NO: 56.

Embodiment 26e. The method of any one of embodiments 1e-22e, wherein the fusion polypeptide is SEQ ID NO: 67; and the second Fc polypeptide is SEQ ID NO: 56.

Embodiment 27e. The method of any one of embodiments 1e-22e, wherein the fusion polypeptide comprises SEQ ID NO: 37; and the second Fc polypeptide comprises SEQ ID NO: 29.

Embodiment 28e. The method of any one of embodiments 1e-22e, wherein the fusion polypeptide is SEQ ID NO: 37; and the second Fc polypeptide is SEQ ID NO: 29.

Embodiment 29e. The method of any one of embodiments 1e-22e, wherein the fusion polypeptide comprises SEQ ID NO: 69; and the second Fc polypeptide comprises SEQ ID NO: 56.

Embodiment 30e. The method of any one of embodiments 1e-22e, wherein the fusion polypeptide is SEQ ID NO: 69; and the second Fc polypeptide is SEQ ID NO: 56.

### Certain Definitions

The term “subject,” “individual,” and “patient,” as used interchangeably herein, refer to a mammal, including but not limited to humans, non-human primates, rodents (e.g., rats, mice, and guinea pigs), rabbits, cows, pigs, horses, and other mammalian species. In one embodiment, the patient/subject is a human.

The term “subject in need thereof” refers to subject having MPS II (Hunter syndrome) that is in need of being treated with a protein as described herein (e.g., is in need of added peripheral benefit). For example, the subject may have elevated levels of at least one specified analyte (e.g., KS, HS and/or DS) compared to a subject that is healthy or does not have MPS II.

As used herein, the phrase “sample” or “physiological sample” is meant to refer to a biological sample obtained from a subject that contains an analyte of interest (e.g., a GAG or a ganglioside). In certain embodiments, the physiological sample comprises, e.g., CSF, urine, blood, serum, or plasma. In certain embodiments, the sample comprises CSF. In certain embodiments, the sample comprises urine. In certain embodiments, the sample comprises blood, plasma, or serum. In certain embodiments, the sample comprises serum.

The terms “obtaining a sample from a patient”, “obtained from a patient” and similar phrasing, is used to refer to obtaining the sample directly from the patient, as well as obtaining the sample indirectly from the patient through an intermediary individual (e.g., obtaining the sample from a courier who obtained the sample from a nurse who obtained the sample from the patient).

As described herein, levels of an analyte of interest (e.g., a GAG or ganglioside) may be compared to control or baseline levels. Such a control or baseline level may vary. For example, in certain embodiments, the term “control” may refer to a healthy subject or a subject that does not have Hunter syndrome (or a sample therefrom). Alternatively, the term “control” may refer to a Hunter syndrome patient that was not administered the pharmaceutical composition or to the subject prior to treatment (or a sample therefrom). Similarly, a “baseline level” may refer to a level or a range of levels that is measured in, e.g., a healthy individual or in a subject that does not have Hunter syndrome. In certain other embodiments described herein, a “baseline level” may also refer to a level or a range of levels in a Hunter syndrome patient that was not administered the pharmaceutical composition or in the subject prior to administration of the pharmaceutical composition.

In certain embodiments, a control value or baseline level may be established using data from a population of control subjects. In some embodiments, the population of subjects is matched to a test subject according to one or more patient characteristics such as age, sex, ethnicity, or other criteria. In some embodiments, the control value is established using the same type of sample from the population of subjects (e.g., a sample comprising serum) as is used for assessing the levels in the test subject.

As used herein, the term “normalized” refers to an analyte of interest having a level that falls within a normal range for the specified analyte, as determined by one or more healthy subjects or subjects that do not have Hunter syndrome. In some embodiments, a normalized level of an analyte is within the 10^{th} to 90^{th} percentile range from the determined normal range for the analyte.

The term “pharmaceutically acceptable excipient” refers to a non-active pharmaceutical ingredient that is biologically or pharmacologically compatible for use in humans or animals, such as but not limited to a buffer, carrier, or preservative.

The term “administer” refers to a method of delivering agents (e.g., an ETV:IDS therapy described herein or an agent useful for treating an IRR), compounds, or compositions (e.g., pharmaceutical composition) to the desired site of biological action. These methods include, but are not limited to, oral, topical delivery, parenteral delivery, intravenous delivery, intradermal delivery, intramuscular delivery, or intraperitoneal delivery. In one embodiment, the pharmaceutical composition comprising ETV:IDS protein described herein is administered intravenously. In one embodiment, the pharmaceutical composition comprising ETV:IDS protein described herein is administered intraperitoneally.

As used herein, “treatment” (and grammatical variations thereof such as “treat” or “treating”) refers to clinical intervention to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis.

As used herein, the terms “resolving an IRR” refers to preventing occurrence or recurrence of the IRR; and/or attenuation, amelioration or elimination of symptoms associated with the IRR.

The phrase “effective amount” means an amount of a compound described herein that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

A “therapeutically effective amount” of a substance/molecule disclosed herein may vary according to factors such as the disease state, age, sex, and weight of the individual, and the ability of the substance/molecule, to elicit a desired response in the individual. A therapeutically effective amount encompasses an amount in which any toxic or detrimental effects of the substance/molecule are outweighed by the therapeutically beneficial effects.

As used herein, the term “standard-of-care” refers to a therapy approved for the treatment of Hunter syndrome, including idursulfase enzyme replacement therapy (e.g., Elaprase).

An “iduronate sulfatase,” “iduronate-2-sulfatase,” or “IDS” as used herein refers to iduronate 2-sulfatase (EC 3.1.6.13), which is an enzyme involved in the lysosomal degradation of the glycosaminoglycans heparan sulfate and dermatan sulfate. Deficiency of IDS is associated with Mucopolysaccharidosis II, also known as Hunter syndrome. The term “IDS” or “IDS enzyme” as used herein, optionally as a component of a protein that comprises an Fc polypeptide, is catalytically active and encompasses wild-type IDS and functional variants, including allelic and splice variants, and catalytically active fragments thereof. In certain embodiments, the term “IDS” is used herein as a component of a protein that comprises an Fc polypeptide and is a catalytically active wild-type IDS or a fragment thereof. The sequence of human IDS isoform I, which is the human sequence designated as the canonical sequence, is available under UniProt entry P22304 and is encoded by the human IDS gene at Xq28. A “mature” IDS sequence as used herein refers to a form of a polypeptide chain that lacks the signal and propeptide sequences of the naturally occurring full-length polypeptide chain. The amino acid sequence of a mature human IDS polypeptide corresponds to amino acids 34-550 of the full-length human sequence. A “truncated” IDS sequence as used herein refers to a catalytically active fragment of the naturally occurring full-length polypeptide chain. The amino acid sequence of an exemplary truncated human IDS polypeptide corresponds to amino acids 26-550 of the full-length human sequence (see, underlined sequence shown in SEQ ID NO:36). The structure of human IDS has been well-characterized. An illustrative structure is available under PDB accession code 5FQL. The structure is also described in Nat. Comm. 8:15786 doi: 10.1038/ncomms15786, 2017. A catalytically active IDS fragment has at least 50%, at least 55%, at least 60%, at least 65%, at least 70%, at least 75%, at least 80%, at least 85%, at least 90%, or at least 95% of the activity of the corresponding full-length IDS or variant thereof, e.g., when assayed under identical conditions.

The term “peripheral IDS activity” or “peripheral activity” as used herein refers to the level of IDS enzymatic activity in the periphery of a subject.

A “transferrin receptor” or “TfR” as used herein refers to transferrin receptor protein 1. The human transferrin receptor 1 polypeptide sequence is set forth in SEQ ID NO:38. The term “transferrin receptor” also encompasses allelic variants of exemplary reference sequences, e.g., human sequences, that are encoded by a gene at a transferrin receptor protein 1 chromosomal locus. Full-length transferrin receptor protein includes a short N-terminal intracellular region, a transmembrane region, and a large extracellular domain. The extracellular domain is characterized by three domains: a protease-like domain, a helical domain, and an apical domain.

A “fusion protein” or “[IDS enzyme]-Fc fusion protein” as used herein refers to a dimeric protein comprising a first Fc polypeptide that is linked (e.g., fused) to an IDS enzyme, or a catalytically active fragment thereof (i.e., an “[IDS]-Fc fusion polypeptide”); and a second Fc polypeptide that forms an Fc dimer with the first Fc polypeptide. In certain embodiments, the second Fc polypeptide comprises modifications that confer binding to a transferrin receptor.

A “fusion polypeptide” or “[IDS enzyme]-Fc fusion polypeptide” as used herein refers to an Fc polypeptide that is linked (e.g., fused) to an IDS enzyme, or a catalytically active fragment thereof. The Fc polypeptide is linked to the IDS enzyme, or catalytically active fragment thereof, by a polypeptide linker.

As used herein, the term “Fc polypeptide” refers to the C-terminal region of a naturally occurring immunoglobulin heavy chain polypeptide that is characterized by an Ig fold as a structural domain. An Fc polypeptide contains constant region sequences including at least the CH2 domain and/or the CH3 domain and may contain at least part of the hinge region. In general, an Fc polypeptide does not contain a variable region.

A “modified Fc polypeptide” refers to an Fc polypeptide that has at least one mutation, e.g., a substitution, deletion or insertion, as compared to a wild-type immunoglobulin heavy chain Fc polypeptide sequence, but retains the overall Ig fold or structure of the native Fc polypeptide.

The term “variable region” or “variable domain” refers to a domain in an antibody heavy chain or light chain that is derived from a germline Variable (V) gene, Diversity (D) gene, or Joining (J) gene (and not derived from a Constant (Cμ and Cδ) gene segment), and that gives an antibody its specificity for binding to an antigen. Typically, an antibody variable region comprises four conserved “framework” regions interspersed with three hypervariable “complementarity determining regions.”

The terms “antigen-binding portion” and “antigen-binding fragment” are used interchangeably herein and refer to one or more fragments of an antibody that retains the ability to specifically bind to an antigen via its variable region. Examples of antigen-binding fragments include, but are not limited to, a Fab fragment (a monovalent fragment consisting of the VL, VH, CL, and CH1 domains), a F(ab′)₂ fragment (a bivalent fragment comprising two Fab fragments linked by a disulfide bridge at the hinge region), a single chain Fv (scFv), a disulfide-linked Fv (dsFv), complementarity determining regions (CDRs), a VL (light chain variable region), and a VH (heavy chain variable region).

As used herein, the terms “ETV:IDS,” “ETV:IDS protein,” or “ETV:IDS fusion protein” refer to a protein comprising a first Fc polypeptide that is linked to an IDS enzyme, IDS enzyme variant, or a catalytically active fragment thereof, and a second Fc polypeptide; wherein the first and/or second Fc polypeptide is a modified Fc that is capable of binding (e.g., specifically binding) to a TfR (i.e., the Fc comprises a TfR binding domain described herein). The ETV:IDS fusion protein may also be further processed during cell culture production, such that the C′terminal Lys residue is removed or not present in one or both of the Fc polypeptides (i.e., the Lys residue at position 447, according to the EU numbering scheme). Thus, as used herein, the terms “ETV:IDS”, “ETV:IDS protein,” or “ETV:IDS fusion protein” may refer to protein molecules having unprocessed sequences or to protein molecules comprising one or more processed sequences. A pharmaceutical composition containing ETV:IDS can include a mixture comprising processed and unprocessed protein molecules. A particular ETV:IDS embodiment, referred to as ETV:IDS 35.23.2 protein, is a dimer formed by an IDS-Fc fusion polypeptide comprising the sequence of any one of SEQ ID NOS:35, 36, and 37 and a second Fc polypeptide that specifically binds to TfR and comprises the sequence of SEQ ID NO:29 (see, CAS Registry No. 2641020-57-5). This ETV:IDS 35.23.2 fusion protein may also be further processed during cell culture production, such that the IDS-Fc fusion polypeptide comprises the sequence of any one of SEQ ID NOS: 67, 68, and 69 and/or the second Fc polypeptide that specifically binds to TfR comprises the sequence of SEQ ID NO:56. Thus, as used herein, the terms “ETV:IDS 35.23.2”, “ETV:IDS 35.23.2 protein,” or “ETV:IDS 35.23.2 fusion protein” may refer to protein molecules having unprocessed sequences (i.e., SEQ ID NOs:35, 36, 37, and 29) or to protein molecules comprising one or more processed sequences (i.e., selected from SEQ ID NOs: 67, 68, 69, and 56). A pharmaceutical composition containing ETV:IDS 35.23.2 can include a mixture comprising processed and unprocessed protein molecules.

The terms “CH3 domain” and “CH2 domain” as used herein refer to immunoglobulin constant region domain polypeptides. For purposes of this application, a CH3 domain polypeptide refers to the segment of amino acids from about position 341 to about position 447 as numbered according to EU, and a CH2 domain polypeptide refers to the segment of amino acids from about position 231 to about position 340 as numbered according to the EU numbering scheme and does not include hinge region sequences. CH2 and CH3 domain polypeptides may also be numbered by the IMGT (ImMunoGeneTics) numbering scheme in which the CH2 domain numbering is 1-110 and the CH3 domain numbering is 1-107, according to the IMGT Scientific chart numbering (IMGT website). CH2 and CH3 domains are part of the Fc region of an immunoglobulin. An Fc region refers to the segment of amino acids from about position 231 to about position 447 as numbered according to the EU numbering scheme, but as used herein, can include at least a part of a hinge region of an antibody.

The terms “wild-type,” “native,” and “naturally occurring” with respect to a CH3 or CH2 domain are used herein to refer to a domain that has a sequence that occurs in nature.

As used herein, the term “mutant” with respect to a mutant polypeptide or mutant polynucleotide is used interchangeably with “variant.” A variant with respect to a given wild-type CH3 or CH2 domain reference sequence can include naturally occurring allelic variants. A “non-naturally” occurring CH3 or CH2 domain refers to a variant or mutant domain that is not present in a cell in nature and that is produced by genetic modification, e.g., using genetic engineering technology or mutagenesis techniques, of a native CH3 domain or CH2 domain polynucleotide or polypeptide. A “variant” includes any domain comprising at least one amino acid mutation with respect to wild-type. Mutations may include substitutions, insertions, and deletions.

The term “amino acid” refers to naturally occurring and synthetic amino acids, as well as amino acid analogs and amino acid mimetics that function in a manner similar to the naturally occurring amino acids.

Naturally occurring amino acids are those encoded by the genetic code, as well as those amino acids that are later modified, e.g., hydroxyproline, γ-carboxyglutamate and O-phosphoserine. “Amino acid analogs” refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that function in a manner similar to a naturally occurring amino acid.

Naturally occurring a-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally-occurring a-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Amino acids may be referred to herein by either their commonly known three letter symbols or by the one-letter symbols recommended by the IUPAC-IUB Biochemical Nomenclature Commission.

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L and D amino acids.

The term “protein” as used herein refers to either a polypeptide or a dimer (i.e, two) or multimer (i.e., three or more) of single chain polypeptides. The single chain polypeptides of a protein may be joined by a covalent bond, e.g., a disulfide bond, or non-covalent interactions.

The term “conservative substitution,” “conservative mutation,” or “conservatively modified variant” refers to an alteration that results in the substitution of an amino acid with another amino acid that can be categorized as having a similar feature. Examples of categories of conservative amino acid groups defined in this manner can include: a “charged/polar group” including Glu (Glutamic acid or E), Asp (Aspartic acid or D), Asn (Asparagine or N), Gln (Glutamine or Q), Lys (Lysine or K), Arg (Arginine or R), and His (Histidine or H); an “aromatic group” including Phe (Phenylalanine or F), Tyr (Tyrosine or Y), Trp (Tryptophan or W), and (Histidine or H); and an “aliphatic group” including Gly (Glycine or G), Ala (Alanine or A), Val (Valine or V), Leu (Leucine or L), Ile (Isoleucine or I), Met (Methionine or M), Ser (Serine or S), Thr (Threonine or T), and Cys (Cysteine or C). Within each group, subgroups can also be identified. For example, the group of charged or polar amino acids can be sub-divided into sub-groups including: a “positively-charged sub-group” comprising Lys, Arg and His; a “negatively-charged sub-group” comprising Glu and Asp; and a “polar sub-group” comprising Asn and Gln. In another example, the aromatic or cyclic group can be sub-divided into sub-groups including: a “nitrogen ring sub-group” comprising Pro, His and Trp; and a “phenyl sub-group” comprising Phe and Tyr. In another further example, the aliphatic group can be sub-divided into sub-groups, e.g., an “aliphatic non-polar sub-group” comprising Val, Leu, Gly, and Ala; and an “aliphatic slightly-polar sub-group” comprising Met, Ser, Thr, and Cys. Examples of categories of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free —OH can be maintained; and Gln for Asn or vice versa, such that a free —NH₂ can be maintained. In some embodiments, hydrophobic amino acids are substituted for naturally occurring hydrophobic amino acid, e.g., in the active site, to preserve hydrophobicity.

The terms “identical” or percent “identity,” in the context of two or more polypeptide sequences, refer to two or more sequences or subsequences that are the same or have a specified percentage of amino acid residues, e.g., at least 60% identity, at least 65%, at least 70%, at least 75%, at least 80%, at least 85%, at least 90%, or at least 95% or greater, that are identical over a specified region when compared and aligned for maximum correspondence over a comparison window, or designated region, as measured using a sequence comparison algorithm or by manual alignment and visual inspection.

For sequence comparison of polypeptides, typically one amino acid sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 (Genentech, South San Francisco, Calif.) or Megalign (DNASTAR). The parameters employed for an alignment to achieve maximal alignment can be determined by one of skill in the art. For sequence comparison of polypeptide sequences for purposes of this application, the BLASTP algorithm standard protein BLAST for aligning two proteins sequence with the default parameters is used.

The terms “corresponding to,” “determined with reference to,” or “numbered with reference to” when used in the context of the identification of a given amino acid residue in a polypeptide sequence, refers to the position of the residue of a specified reference sequence when the given amino acid sequence is maximally aligned and compared to the reference sequence. Thus, for example, an amino acid residue in a modified Fc polypeptide “corresponds to” an amino acid in SEQ ID NO:1, when the residue aligns with the amino acid in SEQ ID NO:1 when optimally aligned to SEQ ID NO:1. The polypeptide that is aligned to the reference sequence need not be the same length as the reference sequence.

A “binding affinity” as used herein refers to the strength of the non-covalent interaction between two molecules, e.g., a single binding site on a polypeptide and a target, e.g., transferrin receptor, to which it binds. Thus, for example, the term may refer to 1:1 interactions between a polypeptide and its target, unless otherwise indicated or clear from context. Binding affinity may be quantified by measuring an equilibrium dissociation constant (K_{D}), which refers to the dissociation rate constant (k_{d}, time⁻¹) divided by the association rate constant (k_{d}, time⁻¹ M⁻¹). K_{D} can be determined by measurement of the kinetics of complex formation and dissociation, e.g., using Surface Plasmon Resonance (SPR) methods, e.g., a Biacore™ system; kinetic exclusion assays such as KinExA®; and BioLayer interferometry (e.g., using the ForteBio® Octet® platform). As used herein, “binding affinity” includes not only formal binding affinities, such as those reflecting 1:1 interactions between a polypeptide and its target, but also apparent affinities for which K_{D} values are calculated that may reflect avid binding.

As used herein, the term “specifically binds” or “selectively binds” to a target, e.g., TfR, when referring to a TfR binding domain (e.g., comprised in an engineered TfR binding polypeptide as described herein), refers to a binding reaction whereby the TfR binding domain binds to the target with greater affinity, greater avidity, and/or greater duration than it binds to a structurally different target. The term “specific binding,” “specifically binds to,” or “is specific for” a particular target (e.g., TfR), as used herein, can be exhibited, for example, by a molecule having an equilibrium dissociation constant K_{D} for the target to which it binds of, e.g., 10⁻⁶ M, 10⁻⁷ M, 10⁻⁸ M, or 10⁻⁹ M. In some embodiments, a TfR binding domain (e.g., comprised in an engineered TfR binding polypeptide) may bind exclusively to a human TfR.

As used herein, the term “transferrin receptor binding domain” or “TfR binding domain” refers to a peptide or protein, or fragment thereof, that is capable of specifically binding to a transferrin receptor protein. Such TfR binding domains may be used to facilitate delivery of a peptide or protein (e.g., a protein comprising an IDS enzyme, IDS enzyme variant, or a catalytically active fragment thereof), across the blood-brain barrier (BBB). Exemplary TfR binding domains include full-length antibodies with variable regions that specifically bind to a transferrin receptor and antibody fragments containing all or a portion of a TfR-specific antigen-binding domain, such as those described in US 2016/0369001, US 2018/0171012, and US 2019/0338043. For example, a TfR-binding antibody can comprise an antibody heavy chain sequence of SEQ ID NO:251 and an antibody light chain sequence of SEQ ID NO:196 as described in US 2018/0171012, or it can comprise the immunoglobulin portion of the entity defined by CAS Registry No. 2140211-48-7. Additional exemplary TfR binding domains can also include modified Fc domains in which a TfR-binding epitope is inserted, such as those described in WO 2019/070577.

The following Examples are intended to be non-limiting.

### Example 1: A Study to Determine the Safety, Pharmacokinetics, and Pharmacodynamics of ETV:IDS in Pediatric Subjects with Hunter Syndrome

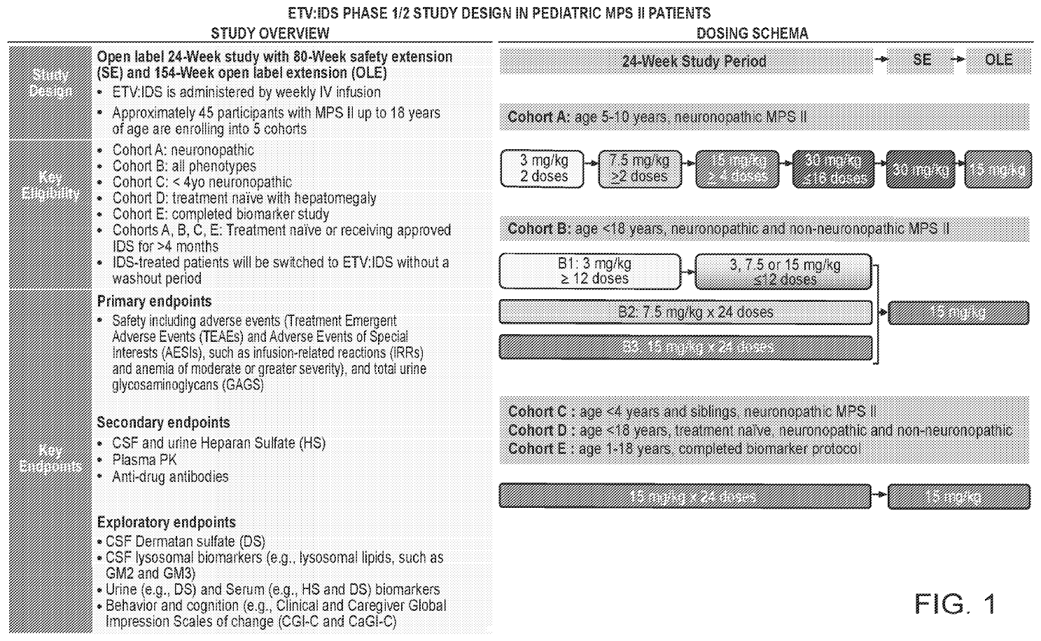

A study was performed to assess the safety, pharmacokinetics (PK), and pharmacodynamics (PD) of ETV:IDS 35.23.2 (hereinafter referred to in Example 1 as ETV:IDS), an investigational central nervous system (CNS)-penetrant enzyme replacement therapy (ERT), designed to treat both the peripheral and CNS manifestations of Hunter syndrome (MPS II) (see, FIG. 1). Patients who were on standard of care (idursulfase) enzyme replacement treatment received weekly intravenous doses of ETV:IDS on Day 1 of the study after switching from the standard of care therapy. Patients who were treatment naïve began weekly intravenous doses of ETV:IDS on Day 1 of the study.

### Results

### Safety Analysis and Analysis of Certain Biomarkers in Serum and Urine

### 1. Interim Analysis #1 for Cohorts A and B

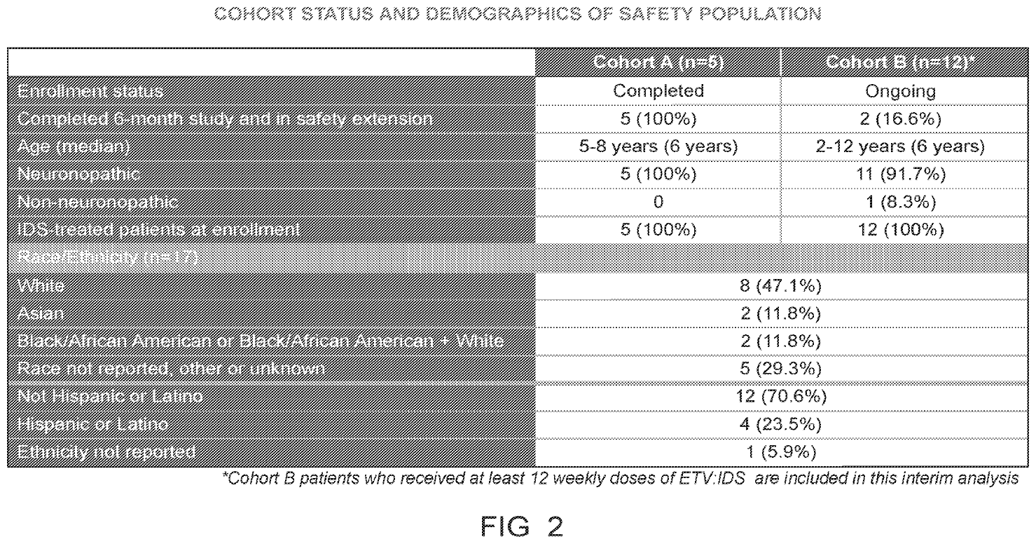

A first interim analysis of was carried out with patients in Cohort A through Week 24 of the study (i.e. after 23 weekly doses of ETV:IDS) and patients in Cohort B who received at least 12 weekly doses of ETV:IDS. Cohort status and demographics for this interim safety population are provided in FIG. 2.

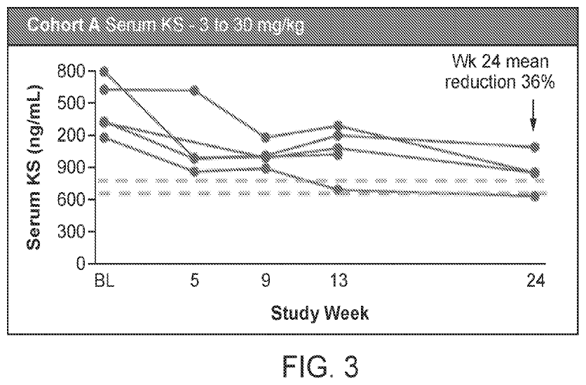

FIG. 3 provides serum keratan sulfate (KS) data for Cohort A through Week 24 of dosing with ETV:IDS. Study participants were switched from weekly idursulfase enzyme replacement therapy to weekly ETV:IDS without a washout period for idursulfase. Serum samples were obtained at baseline (e.g., prior to initiation of ETV:IDS treatment) and treatment weeks 5, 9, 13, and 24. As seen in FIG. 3, all participants show a reduction from their baseline KS values after switching from idursulfase to ETV:IDS. Treatment resulted in a mean decline of 24% in serum KS levels after four weekly doses of ETV:IDS with continued reduction observed through Week 24 (maximum mean reduction of 36% from baseline). As can be seen in FIG. 3, three out of five participants reached the normal or approached normal range of serum KS at Week 24 of treatment, and one participant achieved normalization by Week 13 of the study. This contrasts with unchanged KS levels in MIPS II patients treated with idursulfase enzyme replacement therapy or hematopoietic stem cell therapy (HSCT) (Fujitsuka et al. 2019. Mol Genet Metab Rep. 19:100455).

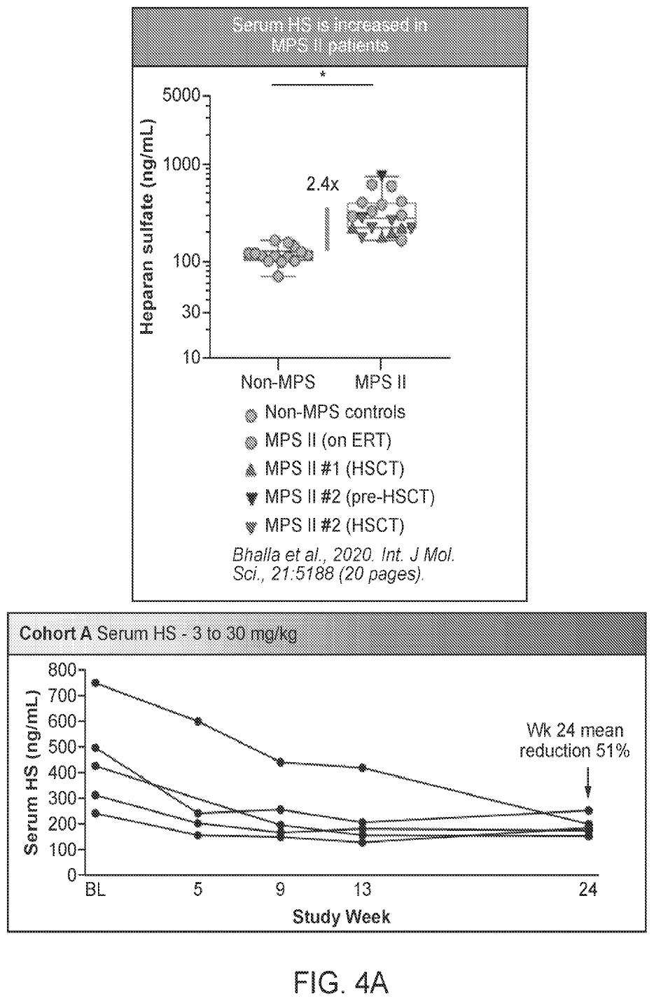

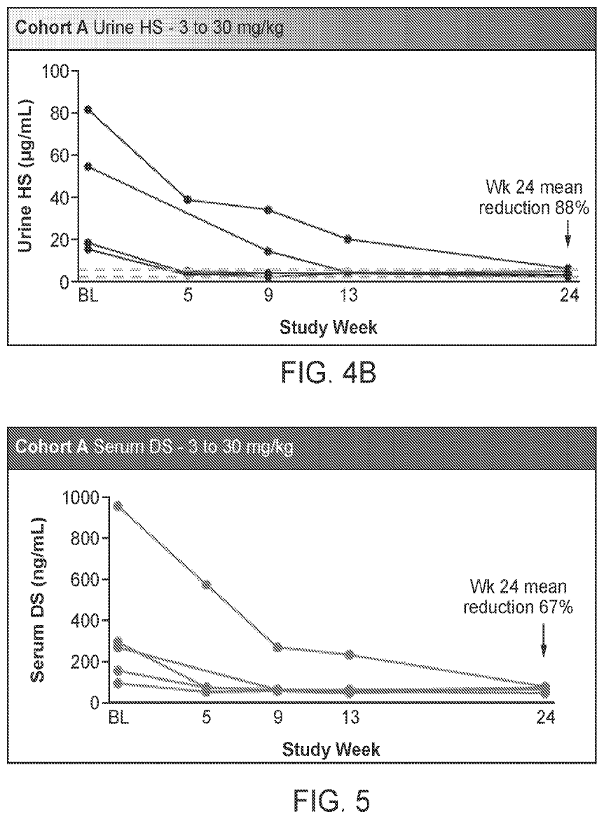

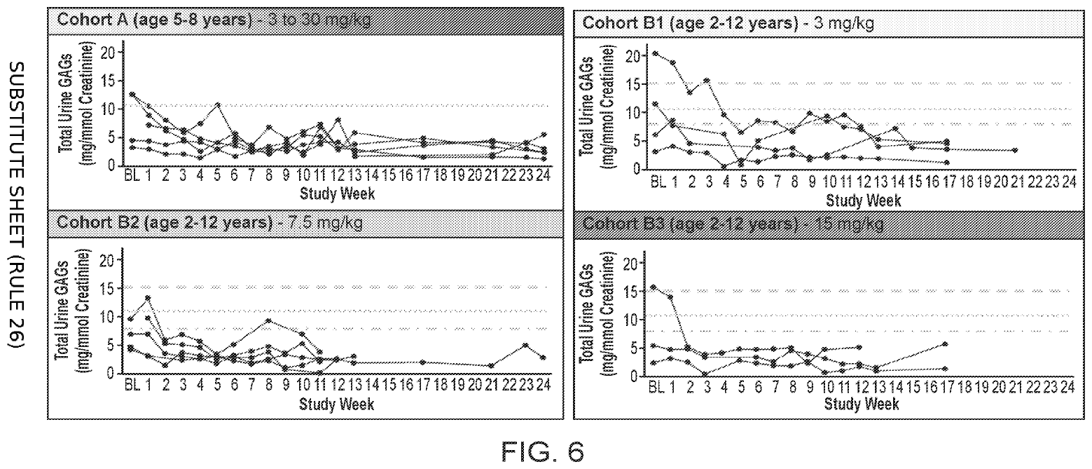

In Cohort A, after switching from idursulfase to ETV:IDS without a washout period, a decline in serum and urine heparan sulfate (HS) levels was observed (FIGS. 4A-4B). All participants in Cohort A had normal urine HS levels at Week 24, suggesting added peripheral activity. Serum dermatan sulfate (DS) levels (FIG. 5) and urine DS levels (data not shown) were also reduced following the switch. Finally, a decline in total urine glycosaminoglycans (GAGs) was observed in the majority of participants in Cohort A and participants in Cohort B who received at least 12 weekly doses of ETV:IDS (FIG. 6), suggesting added peripheral activity in all cohorts studied.

### 2. Interim Analysis #2 for Cohorts A and B

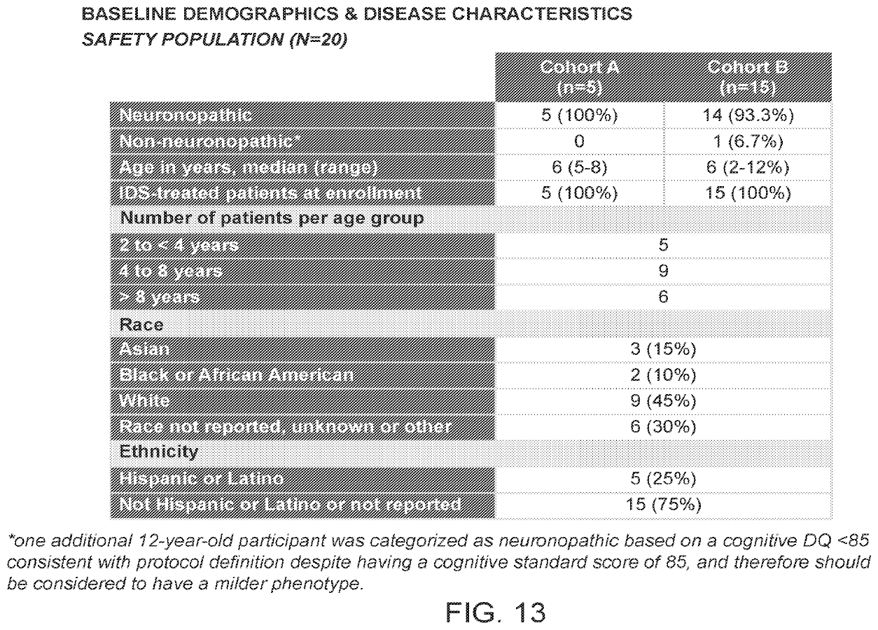

A second interim analysis was carried out with patients in Cohort A and Cohort B (i.e., Cohorts B1, B2, and B3) who had reached Week 24 of the study (i.e. after 23 weekly doses of ETV:IDS), as well as patients in Cohort B who are currently progressing towards Week 24 of the study. Cohort status and demographics for the second interim analysis are provided in FIG. 13 for the safety population.

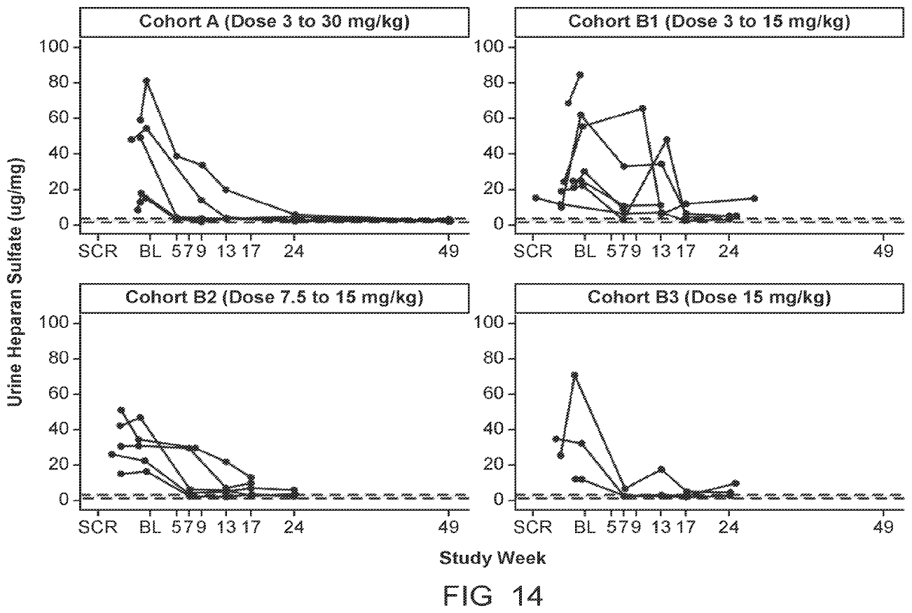

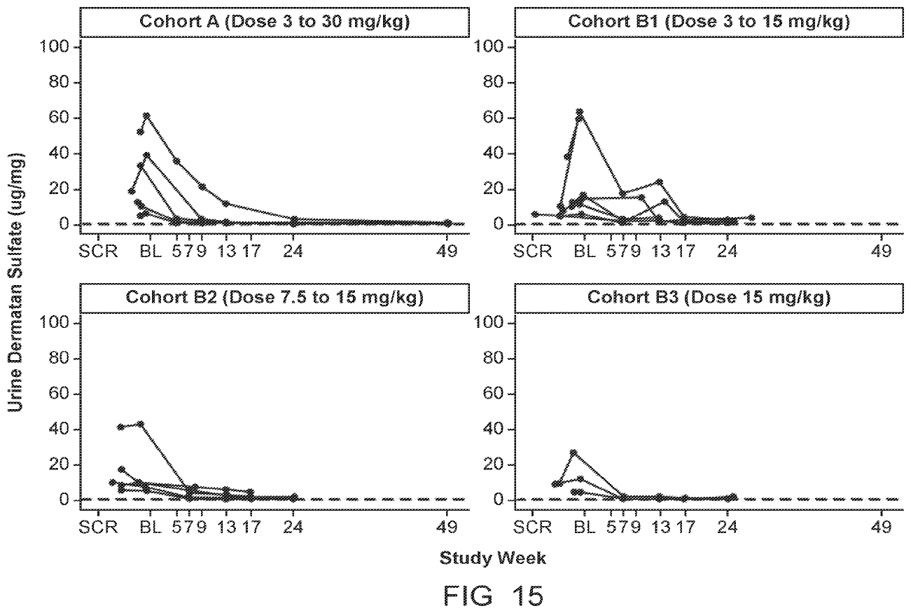

Among the patients in Cohorts A and B who were analyzed, after switching from idursulfase to ETV:IDS without a washout period, a decline in urine heparan sulfate (HS) and dermatan sulfate (DS) levels was observed (FIGS. 14, 15). At Week 24 of the study, both urine HS and DS levels exhibited a greater than 80% reduction relative to baseline levels measured prior to the treatment switch to ETV:IDS (Table 1). Several patients achieved normal or near normal levels of urine HS and DS at Week 24 (FIGS. 14, 15). Within Cohort A, both urine HS and DS levels exhibited a greater than 90% reduction relative to baseline levels measured prior to the treatment switch to ETV:IDS (Table 1). The normalization or near normalization of urine HS and DS levels observed in Cohort A at Week 24 was sustained at Week 49 (i.e. after 48 weekly doses of ETV:IDS). The observed decline in urine HS and DS levels to normal levels after the treatment switch to ETV:IDS at Week 24 among Cohorts A and B, as well as the sustained normalization of urine HS and DS levels among Cohort A at Week 49, is consistent with improved peripheral activity for ETV:IDS compared to standard of care enzyme replacement therapy.

TABLE 1
Urine HS and DS Data (Cohorts A and B)

| | Number of | Mean HS | Mean DS |
|---|---|---|---|
| | Patients | reduction | reduction |
| Week of Study | Analyzed (N) | from baseline | from baseline |
| Week 24 | 15 | 81.5% | 86.4% |
| Week 49 | 5 | 90.4% | 94.1% |

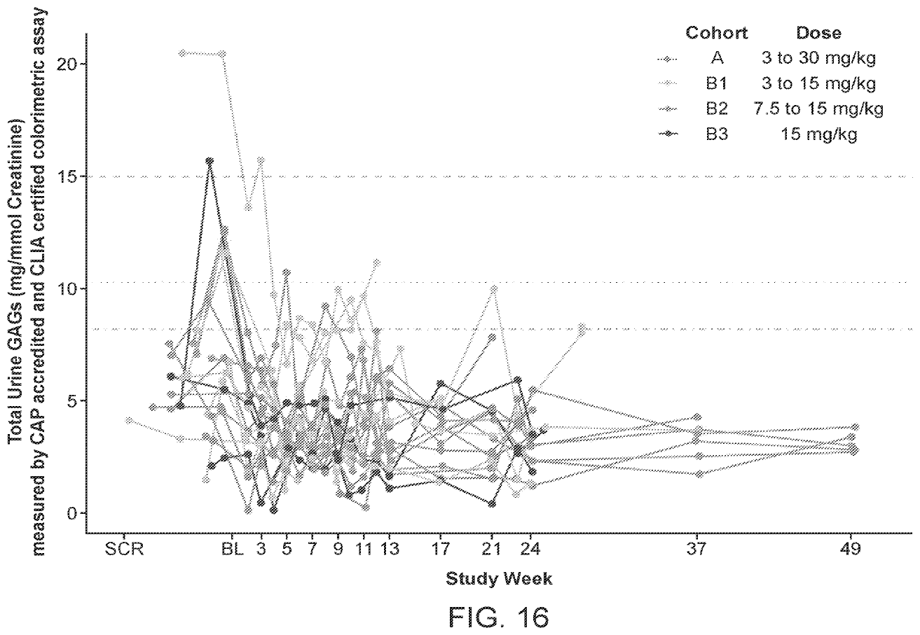

In the safety population (all patients who received any amount of ETV:IDS, n=20), total urine glycosaminoglycan (GAG) levels declined in a majority of patients after switching from idursulfase standard of care treatment to ETV:IDS treatment (FIG. 16). Upper limit of normal (ULN) of total urine GAGs/mmol creatinine is age dependent and range from 36 mg at ≤1 year to 5.5 mg at ≥14 years. Relevant ULN for each Cohort are represented by gray lines: 2-3 years ( [symbol] )<15.0 mg gags/mmol creatinine; 6-7 years ( [symbol] )<10.3 mg gags/mmol creatinine; and 10-11 years ( [symbol] )<8.2 mg gags/mmol creatinine.

### 3. Updated Analysis (Cohorts A, B, C, and D)

An updated analysis was carried out on patients from Cohorts A, B, C, and D. The updated safety analysis was based on all participants who received any amount of ETV:IDS in the study (n=27) and included patients from Cohorts A through D. The updated biomarker analysis was based on all participants who had provided CSF samples at Week 24 of the study (n=21) and included patients from Cohorts A and B (i.e., Cohorts B1, B2, and B3).

Cohort status and demographics are provided in FIG. 21 for the safety analysis population. Prior to enrollment in the study, six (6) participants (including two treatment naïve participants) had elevated total urine GAG levels, and all experienced normalization of total urine GAG levels with treatment of ETV:IDS. As observed in interim analysis #1 and #2, total urine GAG levels declined following the switch from idursulfase standard of care treatment to ETV:IDS treatment. Furthermore, following the switch, total urine GAG levels were generally within the normal range. (Upper limit of normal (ULN) of total urine GAGs/mmol creatinine is age dependent and ranges from 36 mg at ≤1 year to 5.5 mg at ≥14 years.) Following the switch from idursulfase to ETV:IDS (without a washout period), a decline in total urine GAGs in the majority of participants suggests added peripheral IDS activity in all cohorts studied.

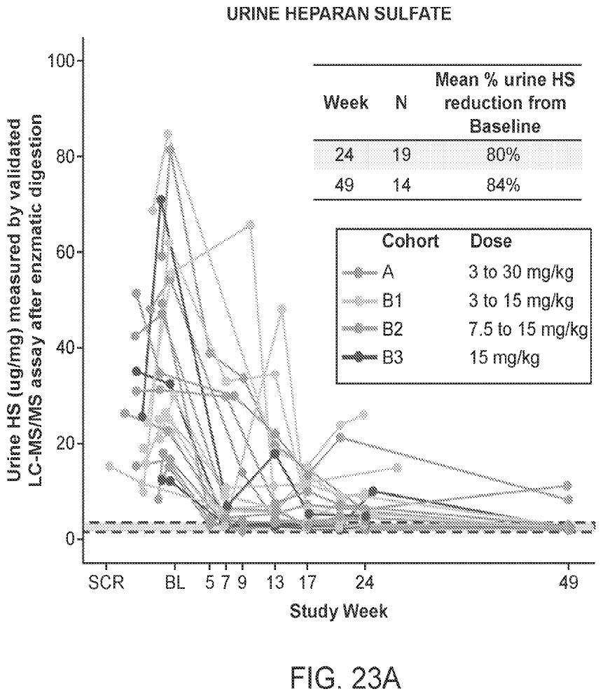

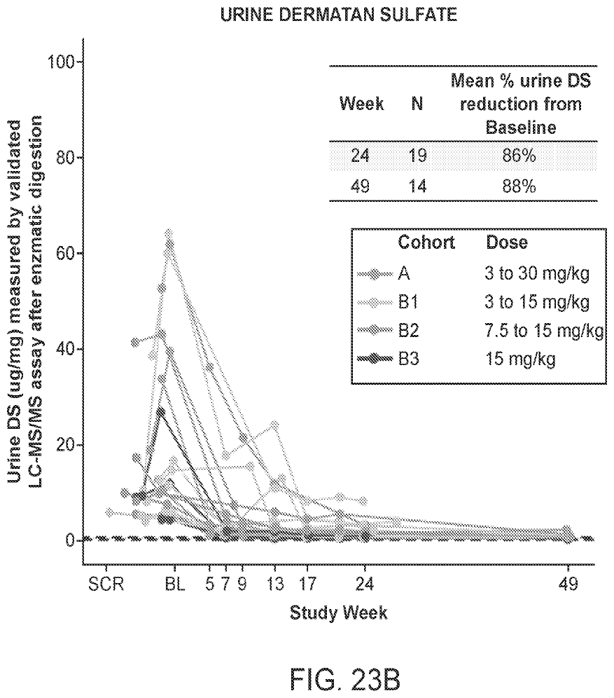

Among the patients in Cohorts A and B included in the biomarker analysis population, after switching from idursulfase to ETV:IDS without a washout period, a decline in urine heparan sulfate (HS) and dermatan sulfate (DS) levels was observed (FIGS. 23A, 23B). At Week 24 of the study, both urine HS and DS levels exhibited at least an 80% reduction relative to baseline levels measured prior to the treatment switch to ETV:IDS (Table 2). Several patients achieved normal or near normal levels of urine HS and DS at Week 24 (FIGS. 23A, 23B), which was sustained out to Week 49 of the study. The observed decline in urine HS and DS levels after the treatment switch to ETV:IDS is consistent with improved peripheral activity for ETV:IDS compared to standard of care enzyme replacement therapy.

TABLE 2
Urine HS and DS Data (Cohorts A and B)

| | Number of | Mean HS | Mean DS |
|---|---|---|---|
| | Patients | reduction | reduction |
| Week of Study | Analyzed (N) | from baseline | from baseline |
| Week 24 | 19 | 80% | 86% |
| Week 49 | 14 | 84% | 88% |

In Cohort A, serum HS levels continued to decline after one year of treatment (through Week 49 of the study), while serum DS levels were reduced by Week 24 of the study and remained stable through one year of treatment. In Cohort B, serum HS and DS levels were reduced after switching from idursulfase standard of care treatment to ETV:IDS treatment. At Week 49 of the study, overall reductions of 56% in serum HS levels and 63% in serum DS levels was observed among the patients in Cohorts A and B included in the biomarker analysis population. Table 3 provides mean percent changes in serum HS and DS levels for the various cohorts relative to baseline levels measured prior to treatment with ETV:IDS.

TABLE 3
Serum HS and DS Data (Cohorts A and B)

| | W7 | W13 | W24 | W49 |
|---|---|---|---|---|
| All | | | | |
| Dermatan | | | | |
| Sulfate (DS) | | | | |
| n | 14 | 18 | 20 | 16 |
| Mean % | −50.4 (16.5) | −57.6 (14.8) | −59.6 (18.5) | −63.1 (17.6) |
| Change (SD) | | | | |
| Heparan | | | | |
| Sulfate (HS) | | | | |
| n | 14 | 18 | 20 | 16 |
| Mean % | −39.8 (22.0) | −45.4 (12.5) | −47.3 (20.1) | −56.1 (18.7) |
| Change (SD) | | | | |
| Cohort A | | | | |
| Dermatan | | | | |
| Sulfate (DS) | | | | |
| n | 0 | 5 | 5 | 5 |
| Mean % | NA (NA) | −69.3 (13.0) | −66.8 (27.3) | −65.9 (25.7) |
| Change (SD) | | | | |
| Heparan | | | | |
| Sulfate (HS) | | | | |
| n | 0 | 5 | 5 | 5 |
| Mean % | NA (NA) | −51.0 (9.4) | −50.9 (19.3) | −67.2 (10.1) |
| Change (SD) | | | | |
| Cohort B1 | | | | |
| Dermatan | | | | |
| Sulfate (DS) | | | | |
| n | 7 | 7 | 8 | 5 |
| Mean % | −52.5 (14.6) | −55.9 (15.3) | −57.7 (17.7) | −65.0 (17.2) |
| Change (SD) | | | | |
| Heparan | | | | |
| Sulfate (HS) | | | | |
| n | 7 | 7 | 8 | 5 |
| Mean % | −49.6 (23.3) | −47.4 (14.3) | −53.1 (25.6) | −54.2 (28.4) |
| Change (SD) | | | | |
| Cohort B2 | | | | |
| Dermatan | | | | |
| Sulfate (DS) | | | | |
| n | 5 | 4 | 5 | 4 |
| Mean % | −44.7 (21.7) | −45.8 (7.8) | −57.4 (15.3) | −55.7 (9.7) |
| Change (SD) | | | | |
| Heparan | | | | |
| Sulfate (HS) | | | | |
| n | 5 | 4 | 5 | 4 |
| Mean % | −27.8 (15.8) | −35.9 (4.5) | −42.0 (8.7) | −47.3 (8.2) |
| Change (SD) | | | | |
| Cohort B3 | | | | |
| Dermatan | | | | |
| Sulfate (DS) | | | | |
| n | 2 | 2 | 2 | 2 |
| Mean % | −57.6 (9.2) | −58.0 (14.8) | −54.8 (5.9) | −66.1 (15.1) |
| Change (SD) | | | | |
| Heparan | | | | |
| Sulfate (HS) | | | | |
| n | 2 | 2 | 2 | 2 |
| Mean % | −35.5 (24.3) | −43.5 (21.4) | −28.5 (11.0) | −50.4 (19.1) |
| Change (SD) | | | | |

In conclusion, following the switch from idursulfase to ETV:IDS (without a washout period), participants in the study experienced one or more of: a reduction and/or normalization of serum keratan sulfate (KS) levels, a decline in total urine GAGs, normalization of urine heparan sulfate (HS) and dermatan sulfate (DS) levels, and reduction of serum HS and serum DS levels. The totality of data indicates added peripheral activity for ETV:IDS relative to standard of care enzyme replacement therapy in all cohorts studied.

### Global Impression of Change and Analysis of Certain Biomarkers in CSF

### Data Based on Clinician and Parent/Caregiver Global Impression of Change (CGI-C, PGI-C), Version 2

Cohort status and demographics for a safety population for interim analysis #1 of the study are provided in FIG. 2. Cohort status and demographics for the safety population for interim analysis #2 are provided in FIG. 13.

The change in the subject's condition were graded on a 7-point scale: 1=very much improved, 2=much improved, 3=minimally improved, 4=no change, 5=minimally worse, 6=much worse, and 7=very much worse.

Global Impression of Change results for Cohort A through Week 24 (Interim Analysis #1)

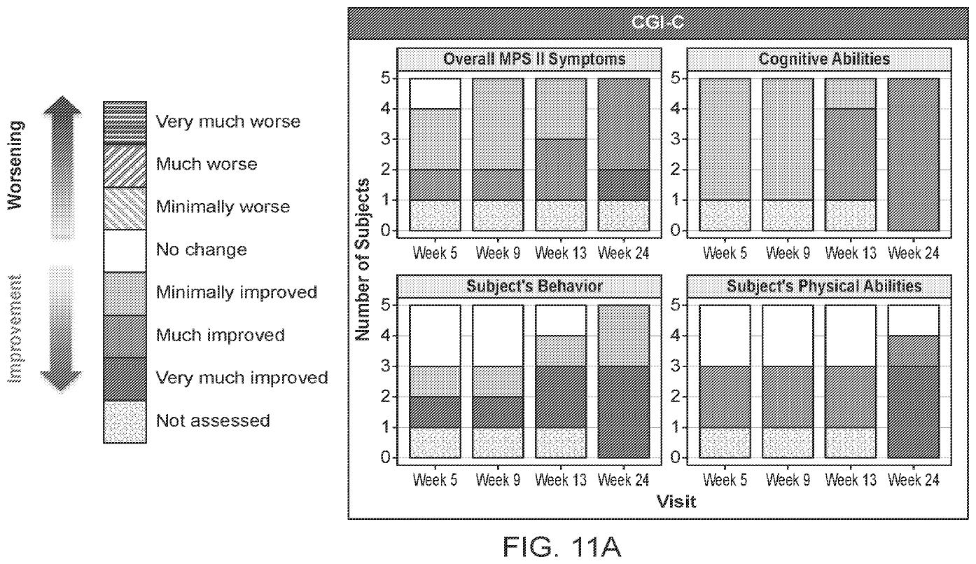

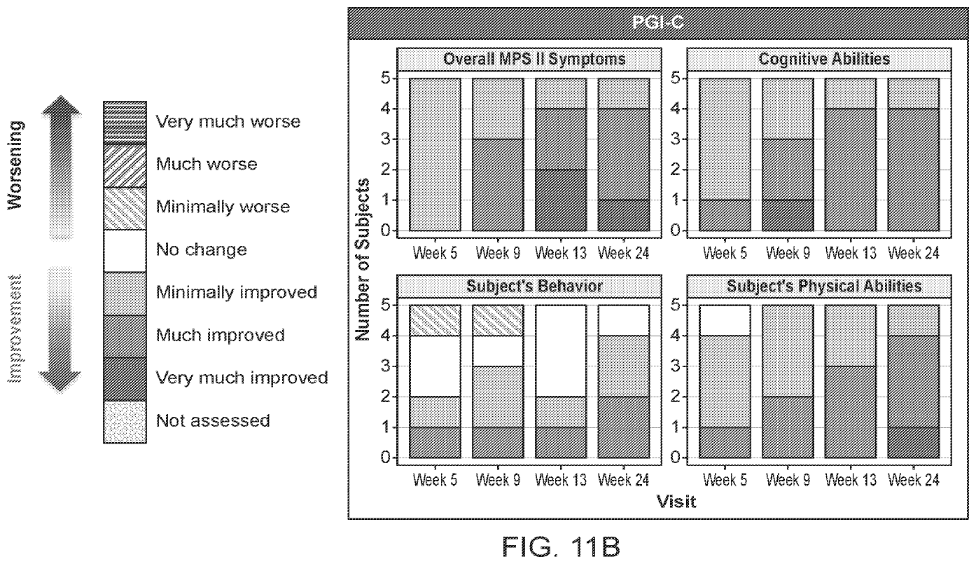

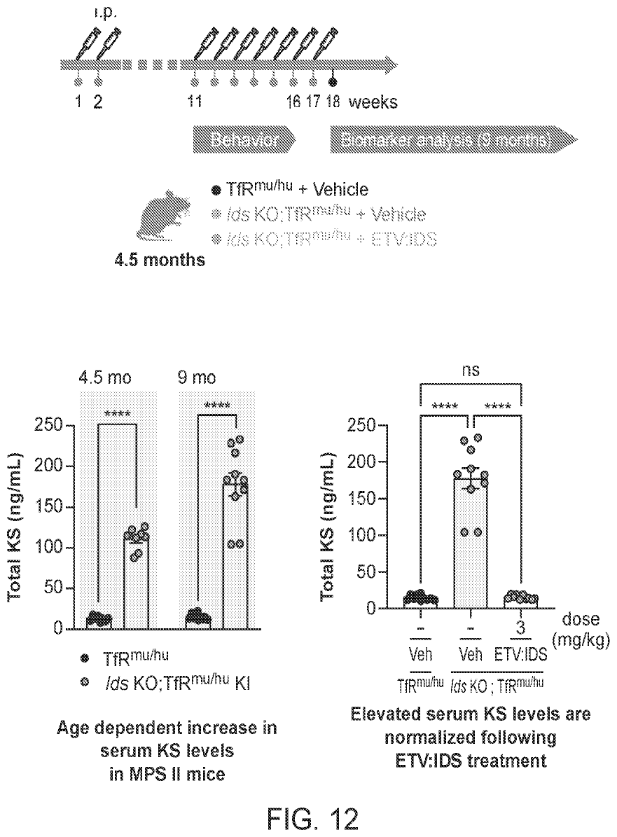

A first interim analysis of clinical outcomes was carried out on Cohort A (N=5) through Week 24 of the study (i.e., after 23 weekly doses) (FIGS. 11A, 11B). Interim assessment of clinical outcomes suggested improvement in overall MPS II symptoms, cognitive abilities, behavior, and physical abilities as assessed by an expert clinician (CGI-C) and parent/caregiver (PGI-C).

### 1. CGI-C Results for Cohort a Through Week 24

Overall MPS-II symptoms: Week 5 assessments were available for four participants in Cohort A; 1 out of 4 (25%) participants had no change, 2 out of 4 (50%) participants were minimally improved, and 1 out of 4 (25%) participants was much improved. Assessments at Week 24 were available for four Cohort A participants; 3 out of 4 (75%) participants were much improved, and 1 out of 4 (25%) participants was very much improved.

Cognitive abilities: Week 5 assessments were available for four participants in Cohort A; 4 out of 4 (100%) Cohort A participants were minimally improved. Assessments through Week 24 were available for five Cohort A participants; at Week 24, 5 out of 5 (100%) participants were much improved.

Behavior: Week 5 assessments were available for four participants in Cohort A; 2 out of 4 (50%) participants had no change, 1 out of 4 (25%) participants was minimally improved, and 1 out of 4 (25%) participants was much improved. Assessments through Week 24 were available for five Cohort A participants; at Week 24, 2 out of 5 (40%) participants were minimally improved, and 3 out of 5 (60%) participants were much improved.

Physical abilities: Week 5 assessments were available for four participants in Cohort A; 2 out of 4 (50%) participants had no change, and 2 out of 4 (50%) participants were minimally improved. Assessments at Week 24 were available for five Cohort A participants; at Week 24, 1 out of 5 (20%) participants had no change, 1 out of 5 (20%) participants was minimally improved, and 3 out of 5 (60%) participants were much improved.

### 2. PGI-C Results for Cohort a Through Week 24

Week 5 and Week 24 assessments were available for all five participants for all categories described below.

Overall MPS-II symptoms: At Week 5, all five participants (100%) were minimally improved. At Week 24, 1 out of 5 (20%) participants was minimally improved, 3 out of 5 (60%) participants were much improved, and 1 out of 5 (20%) participants was very much improved.

Cognitive abilities: At Week 5, 4 out of 5 (80%) participants were minimally improved and 1 out of 5 (20%) participants was much improved. At Week 24, 1 out of 5 (20%) participants was minimally improved, and 4 out of 5 (80%) participants were much improved.

Behavior: At Week 5, 1 out of 5 (20%) participants was minimally worse, 2 out of 5 (40%) participants had no change, 1 out of 5 (20%) participants was minimally improved, and 1 out of 5 (20%) participants was much improved. At Week 24, 1 out of 5 (20%) participants had no change, 2 out of 5 (40%) participants were minimally improved, 2 out of 5 (40%) participants were much improved.

Physical abilities: At Week 5, 1 out of 5 (20%) participants had no change, 3 out of 5 (60%) participants were minimally improved, and 1 out of 5 (20%) participants was much improved. At Week 24, 1 out of 5 (20%) participants was minimally improved, 3 out of 5 (60%) participants were much improved, and 1 out of 5 (20%) participants was very much improved.

Global Impression of Change results for Cohorts A and B through Week 24 (Interim Analysis #2)

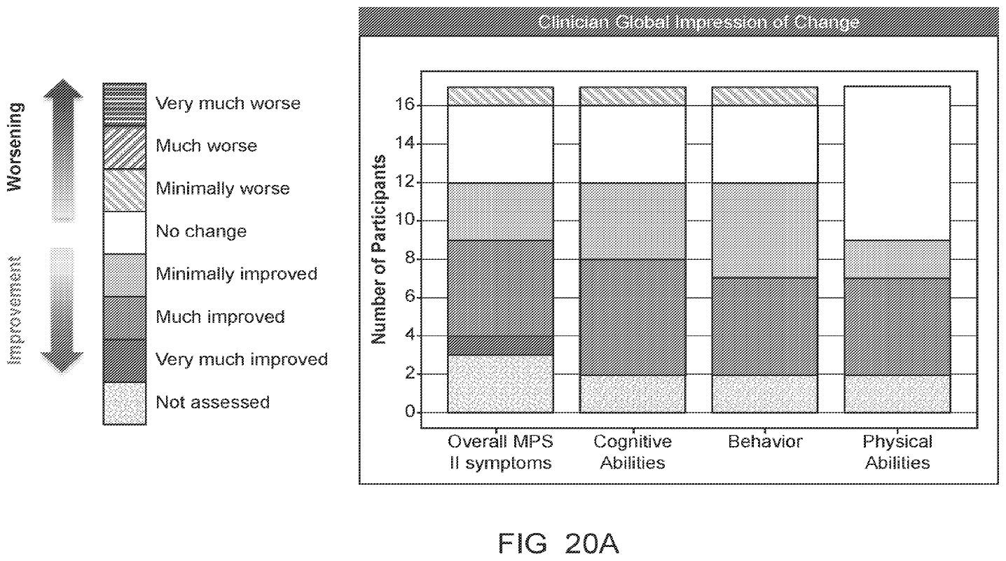

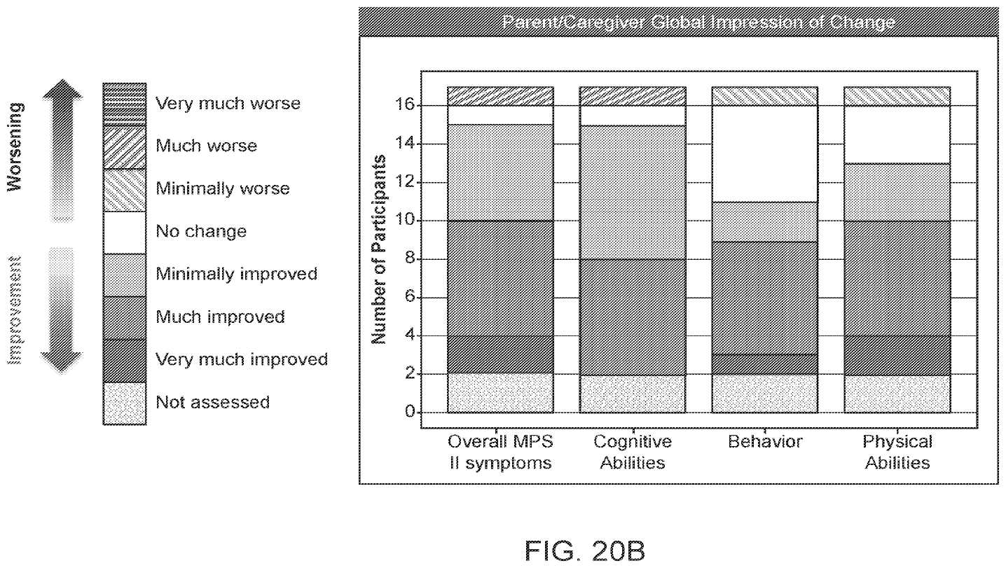

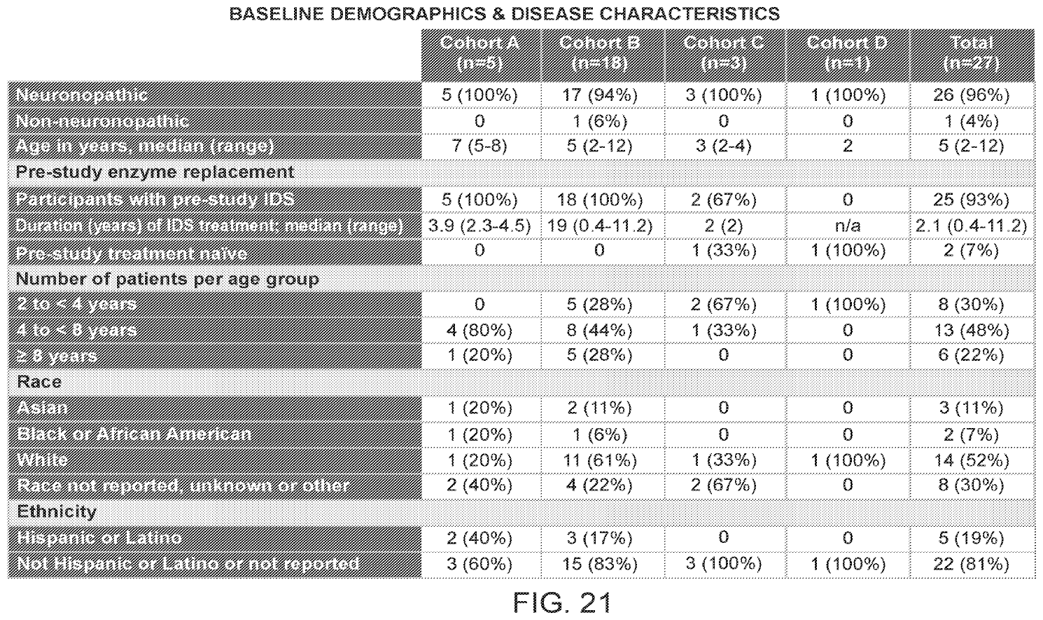

A second interim analysis of clinical outcomes was carried out on the subset of patients from Cohort A and Cohort B who completed Clinical Global Impression of Change scales at Week 24 (i.e., after 23 weekly doses of ETV:IDS; N=17) (FIGS. 20A, 20B). The results based on Clinician and Parent/Caregiver Global Impression of Change scales indicate improvement in the majority of patients in the study at Week 24. The Global Impression of Change scales are standardized assessment scales used to measure change, and the data showed clinical improvement in overall MPS II symptoms, cognitive abilities, and behavior.

### Data Based on Clinician and Caregiver Global Impression of Change (CGI-C, CaGI-C), Version 3

An updated version of the Global Impressions instrument was used to carry out an updated analysis of clinical outcomes on Cohorts A and B through Week 49. The change in the subject's condition were graded on a 5-point scale: 1=much improved, 2=a little improved, 3=no change, 4=a little worse, and 5=much worse. The subject's condition was evaluated across six domains: communication, daily living skills, overall MPS II symptoms, physical abilities, problematic behavior, and social skills.

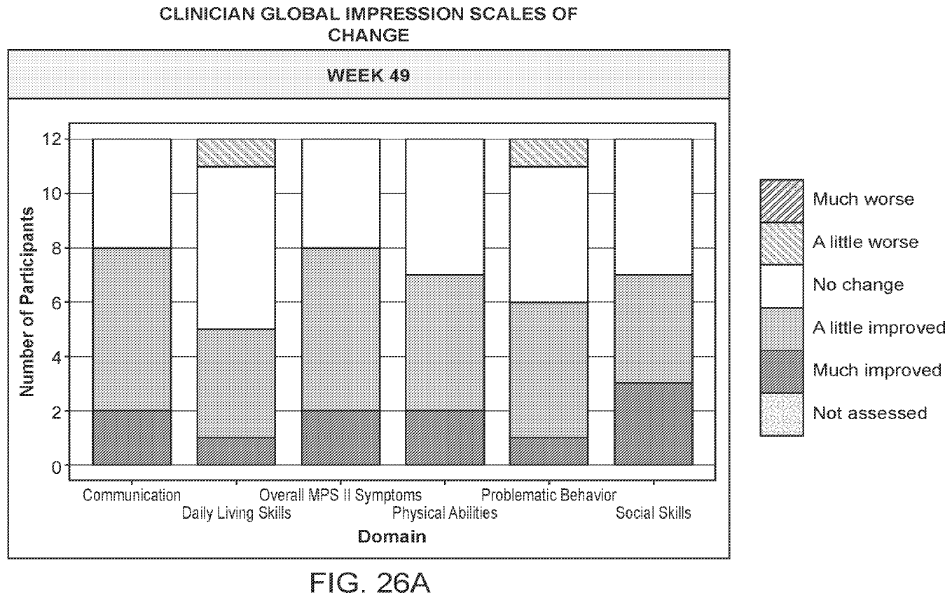

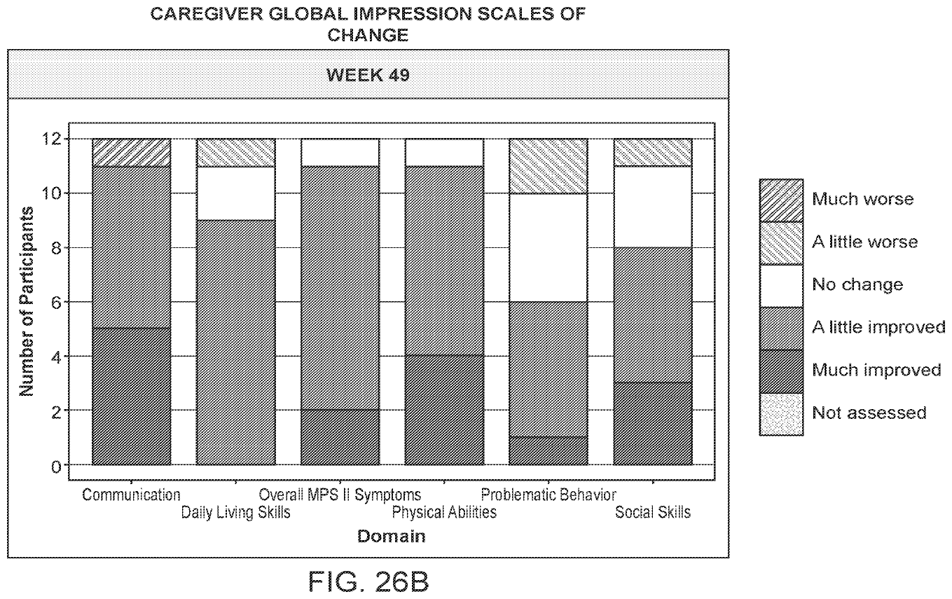

The updated analysis of clinical outcomes was carried out on the subset of participants from Cohort A and Cohort B who completed Clinical Global Impression of Change (Version 3) evaluations at Week 49 of the study (N=12) (FIGS. 26A, 26B). FIGS. 26A-26B do not include data from four participants assessed with Version 2 at Week 49; however, the data from those four participants was consistent with the data shown in the Figures. The results based on Clinician and Caregiver Global Impression of Change scales indicate that the majority of participants stabilize or improve across all domains at Week 49 of the study compared to baseline at the beginning of the study.

### CSF Biomarker Data

### 1. Interim Analysis

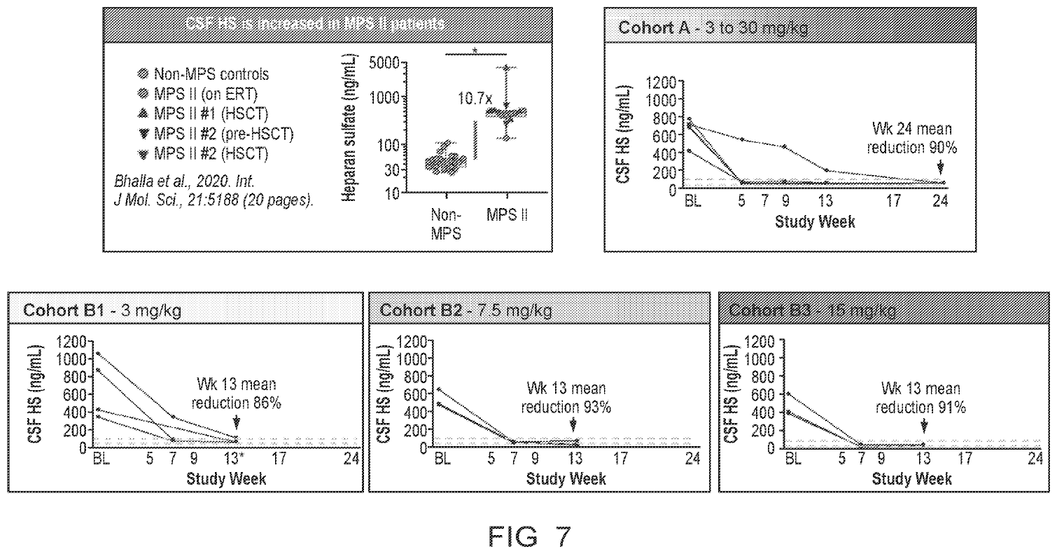

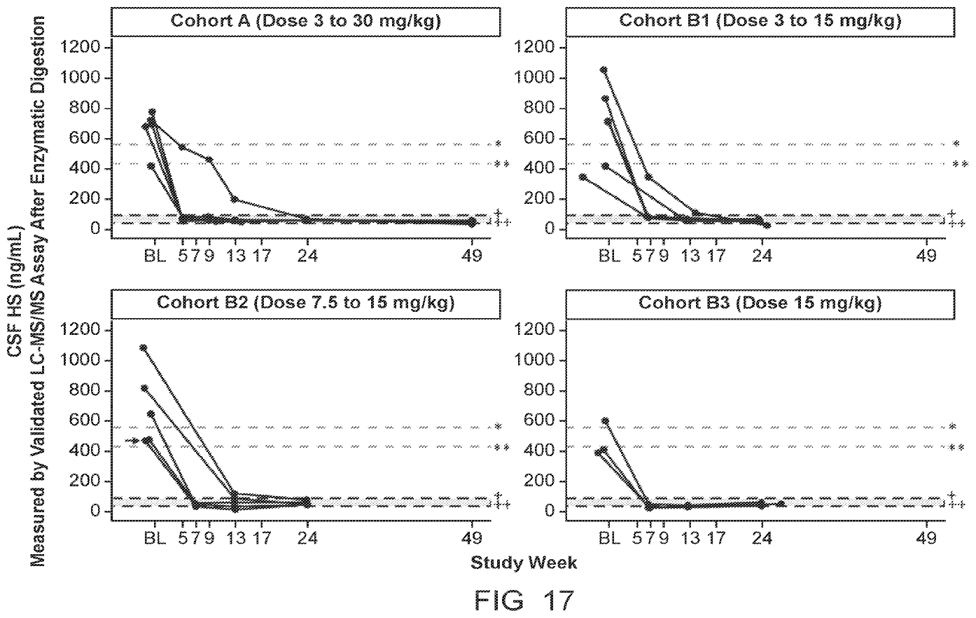

FIGS. 17-19 provide certain CSF biomarker data from interim analysis #2 based on a biomarker population (patients in Cohort A and Cohort B (i.e., Cohorts B1, B2, and B3) who had reached Week 24 of the study (i.e., after 23 weekly doses of ETV:IDS) as well as patients in Cohort B who are currently progressing towards Week 24 of the study. FIGS. 7-10 provide certain CSF biomarker data from interim analysis #1 based on a biomarker population that included patients in Cohort A through Week 24 of the study (i.e., after 23 weekly doses) and patients in Cohort B who received at least 12 weekly doses of ETV:IDS.

### A. CSF Heparan Sulfate Data

In the interim analysis #2 biomarker population, normalization of CSF heparan sulfate (HS) occurred in all patients by Week 24 of the study (i.e., after 23 weekly doses) (FIG. 17), including the non-neuronopathic MPS II patient in Cohort B2 (FIG. 17, trajectory with arrow). The normalization of CSF HS levels was sustained in the five patients in Cohort A who were analyzed at Week 49 of the study (i.e., after 48 weeks of dosing). The reduction in CSF HS levels relative to baseline levels measured prior to the treatment switch are provided in Table 4.

TABLE 4
CSF HS Data for Biomarker Population (Cohorts A and B)

| | Number of Patients | Mean HS reduction |
|---|---|---|
| Week of Study | Analyzed (N) | from baseline |
| Week 24 | 17 | 90.1% |
| Week 49* | 5 | 91.9% |

*Cohort A only

In the interim analysis #1 biomarker population, CSF HS levels normalized in all participants included in the analysis (n=15) after switching from idursulfase enzyme replacement therapy to ETV:IDS, with rapid response observed in 12 patients by Week 7 (FIG. 7). The subjects in Cohort A achieved a mean reduction in CSF HS levels relative to baseline of 90% by Week 24 of the study. The subjects in Cohorts B1, B2, and B3 achieved a mean reduction in CSF HS levels relative to baseline of 86%, 93%, and 91%, respectively, by Week 13.

### B. CSF Dermatan Sulfate Data

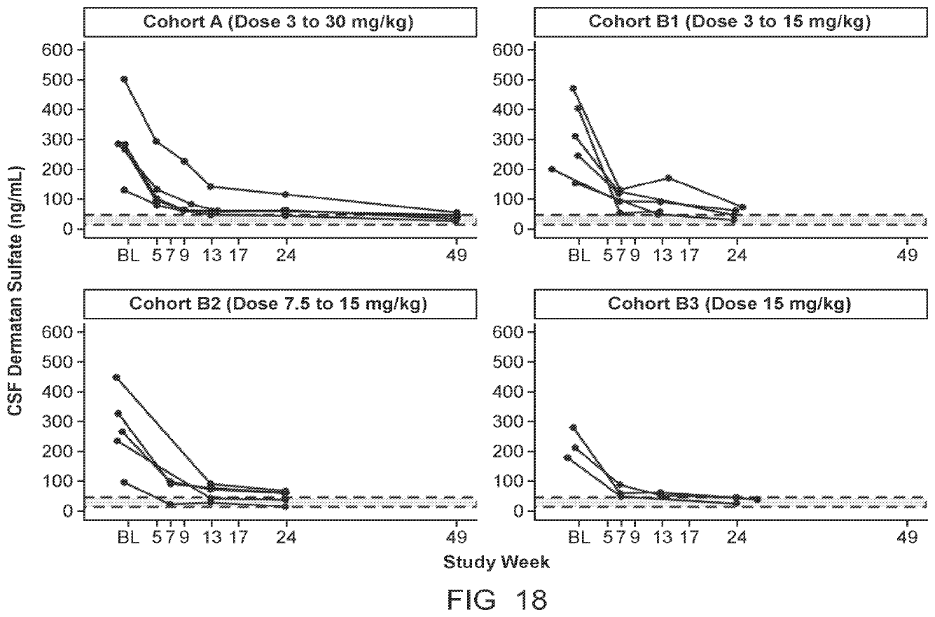

In the interim analysis #2 biomarker population, normalization or near normalization of CSF dermatan sulfate (DS) occurred in patients by Week 24 of the study (i.e., after 23 weekly doses) (FIG. 18). In those patients with normalized CSF DS levels by Week 24, sustained normalization of CSF DS levels was observed out to Week 49 of the study (i.e., after 48 weeks of dosing). The reduction in CSF DS levels relative to baseline levels measured prior to the treatment switch are provided in Table 5.

TABLE 5
CSF DS Data for Biomarker Population (Cohorts A and B)

| | Number of Patients | Mean DS reduction |
|---|---|---|
| Week of Study | Analyzed (N) | from baseline |
| Week 24 | 17 | 79.8% |
| Week 49* | 5 | 84.9% |

*Cohort A only

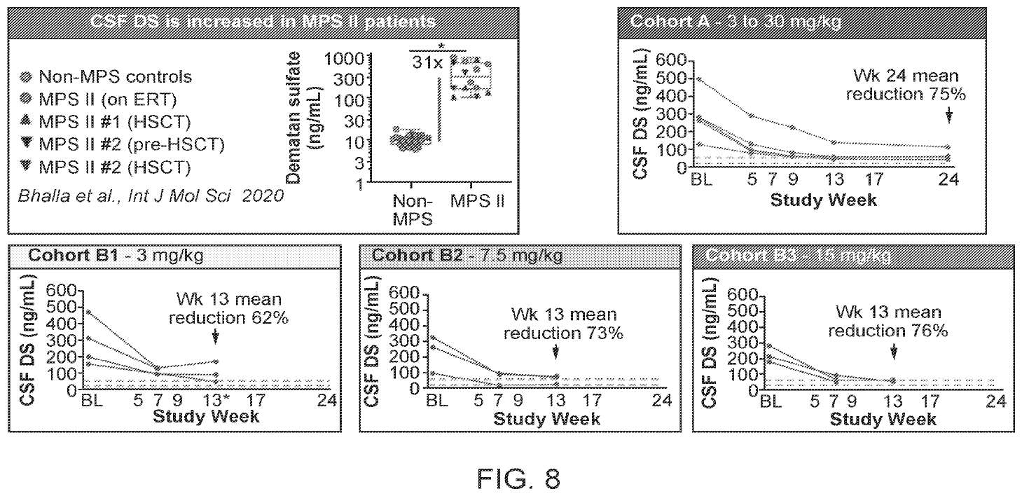

In the interim analysis #1 biomarker population, CSF DS levels normalized in most participants included in the analysis (n=15) after switching from idursulfase to ETV:IDS, with rapid response observed in 12 patients by Week 7 (FIG. 8). The subjects in Cohort A achieved a mean reduction in CSF DS levels relative to baseline of 75% by Week 24 of the study. The subjects in Cohorts B1, B2, and B3 achieved a mean reduction in CSF DS levels relative to baseline of 62%, 73%, and 76%, respectively, by Week 13.

### C. CSF Ganglioside and Exploratory Lysosomal Biomarker Data

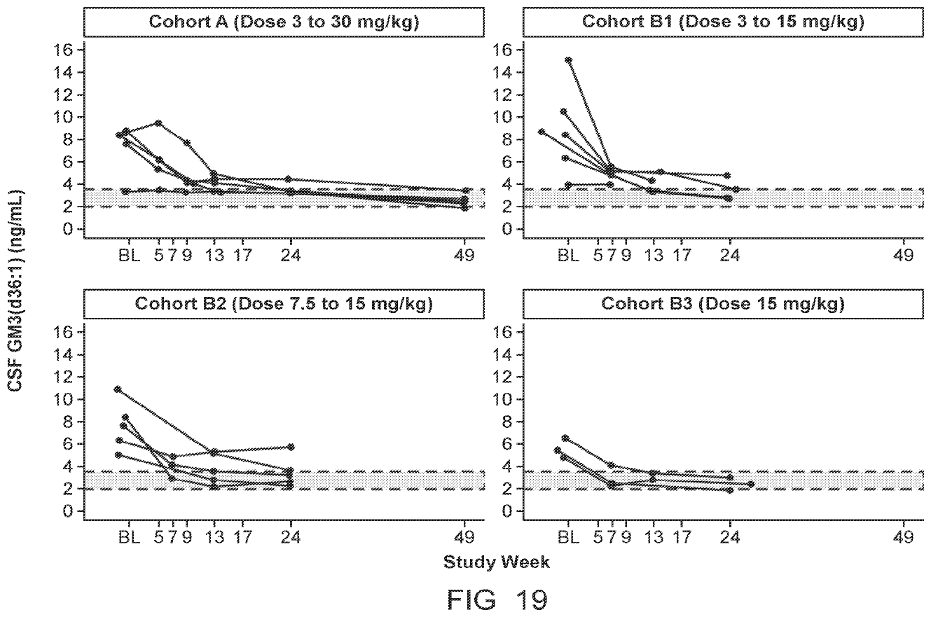

In the interim analysis #2 biomarker population, normalization CSF ganglioside levels (represented by species GM3) occurred in most patients by Week 24 of the study (i.e., after 23 weekly doses). Within the biomarker population, normalization of CSF GM3 levels was observed in all patients in Cohort A at Week 49 of the study (i.e., after 48 weeks of dosing). See FIG. 19. The reduction in CSF GM3 levels relative to baseline levels measured prior to the treatment switch are provided in Table 6. The normalization of CSF GM3 levels in most patients by Week 24 and in all patients of Cohort A at Week 49 suggests improved lysosomal function.

TABLE 6
CSF GM3 Data for Biomarker Population (Cohorts A and B)

| | Number of Patients | Mean DS reduction |
|---|---|---|
| Week of Study | Analyzed (N) | from baseline |
| Week 24 | 17 | 52.5% |
| Week 49* | 5 | 61.3% |

*Cohort A only

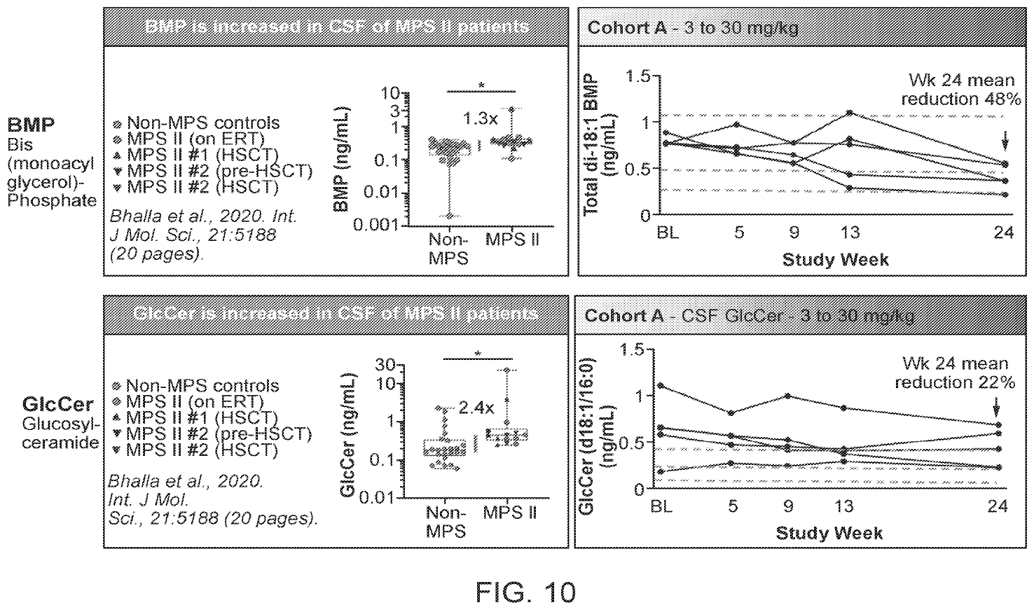

In addition, in the same biomarker population, available preliminary exploratory data on other biomarkers of lysosomal function showed reductions in CSF levels of glucosylceramide (GlcCer) across Cohorts A and B at Week 24 (Table 7) and reductions in CSF levels of bis(monoacyl glycerol)-phosphate (BMP), GM2 and glucosylsphingosine in Cohort A at Week 24 (Table 7, FIG. 10). The reduction in CSF levels of exploratory lysosomal lipid biomarkers with longer duration of ETV:IDS treatment (e.g., at least 23 weekly doses) supports improved lysosomal function in the treated patients.

TABLE 7
CSF Exploratory Lysosomal Biomarker Data

| | Mean reduction | Mean fold change compared | |
|---|---|---|---|
| | from baseline at | to healthy controls | |
| Pathway Analyte* | Week 24 | Pre-dose | Week 24 |
| GlcCer (d18:1/16:0) | 25.6% | — | — |
| BMP (total di-18:1) | 47.7% | — | — |
| GM2 (d36:1) | 47.1% | 2.6-fold | 1.2-fold |
| GM2 (d38:1) | 46.8% | 1.5-fold | 0.7-fold |
| Glucosylsphingosine | 59.6% | — | — |

*N = 17 for GlcCer (Cohorts A and B), N = 5 for all other analytes (Cohort A)

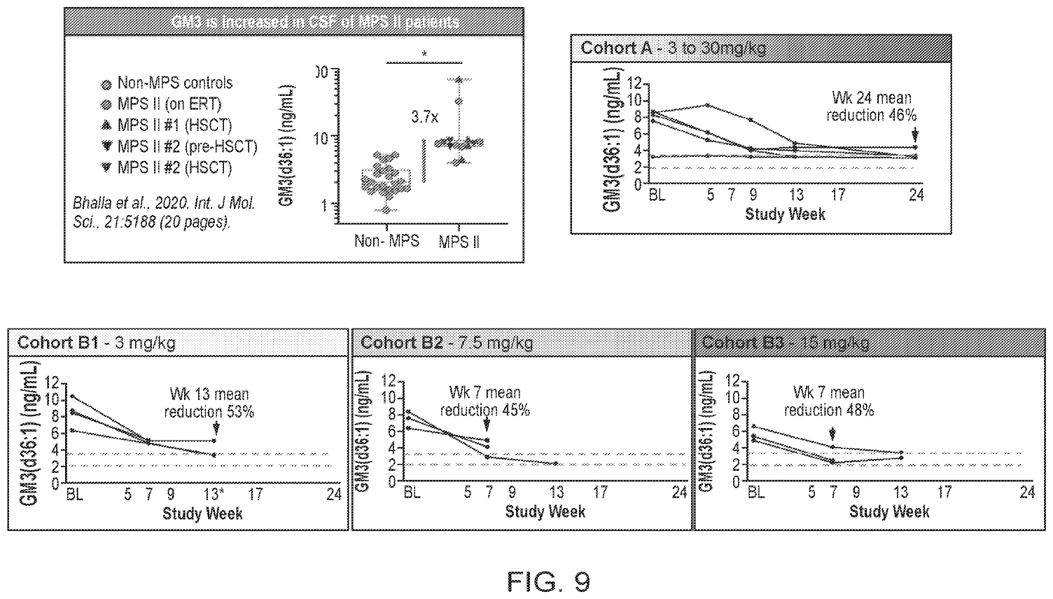

In the interim analysis #1 biomarker population, the reduction of CSF gangliosides (represented by species GM3) in dosed subjects is consistent with improved lysosomal function (FIG. 9). In cohort A, 4 of 5 participants achieved normal GM3 levels at Week 24 (i.e., after 23 weekly doses of ETV:IDS). In cohort B, 6 of 10 patients achieved normal GM3 levels to date including at lower dose regimen. The subjects in Cohort A achieved a mean reduction in CSF GM3 levels relative to baseline of 46% by Week 24 of the study. The subjects in Cohorts B1 achieved a mean reduction in CSF GM3 levels relative to baseline of 53% by Week 13, and the subjects in Cohorts B2 and B3 achieved a mean reduction in CSF GM3 levels relative to baseline of 45% and 48%, respectively, by Week 7. The reduction of CSF bis(monoacylglycerol)phosphate (BMP) and potential decline of glucosylceramide (GlcCer) at Week 24 in Cohort A is also consistent with improved lysosomal function (FIG. 10).

### 2. Updated Analysis

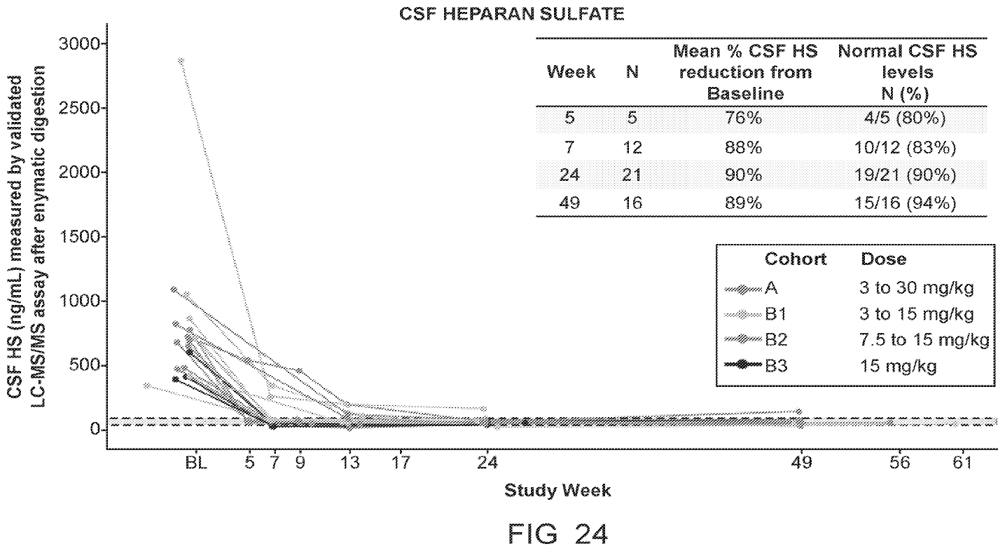

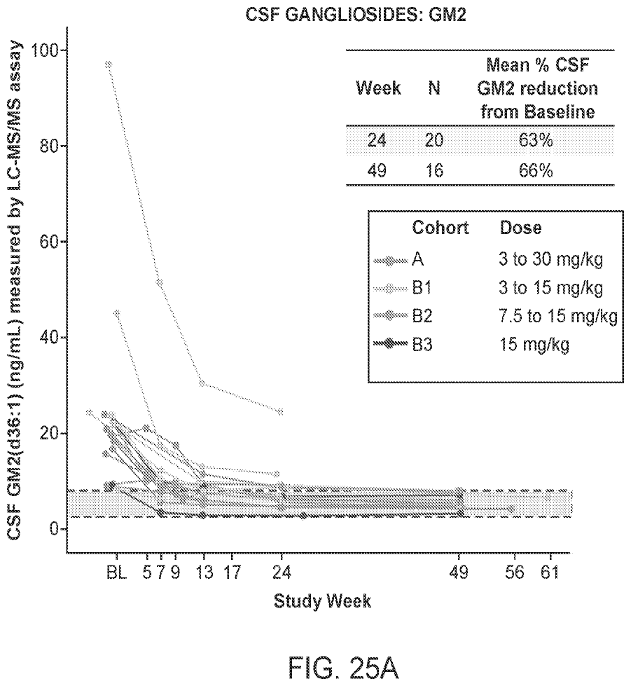

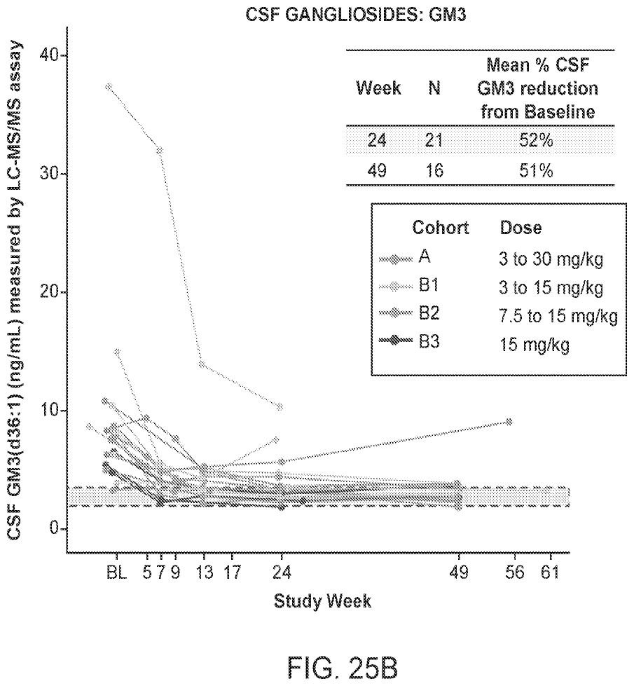

FIGS. 24 and 25 provide certain CSF biomarker data for all participants who had provided CSF samples at Week 24 of the study (N=21) and included patients from Cohorts A and B (i.e., Cohorts B1, B2, and B3).

### A. CSF Heparan Sulfate and Dermatan Sulfate Data

In the biomarker population, rapid and sustained normalization of CSF heparan sulfate (HS) levels was observed in most participants, including participants with high levels of preexisting anti-drug antibodies (ADAs) or very high baseline CSF HS levels (FIG. 24). Normalization of CSF heparan sulfate (HS) levels occurred in 19/21 participants by Week 24 of the study and in 15/16 participants by Week 49 of the study. The normalization of CSF HS levels is considered rapid because most participants achieved normal levels of CSF HS after 4 or 6 weekly doses of ETV:IDS (Table 8). Among the participants with elevated CSF HS at week 5 or 7, the participants either had very high pre-existing ADA against IDS (titer >1:10⁶) or very high CSF HS at baseline. However, even among those participants, CSF HS levels were dramatically reduced at week 5 or 7 after initiation of treatment with ETV:IDS. In addition, normalization of CSF HS levels was also achieved at the lower doses (e.g., 3 mg/kg, 7.5 mg/kg) in some participants. Finally, within the same biomarker population, a mean decline of greater than 80% from baseline was observed in CSF dermatan sulfate (DS) levels at Week 24 and sustained at Week 49 of the study (data not shown).

The reduction in CSF HS levels relative to baseline levels measured prior to the treatment switch are provided in Table 8.

TABLE 8
CSF HS Data for Biomarker Population (Cohorts A and B)

| | Number | Mean % CSF HS | Normal CSF |
|---|---|---|---|
| | of Patients | reduction from | HS levels |
| Week of Study | Analyzed (N) | baseline | N (%) |
| Week 5 | 5 | 76% | 4/5 (80%) |
| Week 7 | 12 | 88% | 10/12 (83%) |
| Week 24 | 21 | 90% | 19/21 (90%) |
| Week 49 | 16 | 89% | 15/16 (94%) |

### B. CSF Ganglioside and Exploratory Lysosomal Biomarker Data

In the biomarker population, normalization CSF ganglioside levels (represented by species GM2 and GM3) occurred in most participants by Week 24 or Week 49 of the study (FIGS. 25A, 25B). The reduction in CSF GM2 and GM3 levels relative to baseline levels measured prior to the treatment switch are provided in Table 9. The normalization of CSF GM2 and GM3 levels in most participants by Week 24 or Week 49 is consistent with improved lysosomal function. Preliminary normal ranges of GM2 and GM3 levels (10th and 90th percentile) were determined using 30 healthy adult CSF samples (age range 18-81 years, median 52 years). Normal range for CSF GM2(d36:1) (ng/mL): (2.72-8.2). Normal range for CSF GM3(d36:1) (ng/mL): (1.99-3.55).

TABLE 9
CSF GM2 and GM3 Data for Biomarker Population (Cohorts A and B)

| | Number of Patients | Mean % reduction |
|---|---|---|
| Ganglioside | Analyzed (N) | from baseline |
| GM2 | | |
| Week 24 | 20 | 63% |
| Week 49 | 16 | 66% |
| GM3 | | |
| Week 24 | 21 | 52% |
| Week 49 | 16 | 51% |

In conclusion, weekly intravenous infusions of ETV:IDS were generally well tolerated at doses of 3 mg/kg to 30 mg/kg up to 85 weeks of dosing. Both clinician and caregiver Global Impression of Change scales (CGI-C and CaGI-C) suggest that the majority of participants stabilize or improve across all category domains at Week 49 of the study compared to baseline. Rapid normalization of CSF HS levels was observed in most patients analyzed in the biomarker population, including the non-neuronopathic MPS II patient. Furthermore, sustained normalization of CSF HS levels was observed at Week 49 of the study for the biomarker population. Rapid decline of CSF DS levels with normalization or near normalization at Week 24 of the study was also sustained at Week 49. Normalization of CSF GM2 and GM3 levels in most participants and reduction in Cohort A Week 24 exploratory CSF lipid data is consistent with ETV:IDS effects on improved lysosomal function.

### Resolution of Infusion-Related Reactions in MPS II Patients Receiving ETV:IDS

Updated cohort status and demographics are provided in FIG. 21. To date, all enrolled participants are continuing in the study, and there have been no discontinuations.

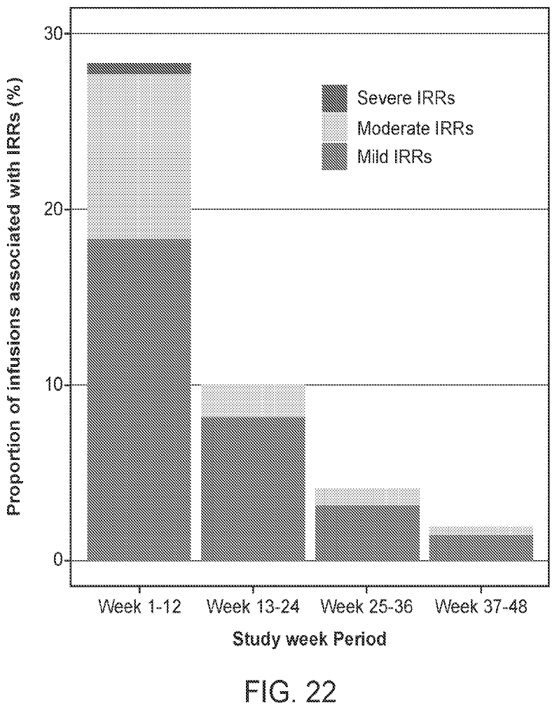

Cohort status and demographics are provided in FIG. 21 for the safety analysis population. To date, all participants reported treatment-emergent adverse events (TEAEs), with infusion related reactions (IRRs) reported as the most common TEAE, consistent with enzyme replacement therapies. However, TEAEs did not lead to any participant treatment withdrawal or study discontinuation. Among adverse events of special interest (AESIs), thirteen (13) participants experienced moderate IRRs, and one (1) participant experienced severe IRR. Among the participants with IRRs, 19/21 (90%) of participants received management consistent with standard of care for ERT. No intervention was necessary in 2/21 (10%) participants with IRR. Interestingly, the majority of IRRs occurred during the first 12 weeks of the participants' study period, and the frequency and severity of IRRs declined over time (FIG. 22).

In addition, two participants who had mild anemia at baseline experienced moderate anemia during the study. One participant's anemia resolved, and the other participant's anemia improved with continued dosing.

An earlier interim analysis was carried out on a safety population with demographic information provided in FIG. 2. All treatment-emergent adverse events (TEAEs) observed in the earlier interim analysis safety population were mild or moderate, with the exception of two (2) severe TEAEs (both infusion-related reactions) in one patient. The most common TEAE was infusion-related reactions (IRRs), which occurred in 71% of participants (12 of 17). The majority of participants with IRRs had mild (n=5) or moderate (n=6) IRRs; one participant had severe IRRs at Weeks 3 and 4 (detailed below). Of the 12 participants with IRRs, eight (8) required the following interventions to prevent subsequent IRRs: All eight (8) received standard pre-infusion medications (which included acetaminophen and diphenhydramine), and two (2) also had dose and infusion rate reductions. Most IRRs occurred between Weeks 3 and 6. Of the three patients who experienced IRRs and advanced to the safety extension (SE), most pre-infusion medications had been discontinued.

In the earlier interim safety analysis, there was one (1) serious adverse event (SAE) based on hospitalization for observation of mild IRR at Week 4 for one participant; the IRR was resolved in less than 24 hours with administration of acetaminophen and diphenhydramine. In the one participant with severe IRRs at Weeks 3 and 4, these events were classified as SAEs based on the severe IRR events meeting the Sampson criteria of anaphylaxis. The participant's IRR events were managed with pre-infusion medications and dose and infusion rate reductions. The subject has remained in the study, and subsequently has tolerated weekly doses including dose increases.

In the earlier interim safety analysis, there were no notable abnormalities or trends in safety laboratory evaluations except for anemia. Four participants (two each in Cohort A and Cohort B3) had TEAEs of anemia, all considered not related to study drug. The TEAEs of anemia were graded mild in three (3) and moderate in one (1) participant. Anemia was improved or resolved despite continued dosing at 15 mg/kg or 30 mg/kg.

All subjects who were on a standard-of-care (SOC) treatment (e.g., Elaprase or idursulfase) prior to initiation of treatment with ETV:IDS were switched to ETV:IDS treatment without a washout period upon enrollment in the study. Anti-drug antibody titer information available for Cohort A indicated that four of the five subjects had anti-drug antibodies (ADAs) against IDS at baseline, with titers ranging from 189 to greater than 11 million. Preliminary information for Cohorts B1 and B2 indicate that for participants who tested positive for ADAs at baseline, titers ranged from 63 to greater than 1 million.

In conclusion, weekly intravenous infusions of ETV:IDS were generally well tolerated at doses of 3 mg/kg to 30 mg/kg. All TEAEs reported were mild or moderate TEAEs, except for one participant who experienced two severe SAEs related to IRRs and one participant who experienced one mild SAE related to IRRs with overnight hospital observation. The most frequently observed TEAEs were IRRs. All patients are continuing in the study, and IRRs are being managed as described herein.

### Methods

### ETV:IDS

ETV:IDS 35.23.2 was produced and formulated using methods similar to those described in WO 2020/206320, which is incorporated in its entirety for all purposes.

### Study Design

The study includes Cohorts A-E: subjects with neuronopathic MPS II aged 5 to 10 years (Cohort A); subjects with MPS II, either neuronopathic or non-neuronopathic, younger than 18 years of age (Cohort B); subjects with neuronopathic MPS II who are younger than 4 years of age (Cohort C); treatment naïve subjects with MPS II, either neuronopathic or non-neuronopathic, younger than 18 years of age (Cohort D); and subjects that have completed the biomarker protocol that are aged 1 to 18 years (Cohort E) (see, FIG. 1). The study included an ascending-dose stage in Cohorts A and B to assess the safety, tolerability, PK, and PD of ETV:IDS over approximately 24 weeks. In particular, ETV:IDS is intravenously administered to the subjects weekly using the dose escalation. The duration of ETV:IDS infusion is approximately 3 hours. For Cohort A, the dose escalation of ETV:IDS was: 3 mg/kg (“Dose A,” 2 weekly infusions), 7.5 mg/kg (“Dose B,” at least 2 weekly infusions); 15 mg/kg (“Dose C,” at least 4 weekly infusions), and 30 mg/kg (“Dose D,” weekly until completion). Cohort B includes three sub-cohorts with starting doses of 3 mg/kg (B1), 7.5 mg/kg (B2), and 15 mg/kg (B3). After 24 weeks of dosing, all subjects in Cohort B receive weekly doses of 15 mg/kg thereafter (during the safety extension period). The dose for use in Cohorts C, D, and E is 15 mg/kg during the 24-week study period and in the safety extension period. For all Cohorts, the planned dose for the open label extension is 15 mg/kg.

As noted above, the study week approximates the number of doses received by the subject, wherein at week “N” of the study a subject has typically received “N−1” weekly doses; however, due to personal circumstances (e.g., illness unrelated to the treatment) a given subject may have missed one or more weekly doses during the specified period.

### Inclusion Criteria

Study participants must have a confirmed diagnosis of MPS II. Subjects enrolled in Cohort A are aged 5-10 years with neuronopathic MPS II. Subjects enrolled in Cohort B are younger than 18 years of age with non-neuronopathic MPS II, neuronopathic MPS II, or unknown phenotype. Subjects enrolling in Cohort C are younger than 4 years of age with neuronopathic MPS II and can include younger siblings of subjects in Cohort B. Subjects enrolling in Cohort D are younger than 18 years of age with non-neuronopathic or neuronopathic MPS II and are treatment naïve. Subjects enrolling in Cohort E are from 1 to 18 years of age. For subjects receiving intravenous iduronate 2-sulfastase (IDS) ERT, they are required to have tolerated a minimum of 4 months of therapy during the period immediately prior to screening.

### Exclusion Criteria

Criteria for exclusion from the study include the following conditions or events: 1) unstable or poorly controlled medical condition(s) or significant medical or psychological comorbidity or comorbidities that may interfere with safe participation in the study or interpretation of study assessments; 2) use of any CNS-targeted MPS II ERT within 3 months before study start for subjects aged ≥5 years, and within 6 months before study start for subjects aged <5 years; 3) use of IDS gene therapy at any time; 4) clinically significant thrombocytopenia, other clinically significant coagulation abnormality, or significant active bleeding, or required treatment with an anticoagulant or more than two antiplatelet agents; 5) contraindication for lumbar punctures; 6) have a clinically significant history of stroke, status epilepticus, head trauma with loss of consciousness, or any CNS disease that is not MPS II related within 1 year of screening; 7) have had a ventriculoperitoneal (VP) shunt placed, or any other brain surgery, or have a clinically significant VP shunt malfunction within 30 days of screening; or 8) have any clinically significant CNS trauma or disorder that, in the opinion of the investigator, may interfere with assessment of study endpoints or make participation in the study unsafe.

### Subjects

All subjects who were on a standard-of-care (SOC) treatment (e.g., Elaprase, or idursulfase) prior to initiation of treatment with ETV:IDS were switched to ETV:IDS treatment without a washout period upon enrollment in the study. Anti-drug antibody titer information available for Cohort A indicated that four of the five subjects had anti-drug antibodies (ADAs) against IDS at baseline, with titers ranging from 189 to greater than 11 million. Preliminary information for Cohorts B1 and B2 indicate that for participants who tested positive for ADAs at baseline, titers ranged from 63 to greater than 1 million.

### CGI-C and PGI-C CaGI-C

Subjects in Cohort A (n=5) and a subset of subjects in Cohort B were evaluated using an earlier version (Version 2) of Clinician Global Impression of Change (CGI-C) and Parent/Caregiver Global Impression of Change (PGI-C) instruments through week 24. CGI-C and PGI-C evaluations were performed using a methodology similar to that described in Guy W, editor. ECDEU Assessment Manual for Psychopharmacology. Rockville, MD: US Department of Health, Education, and Welfare Public Health Service Alcohol, Drug Abuse, and Mental Health Administration; 1976. Overall MPSII symptoms, cognitive abilities, behavior, and physical abilities were evaluated. For each evaluation timepoint, change was evaluated relative to the subject's baseline condition at the start of the study. The change in the subject's condition was graded on a 7-point scale: 1=very much improved, 2=much improved, 3=minimally improved, 4=no change, 5=minimally worse, 6=much worse, and 7=very much worse.

In the updated analysis, study participants were evaluated using a current version (Version 3) of Clinician and Caregiver Global Impression of Change instruments (CGI-C and CaGI-C; also referred herein as Clinician and Caregiver Global Impression Scales of Change instruments). Evaluations were performed using a methodology similar to that described in Guy W, editor. ECDEU Assessment Manual for Psychopharmacology. Rockville, MD: US Department of Health, Education, and Welfare Public Health Service Alcohol, Drug Abuse, and Mental Health Administration; 1976. The following category domains were evaluated: Communication, daily living skills, overall MPS II symptoms, problematic behavior, physical abilities, and social skills. For each evaluation timepoint, change was evaluated relative to the subject's baseline condition at the start of the study. The change in the subject's condition was graded on a 5-point scale: 1=much improved, 2=a little improved, 3=no change, 4=a little worse, and 5=much worse.

### Sample Collection

Urine and serum samples were collected at scheduled time-points for GAG (e.g., HS and DS) analysis. In addition, urine samples for total GAG measurement were collected prior to first dose and approximately weekly for the first 13 weeks of study and about every 4 weeks thereafter. Timepoints on the x-axis for the biomarker figures represent intended collection times and may vary by ˜1 week in some patients.

CSF samples were collected at scheduled time-points. CSF samples were collected by lumbar puncture prior to first dose and approximately one week after the 4^{th}, 8^{th}, 12^{th}, 23^{rd} and/or 48^{th} dose of ETV:IDS. In addition, urine samples for total GAG measurement were collected prior to first dose and approximately weekly for the first 13 weeks of study and about every 4 weeks thereafter. Timepoints on the x-axis for the biomarker figures represent intended collection times and may vary by ˜1 week in some patients.

### Quantification of Keratan Sulfate

Total serum KS levels were measured using mass spectrometry and by summing mono-sulfated KS and di-sulfated KS species as previously described (Shimada et al. 2015. JIMD Rep 21:1-13). To determine normal serum KS levels in non-MPS II individuals, KS was measured in commercially available serum samples from 15 normal pediatric subjects (age range of 1 yr-9 yrs), and normal KS was determined to range from about 624 ng/mL to 841 ng/mL (10th to 90th percentile).

### LCMS Assay for GAGs

Quantification of GAGs (e.g., HS and DS) was performed by liquid chromatography using a method similar to that previously described (Pan et al. 2018. Bioanalysis 10(11):825-838; Wang et al. 2018. Biomedical Chromatography 32:e4294 (12 pages)).

### LCMS Assay for BMP and Gangliosides

BMP analyses of CSF were performed by a validated clinical laboratory. Ganglioside and glucosylceramide (GlcCer) analyses of CSF were performed using a method similar to that described in WO 2020/123511.

### Safety Assessments

Safety was evaluated using one or more of the assessments described above, as well as the following: Frequency and severity of adverse events (AEs), including infusion-related reactions (IRRs), which include allergic reactions and anaphylaxis; Vital sign measurements; Physical examinations, including neurological examinations; Safety laboratory assessments (including hematology, serum clinical chemistry, urinalysis, and coagulation); Urine total GAG concentrations (as measured by colorimetric assay and normalized to creatinine); Characterization of immunogenicity of ETV:IDS in serum, as measured by the incidence of anti-drug antibodies (ADAs) during the study relative to baseline; and Use of concomitant medications.

### Statistical Analysis

The statistical analysis is performed using R Software (v. 3.6.2). Continuous variables are summarized using descriptive statistics (number of participants, mean, standard deviation [SD], median, minimum, maximum, and 95% confidence interval [CI] where applicable.) P-values were determined by T-test comparison of mean percent change from baseline.

### Example 2: ETV:IDS Normalizes Keratan Sulfate (KS) Levels in 4.5-Month Old IDS KO×TfR^{muhu} Mice

ETV:IDS 35.23.2 (referred to in Example 2 as ETV:IDS) was administered to a mouse model of MPS II that expresses the chimeric human/mouse transferrin receptor (“IDS KO; TfR^{mu/hu} KI mice”). As described herein, administration of ETV:IDS can normalize serum keratan sulfate (KS) levels in IDS KO; TfR^{mu/hu} KI mice.

### Materials and Methods

### Animal Care and Mouse Strains

Mice were housed under a 12-hour light/dark cycle and had access to water and standard rodent diet (LabDiet® #25502, Irradiated) ad libitum.

A previously described, IDS KO mice on a B6N background were obtained from The Jackson Laboratories (JAX strain 024744). Development and characterization of the TfR^{mu/hu} KI mouse line harboring the human TfR apical domain knocked into the mouse receptor was previously described (U.S. Pat. No. 10,143,187). TfR^{mu/hu} male mice were bred to female IDS heterozygous mice to generate IDS KO×TfR^{mu/hu} mice. All mice used in this study were males and were approximately 4.5 months of age at the start of dosing and 9 months at necropsy.

### Administration and Sample Collection

IDS KO; TfR^{mu/hu} KI mice (n=10) were administered 17 weekly doses of 3 mg/kg of ETV:IDS via intraperitoneal injection. Vehicle-treated TfR^{mu/hu} (n=10) and Ids KO; TfR^{mu/hu} mice (n=10) served as the non-disease and disease comparator group, respectively. All animals were euthanized 7 days following their last dose.

Each animal was observed once daily. Body weights were taken and recorded prior to the first dose and once weekly thereafter, prior to dosing.

In-life and terminal serum samples were collected as follows. Blood was collected via cardiac puncture for serum collection. For serum collection, blood was allowed to clot at room temperature for at least 30 minutes. Tubes were then centrifuged at 12,700 rpm for 7 minutes at 4° C. Serum was transferred to a fresh tube and flash-frozen on dry ice.

### Quantification of Keratan Sulfate

Total serum KS levels were measured using mass spectrometry and by summing mono-sulfated KS and di-sulfated KS species as previously described (Shimada et al. 2015. JIMD Rep 21:1-13).

### Results

As illustrated in FIG. 7, an age-dependent increase in serum KS levels is observed in IDS KO; TfR^{mu/hu} KI mice. Administration of ETV:IDS at 3 mg/kg resulted in normalization of serum KS levels in IDS KO; TfR^{mu/hu} KI mice.

Informal Sequence Listing

| SEQ ID | | |
|---|---|---|
| NO: | Sequence | Description |
| 1 | APELLGGPSVFLFPPKPKDTLMISRTPEVTCVVVDVSHEDPEVKFNWYVD | Wild-type human |
| | GVEVHNAKTKPREEQYNSTYRVVSVLTVLHQDWLNGKEYKCKVSNKALPA | Fc sequence |
| | PIEKTISKAKGQPREPQVYTLPPSRDELTKNQVSLTCLVKGFYPSDIAVE | Positions 231-447 |
| | WESNGQPENNYKTTPPVLDSDGSFFLYSKLTVDKSRWQQGNVFSCSVMHE | EU index |
| | ALHNHYTQKSLSLSPGK | numbering |
| 2 | APELLGGPSVFLFPPKPKDTLMISRTPEVTCVVVDVSHEDPEVKFNWYVD | CH2 domain |
| | GVEVHNAKTKPREEQYNSTYRVVSVLTVLHQDWLNGKEYKCKVSNKALPA | sequence |
| | PIEKTISKAK | Positions 231-340 |
| | | EU index |
| | | numbering |
| 3 | GQPREPQVYTLPPSRDELTKNQVSLTCLVKGFYPSDIAVEWESNGQPENN | CH3 domain |
| | YKTTPPVLDSDGSFFLYSKLTVDKSRWQQGNVFSCSVMHEALHNHYTQKS | sequence |
| | LSLSPGK | Positions 341-447 |
| | | EU index |
| | | numbering |
| 4 | MPPPRTGRGLLWLGLVLSSVCVALGSETQANSTTDALNVLLIIVDDLRPS | Full-length human |
| | LGCYGDKLVRSPNIDQLASHSLLFQNAFAQQAVCAPSRVSFLTGRRPDTT | IDS polypeptide |
| | RLYDFNSYWRVHAGNFSTIPQYFKENGYVTMSVGKVFHPGISSNHTDDSP | sequence |
| | YSWSFPPYHPSSEKYENTKTCRGPDGELHANLLCPVDVLDVPEGTLPDKQ | |
| | STEQAIQLLEKMKTSASPFFLAVGYHKPHIPFRYPKEFQKLYPLENITLA | |
| | PDPEVPDGLPPVAYNPWMDIRQREDVQALNISVPYGPIPVDFQRKIRQSY | |
| | FASVSYLDTQVGRLLSALDDLQLANSTIIAFTSDHGWALGEHGEWAKYSN | |
| | FDVATHVPLIFYVPGRTASLPEAGEKLFPYLDPFDSASQLMEPGRQSMDL | |
| | VELVSLFPTLAGLAGLQVPPRCPVPSFHVELCREGKNLLKHFRFRDLEED | |
| | PYLPGNPRELIAYSQYPRPSDIPQWNSDKPSLKDIKIMGYSIRTIDYRYT | |
| | VWVGFNPDEFLANFSDIHAGELYFVDSDPLQDHNMYNDSQGGDLFQLLMP | |
| 5 | TDALNVLLIIVDDLRPSLGCYGDKLVRSPNIDQLASHSLLFQNAFAQQAV | Mature human IDS |
| | CAPSRVSFLTGRRPDTTRLYDENSYWRVHAGNFSTIPQYFKENGYVTMSV | polypeptide |
| | GKVFHPGISSNHTDDSPYSWSFPPYHPSSEKYENTKTCRGPDGELHANLL | sequence |
| | CPVDVLDVPEGTLPDKQSTEQAIQLLEKMKTSASPFFLAVGYHKPHIPFR | |
| | YPKEFQKLYPLENITLAPDPEVPDGLPPVAYNPWMDIRQREDVQALNISV | |
| | PYGPIPVDFQRKIRQSYFASVSYLDTQVGRLLSALDDLQLANSTIIAFTS | |
| | DHGWALGEHGEWAKYSNFDVATHVPLIFYVPGRTASLPEAGEKLFPYLDP | |
| | FDSASQLMEPGRQSMDLVELVSLFPTLAGLAGLQVPPRCPVPSFHVELCR | |
| | EGKNLLKHFRFRDLEEDPYLPGNPRELIAYSQYPRPSDIPQWNSDKPSLK | |
| | DIKIMGYSIRTIDYRYTVWVGFNPDEFLANFSDIHAGELYFVDSDPLQDH | |
| | NMYNDSQGGDLFQLLMP | |
| 6 | SETQANSTTDALNVLLIIVDDLRPSLGCYGDKLVRSPNIDQLASHSLLFQ | IDS sequence; |
| | NAFAQQAVX₁APSRVSFLTGRRPDTTRLYDENSYWRVHAGNFSTIPQYFKE | X₁, which is |
| | NGYVTMSVGKVFHPGISSNHTDDSPYSWSFPPYHPSSEKYENTKTCRGPD | double |
| | GELHANLLCPVDVLDVPEGTLPDKQSTEQAIQLLEKMKTSASPFFLAVGY | underlined, is |
| | HKPHIPFRYPKEFQKLYPLENITLAPDPEVPDGLPPVAYNPWMDIRQRED | cysteine or |
| | VQALNISVPYGPIPVDFQRKIRQSYFASVSYLDTQVGRLLSALDDLQLAN | formylglycine |
| | STIIAFTSDHGWALGEHGEWAKYSNFDVATHVPLIFYVPGRTASLPEAGE | |
| | KLFPYLDPFDSASQLMEPGRQSMDLVELVSLFPTLAGLAGLQVPPRCPVP | |
| | SFHVELCREGKNLLKHFRFRDLEEDPYLPGNPRELIAYSQYPRPSDIPQW | |
| | NSDKPSLKDIKIMGYSIRTIDYRYTVWVGFNPDEFLANFSDIHAGELYFV | |
| | DSDPLQDHNMYNDSQGGDLFQLLMP | |
| 7 | SETQANSTTDALNVLLIIVDDLRPSLGCYGDKLVRSPNIDQLASHSLLFQ | IDS sequence; |
| | NAFAQQAVCAPSRVSFLTGRRPDTTRLYDENSYWRVHAGNFSTIPQYFKE | cysteine residue |
| | NGYVTMSVGKVFHPGISSNHTDDSPYSWSFPPYHPSSEKYENTKTCRGPD | double underlined |
| | GELHANLLCPVDVLDVPEGTLPDKQSTEQAIQLLEKMKTSASPFFLAVGY | |
| | HKPHIPFRYPKEFQKLYPLENITLAPDPEVPDGLPPVAYNPWMDIRQRED | |
| | VQALNISVPYGPIPVDFQRKIRQSYFASVSYLDTQVGRLLSALDDLQLAN | |
| | STIIAFTSDHGWALGEHGEWAKYSNFDVATHVPLIFYVPGRTASLPEAGE | |
| | KLFPYLDPFDSASQLMEPGRQSMDLVELVSLFPTLAGLAGLQVPPRCPVP | |
| | SFHVELCREGKNLLKHFRFRDLEEDPYLPGNPRELIAYSQYPRPSDIPQW | |
| | NSDKPSLKDIKIMGYSIRTIDYRYTVWVGFNPDEFLANFSDIHAGELYFV | |
| | DSDPLQDHNMYNDSQGGDLFQLLMP | |
| 8 | SETQANSTTDALNVLLIIVDDLRPSLGCYGDKLVRSPNIDQLASHSLLFQ | IDS sequence; |
| | NAFAQQAVfGAPSRVSFLTGRRPDTTRLYDENSYWRVHAGNESTIPQYFK | formylglycine |
| | ENGYVTMSVGKVFHPGISSNHTDDSPYSWSFPPYHPSSEKYENTKTCRGP | residue ″fG″ |
| | DGELHANLLCPVDVLDVPEGTLPDKQSTEQAIQLLEKMKTSASPFFLAVG | double underlined |
| | YHKPHIPFRYPKEFQKLYPLENITLAPDPEVPDGLPPVAYNPWMDIRQRE | |
| | DVQALNISVPYGPIPVDFQRKIRQSYFASVSYLDTQVGRLLSALDDLQLA | |
| | NSTIIAFTSDHGWALGEHGEWAKYSNFDVATHVPLIFYVPGRTASLPEAG | |
| | EKLFPYLDPFDSASQLMEPGRQSMDLVELVSLEPTLAGLAGLQVPPRCPV | |
| | PSFHVELCREGKNLLKHFRFRDLEEDPYLPGNPRELIAYSQYPRPSDIPQ | |
| | WNSDKPSLKDIKIMGYSIRTIDYRYTVWVGFNPDEFLANFSDIHAGELYF | |
| | VDSDPLQDHNMYNDSQGGDLFQLLMP | |
| 9 | EPKSCDKTHTCPPCP | Human IgG1 hinge |
| | | amino acid |
| | | sequence |
| 10 | DKTHTCPPCP | Portion of human |
| | | IgG1 hinge |
| | | sequence |
| 11 | APELLGGPSVFLFPPKPKDTLMISRTPEVTCVVVDVSHEDPEVKFNWYVD | Clone CH3C.35.21 |
| | GVEVHNAKTKPREEQYNSTYRVVSVLTVLHQDWLNGKEYKCKVSNKALPA | |
| | PIEKTISKAKGQPREPQVYTLPPSRDELTKNQVSLTCLVKGFYPSDIAVW | |
| | WESYGTEWSSYKTTPPVLDSDGSFFLYSKLTVTKEEWQQGFVFSCSVMHE | |
| | ALHNHYTQKSLSLSPGK | |
| 12 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17 |
| | PIEKTISKAKGQPREPQVYTLPPSRDELTKNQVSLTCLVKGFYPSDIAVL | |
| | WESYGTEWSSYKTTPPVLDSDGSFFLYSKLTVTKEEWQQGFVFSCSVMHE | |
| | ALHNHYTQKSLSLSPGK | |
| 13 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17.2 |
| | PIEKTISKAKGQPREPQVYTLPPSRDELTKNQVSLTCLVKGFYPSDIAVL | |
| | WESYGTEWASYKTTPPVLDSDGSFFLYSKLTVTKEEWQQGFVFSCSVMHE | |
| | ALHNHYTQKSLSLSPGK | |
| 14 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.2 |
| | PIEKTISKAKGQPREPQVYTLPPSRDELTKNQVSLTCLVKGFYPSDIAVE | |
| | WESYGTEWANYKTTPPVLDSDGSFFLYSKLTVTKEEWQQGFVFSCSVMHE | |
| | ALHNHYTQKSLSLSPGK | |
| 15 | APELLGGPSVFLFPPKPKDTLMISRTPEVTCVVVDVSHEDPEVKFNWYVD | Clone CH3C.35.21 |
| | GVEVHNAKTKPREEQYNSTYRVVSVLTVLHQDWLNGKEYKCKVSNKALPA | with knob |
| | PIEKTISKAKGQPREPQVYTLPPSRDELTKNQVSLWCLVKGFYPSDIAVW | mutation |
| | WESYGTEWSSYKTTPPVLDSDGSFFLYSKLTVTKEEWQQGFVFSCSVMHE | |
| | ALHNHYTQKSLSLSPGK | |
| 16 | APEAAGGPSVFLFPPKPKDTLMISRTPEVTCVVVDVSHEDPEVKFNWYVD | Clone CH3C.35.21 |
| | GVEVHNAKTKPREEQYNSTYRVVSVLTVLHQDWLNGKEYKCKVSNKALPA | with knob and |
| | PIEKTISKAKGQPREPQVYTLPPSRDELTKNQVSLWCLVKGFYPSDIAVW | LALA mutations |
| | WESYGTEWSSYKTTPPVLDSDGSFFLYSKLTVTKEEWQQGFVFSCSVMHE | |
| | ALHNHYTQKSLSLSPGK | |
| 17 | APEAAGGPSVFLFPPKPKDTLMISRTPEVTCVVVDVSHEDPEVKFNWYVD | Clone CH3C.35.21 |
| | GVEVHNAKTKPREEQYNSTYRVVSVLTVLHQDWLNGKEYKCKVSNKALGA | with knob and |
| | PIEKTISKAKGQPREPQVYTLPPSRDELTKNQVSLWCLVKGFYPSDIAVW | LALAPG mutations |
| | WESYGTEWSSYKTTPPVLDSDGSFFLYSKLTVTKEEWQQGFVFSCSVMHE | |
| | ALHNHYTQKSLSLSPGK | |
| 18 | DKTHTCPPCPAPEAAGGPSVFLFPPKPKDTLMISRTPEVTCVVVDVSHED | Clone CH3C.35.21 |
| | PEVKFNWYVDGVEVHNAKTKPREEQYNSTYRVVSVLTVLHQDWLNGKEYK | with knob and |
| | CKVSNKALPAPIEKTISKAKGQPREPQVYTLPPSRDELTKNQVSLWCLVK | LALA mutations |
| | GFYPSDIAVWWESYGTEWSSYKTTPPVLDSDGSFFLYSKLTVTKEEWQQG | and portion of |
| | FVFSCSVMHEALHNHYTQKSLSLSPGK | human IgG1 hinge |
| | | sequence |
| 19 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17 |
| | PIEKTISKAKGQPREPQVYTLPPSRDELTKNQVSLWCLVKGFYPSDIAVL | with knob and |
| | WESYGTEWSSYKTTPPVLDSDGSFFLYSKLTVTKEEWQQGFVFSCSVMHE | LALA mutations |
| | ALHNHYTQKSLSLSPGK | |
| 20 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALGA | CH3C.35.21.17 |
| | PIEKTISKAKGQPREPQVYTLPPSRDELTKNQVSLWCLVKGFYPSDIAVL | with knob and |
| | WESYGTEWSSYKTTPPVLDSDGSFFLYSKLTVTKEEWQQGFVFSCSVMHE | LALAPG mutations |
| | ALHNHYTQKSLSLSPGK | |
| 21 | DKTHTCPPCPAPEAAGGPSVFLFPPKPKDTLMISRTPEVTCVVVDVSHED | Clone |
| | PEVKFNWYVDGVEVHNAKTKPREEQYNSTYRVVSVLTVLHQDWLNGKEYK | CH3C.35.21.17 |
| | CKVSNKALPAPIEKTISKAKGQPREPQVYTLPPSRDELTKNQVSLWCLVK | with knob and |
| | GFYPSDIAVLWESYGTEWSSYKTTPPVLDSDGSFFLYSKLTVTKEEWQQG | LALA mutations |
| | FVFSCSVMHEALHNHYTQKSLSLSPGK | and portion of |
| | | human IgG1 hinge |
| | | sequence |
| 22 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17.2 |
| | PIEKTISKAKGQPREPQVYTLPPSRDELTKNQVSLWCLVKGFYPSDIAVL | with knob |
| | WESYGTEWASYKTTPPVLDSDGSFFLYSKLTVTKEEWQQGFVFSCSVMHE | mutation |
| | ALHNHYTQKSLSLSPGK | |
| 23 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17.2 |
| | PIEKTISKAKGQPREPQVYTLPPSRDELTKNQVSLWCLVKGFYPSDIAVL | with knob and |
| | WESYGTEWASYKTTPPVLDSDGSFFLYSKLTVTKEEWQQGFVFSCSVMHE | LALA mutations |
| | ALHNHYTQKSLSLSPGK | |
| 24 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALGA | CH3C.35.21.17.2 |
| | PIEKTISKAKGQPREPQVYTLPPSRDELTKNQVSLWCLVKGFYPSDIAVL | with knob and |
| | WESYGTEWASYKTTPPVLDSDGSFFLYSKLTVTKEEWQQGFVFSCSVMHE | LALAPG mutations |
| | ALHNHYTQKSLSLSPGK | |
| 25 | DKTHTCPPCPAPEAAGGPSVFLFPPKPKDTLMISRTPEVTCVVVDVSHED | Clone |
| | PEVKFNWYVDGVEVHNAKTKPREEQYNSTYRVVSVLTVLHQDWLNGKEYK | CH3C.35.21.17.2 |
| | CKVSNKALPAPIEKTISKAKGQPREPQVYTLPPSRDELTKNQVSLWCLVK | with knob and |
| | GFYPSDIAVLWESYGTEWASYKTTPPVLDSDGSFFLYSKLTVTKEEWQQG | LALA mutations |
| | FVFSCSVMHEALHNHYTQKSLSLSPGK | and portion of |
| | | human IgG1 hinge |
| | | sequence |
| 26 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.2 with |
| | PIEKTISKAKGQPREPQVYTLPPSRDELTKNQVSLWCLVKGFYPSDIAVE | knob mutation |
| | WESYGTEWANYKTTPPVLDSDGSFFLYSKLTVTKEEWQQGFVFSCSVMHE | |
| | ALHNHYTQKSLSLSPGK | |
| 27 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.2 with |
| | PIEKTISKAKGQPREPQVYTLPPSRDELTKNQVSLWCLVKGFYPSDIAVE | knob and LALA |
| | WESYGTEWANYKTTPPVLDSDGSFFLYSKLTVTKEEWQQGFVFSCSVMHE | mutations |
| | ALHNHYTQKSLSLSPGK | |
| 28 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALGA | CH3C.35.23.2 with |
| | PIEKTISKAKGQPREPQVYTLPPSRDELTKNQVSLWCLVKGFYPSDIAVE | knob and LALAPG |
| | WESYGTEWANYKTTPPVLDSDGSFFLYSKLTVTKEEWQQGFVFSCSVMHE | mutations |
| | ALHNHYTQKSLSLSPGK | |
| 29 | DKTHTCPPCPAPEAAGGPSVFLFPPKPKDTLMISRTPEVTCVVVDVSHED | Clone |
| | PEVKFNWYVDGVEVHNAKTKPREEQYNSTYRVVSVLTVLHQDWLNGKEYK | CH3C.35.23.2 with |
| | CKVSNKALPAPIEKTISKAKGQPREPQVYTLPPSRDELTKNQVSLWCLVK | knob and LALA |
| | GFYPSDIAVEWESYGTEWANYKTTPPVLDSDGSFFLYSKLTVTKEEWQQG | mutations and |
| | FVFSCSVMHEALHNHYTQKSLSLSPGK | portion of human |
| | | IgG1 hinge |
| | | sequence |
| 30 | APELLGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALPA | hole mutations |
| | PIEKTISKAKGQPREPQVYTLPPSRDELTKNQVSLSCAVKGFYPSDIAVE | |
| | WESNGQPENNYKTTPPVLDSDGSFFLVSKLTVDKSRWQQGNVFSCSVMHE | |
| | ALHNHYTQKSLSLSPGK | |
| 31 | APEAAGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALPA | hole and LALA |
| | PIEKTISKAKGQPREPQVYTLPPSRDELTKNQVSLSCAVKGFYPSDIAVE | mutations |
| | WESNGQPENNYKTTPPVLDSDGSFFLVSKLTVDKSRWQQGNVFSCSVMHE | |
| | ALHNHYTQKSLSLSPGK | |
| 32 | SETQANSTTD ALNVLLIIVD DLRPSLGCYG DKLVRSPNID | IDS-Fc fusion |
| | QLASHSLLFQ NAFAQQAVX₁A PSRVSFLTGR RPDTTRLYDF | polypeptide with |
| | NSYWRVHAGN FSTIPQYFKE NGYVTMSVGK VFHPGISSNH | IDS sequence |
| | TDDSPYSWSF PPYHPSSEKY ENTKTCRGPD GELHANLLCP | underlined (X₁, |
| | VDVLDVPEGT LPDKQSTEQA IQLLEKMKTS ASPFFLAVGY | which is double |
| | HKPHIPFRYP KEFQKLYPLE NITLAPDPEV PDGLPPVAYN | underlined, is |
| | PWMDIRQRED VQALNISVPY GPIPVDFQRK IRQSYFASVS | cysteine or |
| | YLDTQVGRLL SALDDLQLAN STIIAFTSDH GWALGEHGEW | formylglycine) |
| | AKYSNFDVAT HVPLIFYVPG RTASLPEAGE KLFPYLDPFD | and hole |
| | SASQLMEPGR QSMDLVELVS LFPTLAGLAG LQVPPRCPVP | mutations |
| | SFHVELCREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APELLGGPSV FLFPPKPKDT | |
| | LMISRTPEVT CVVVDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLSCAVK | |
| | GFYPSDIAVE WESNGQPENN YKTTPPVLDS DGSFFLVSKL | |
| | TVDKSRWQQG NVFSCSVMHE ALHNHYTQKS LSLSPGK | |
| 33 | SETQANSTTD ALNVLLIIVD DLRPSLGCYG DKLVRSPNID | IDS-Fc fusion |
| | QLASHSLLFQ NAFAQQAVCA PSRVSFLTGR RPDTTRLYDF | polypeptide with |
| | NSYWRVHAGN FSTIPQYFKE NGYVTMSVGK VFHPGISSNH | IDS sequence |
| | TDDSPYSWSF PPYHPSSEKY ENTKTCRGPD GELHANLLCP | underlined and |
| | VDVLDVPEGT LPDKQSTEQA IQLLEKMKTS ASPFFLAVGY | hole mutations |
| | HKPHIPFRYP KEFQKLYPLE NITLAPDPEV PDGLPPVAYN | |
| | PWMDIRQRED VQALNISVPY GPIPVDFQRK IRQSYFASVS | |
| | YLDTQVGRLL SALDDLQLAN STIIAFTSDH GWALGEHGEW | |
| | AKYSNFDVAT HVPLIFYVPG RTASLPEAGE KLFPYLDPFD | |
| | SASQLMEPGR QSMDLVELVS LFPTLAGLAG LQVPPRCPVP | |
| | SFHVELCREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APELLGGPSV FLFPPKPKDT | |
| | LMISRTPEVT CVVVDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLSCAVK | |
| | GFYPSDIAVE WESNGQPENN YKTTPPVLDS DGSFFLVSKL | |
| | TVDKSRWQQG NVFSCSVMHE ALHNHYTQKS LSLSPGK | |
| 34 | SETQANSTTD ALNVLLIIVD DLRPSLGCYG DKLVRSPNID | IDS-Fc fusion |
| | QLASHSLLFQ NAFAQQAVfGA PSRVSFLTGR RPDTTRLYDF | polypeptide with |
| | NSYWRVHAGN FSTIPQYFKE NGYVTMSVGK VFHPGISSNH | IDS sequence |
| | TDDSPYSWSF PPYHPSSEKY ENTKTCRGPD GELHANLLCP | underlined |
| | VDVLDVPEGT LPDKQSTEQA IQLLEKMKTS ASPFFLAVGY | (formylglycine |
| | HKPHIPFRYP KEFQKLYPLE NITLAPDPEV PDGLPPVAYN | residue ″fG″ |
| | PWMDIRQRED VQALNISVPY GPIPVDFQRK IRQSYFASVS | double |
| | YLDTQVGRLL SALDDLQLAN STIIAFTSDH GWALGEHGEW | underlined) and |
| | AKYSNFDVAT HVPLIFYVPG RTASLPEAGE KLFPYLDPFD | hole mutations |
| | SASQLMEPGR QSMDLVELVS LFPTLAGLAG LQVPPRCPVP | |
| | SFHVELCREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APELLGGPSV FLFPPKPKDT | |
| | LMISRTPEVT CVVVDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLSCAVK | |
| | GFYPSDIAVE WESNGQPENN YKTTPPVLDS DGSFFLVSKL | |
| | TVDKSRWQQG NVFSCSVMHE ALHNHYTQKS LSLSPGK | |
| 35 | SETQANSTTD ALNVLLIIVD DLRPSLGCYG DKLVRSPNID | IDS-Fc fusion |
| | QLASHSLLFQ NAFAQQAVX₁A PSRVSFLTGR RPDTTRLYDF | polypeptide with |
| | NSYWRVHAGN FSTIPQYFKE NGYVTMSVGK VFHPGISSNH | IDS sequence |
| | TDDSPYSWSF PPYHPSSEKY ENTKTCRGPD GELHANLLCP | underlined (X₁, |
| | VDVLDVPEGT LPDKQSTEQA IQLLEKMKTS ASPFFLAVGY | which is double |
| | HKPHIPFRYP KEFQKLYPLE NITLAPDPEV PDGLPPVAYN | underlined, is |
| | PWMDIRQRED VQALNISVPY GPIPVDFQRK IRQSYFASVS | cysteine or |
| | YLDTQVGRLL SALDDLQLAN STIIAFTSDH GWALGEHGEW | formylglycine) |
| | AKYSNFDVAT HVPLIFYVPG RTASLPEAGE KLFPYLDPED | and hole and LALA |
| | SASQLMEPGR QSMDLVELVS LFPTLAGLAG LQVPPRCPVP | mutations |
| | SFHVELCREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APEAAGGPSV FLFPPKPKDT | |
| | LMISRTPEVT CVVVDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLSCAVK | |
| | GFYPSDIAVE WESNGQPENN YKTTPPVLDS DGSFFLVSKL | |
| | TVDKSRWQQG NVFSCSVMHE ALHNHYTQKS LSLSPGK | |
| 36 | SETQANSTTD ALNVLLIIVD DLRPSLGCYG DKLVRSPNID | IDS-Fc fusion |
| | QLASHSLLFQ NAFAQQAVCA PSRVSFLTGR RPDTTRLYDF | polypeptide with |
| | NSYWRVHAGN FSTIPQYFKE NGYVTMSVGK VFHPGISSNH | IDS sequence |
| | TDDSPYSWSF PPYHPSSEKY ENTKTCRGPD GELHANLLCP | underlined and |
| | VDVLDVPEGT LPDKQSTEQA IQLLEKMKTS ASPFFLAVGY | hole and LALA |
| | HKPHIPFRYP KEFQKLYPLE NITLAPDPEV PDGLPPVAYN | mutations |
| | PWMDIRQRED VQALNISVPY GPIPVDFQRK IRQSYFASVS | |
| | YLDTQVGRLL SALDDLQLAN STIIAFTSDH GWALGEHGEW | |
| | AKYSNEDVAT HVPLIFYVPG RTASLPEAGE KLFPYLDPFD | |
| | SASQLMEPGR QSMDLVELVS LFPTLAGLAG LQVPPRCPVP | |
| | SFHVELCREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLE | |
| | QLLMPGGGGS DKTHTCPPCP APEAAGGPSV FLFPPKPKDT | |
| | LMISRTPEVT CVVVDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLSCAVK | |
| | GFYPSDIAVE WESNGQPENN YKTTPPVLDS DGSFFLVSKL | |
| | TVDKSRWQQG NVFSCSVMHE ALHNHYTQKS LSLSPGK | |
| 37 | SETQANSTTD ALNVLLIIVD DLRPSLGCYG DKLVRSPNID | IDS-Fc fusion |
| | QLASHSLLFQ NAFAQQAVfGA PSRVSFLTGR RPDTTRLYDF | polypeptide with |
| | NSYWRVHAGN FSTIPQYFKE NGYVTMSVGK VFHPGISSNH | IDS sequence |
| | TDDSPYSWSF PPYHPSSEKY ENTKTCRGPD GELHANLLCP | underlined |
| | VDVLDVPEGT LPDKQSTEQA IQLLEKMKTS ASPFFLAVGY | (formylglycine |
| | HKPHIPFRYP KEFQKLYPLE NITLAPDPEV PDGLPPVAYN | residue ″fG″ |
| | PWMDIRQRED VQALNISVPY GPIPVDFQRK IRQSYFASVS | double |
| | YLDTQVGRLL SALDDLQLAN STIIAFTSDH GWALGEHGEW | underlined) and |
| | AKYSNFDVAT HVPLIFYVPG RTASLPEAGE KLFPYLDPED | hole and LALA |
| | SASQLMEPGR QSMDLVELVS LFPTLAGLAG LQVPPRCPVP | mutations |
| | SFHVELCREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APEAAGGPSV FLFPPKPKDT | |
| | LMISRTPEVT CVVVDVSHED PEVKENWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLSCAVK | |
| | GFYPSDIAVE WESNGQPENN YKTTPPVLDS DGSFFLVSKL | |
| | TVDKSRWQQG NVFSCSVMHE ALHNHYTQKS LSLSPGK | |
| 38 | MMDQARSAFSNLFGGEPLSYTRFSLARQVDGDNSHVEMKLAVDEEENADN | Human transferrin |
| | NTKANVTKPKRCSGSICYGTIAVIVFFLIGFMIGYLGYCKGVEPKTECER | receptor protein |
| | LAGTESPVREEPGEDFPAARRLYWDDLKRKLSEKLDSTDFTGTIKLLNEN | 1 (TFR1) |
| | SYVPREAGSQKDENLALYVENQFREFKLSKVWRDQHFVKIQVKDSAQNSV | |
| | IIVDKNGRLVYLVENPGGYVAYSKAATVTGKLVHANFGTKKDFEDLYTPV | |
| | NGSIVIVRAGKITFAEKVANAESLNAIGVLIYMDQTKFPIVNAELSFFGH | |
| | AHLGTGDPYTPGFPSFNHTQFPPSRSSGLPNIPVQTISRAAAEKLFGNME | |
| | GDCPSDWKTDSTCRMVTSESKNVKLTVSNVLKEIKILNIFGVIKGFVEPD | |
| | HYVVVGAQRDAWGPGAAKSGVGTALLLKLAQMFSDMVLKDGFQPSRSIIF | |
| | ASWSAGDFGSVGATEWLEGYLSSLHLKAFTYINLDKAVLGTSNFKVSASP | |
| | LLYTLIEKTMQNVKHPVTGQFLYQDSNWASKVEKLTLDNAAFPFLAYSGI | |
| | PAVSFCFCEDTDYPYLGTTMDTYKELIERIPELNKVARAAAEVAGQFVIK | |
| | LTHDVELNLDYERYNSQLLSFVRDLNQYRADIKEMGLSLQWLYSARGDFF | |
| | RATSRLTTDFGNAEKTDRFVMKKLNDRVMRVEYHELSPYVSPKESPFRHV | |
| | FWGSGSHTLPALLENLKLRKQNNGAFNETLERNQLALATWTIQGAANALS | |
| | GDVWDIDNEF | |
| 39 | NSVIIVDKNGRLVYLVENPGGYVAYSKAATVTGKLVHANFGTKKDFEDLY | Human TfR apical |
| | TPVNGSIVIVRAGKITFAEKVANAESLNAIGVLIYMDQTKFPIVNAELSE | domain |
| | FGHAHLGTGDPYTPGFPSFNHTQFPPSRSSGLPNIPVQTISRAAAEKLFG | |
| | NMEGDCPSDWKTDSTCRMVTSESKNVKLTVS | |
| 40 | GGGGS | Glycine-rich |
| | | linker |
| 41 | GGGGSGGGGS | Glycine-rich |
| | | linker |
| 42 | APELLGGPSVFLFPPKPKDTLMISRTPEVTCVVVDVSHEDPEVKFNWYVD | Wild-type human |
| | GVEVHNAKTKPREEQYNSTYRVVSVLTVLHQDWLNGKEYKCKVSNKALPA | Fc sequence |
| | PIEKTISKAKGQPREPQVYTLPPSRDELTKNQVSLTCLVKGFYPSDIAVE | positions 231-446 |
| | WESNGQPENNYKTTPPVLDSDGSFFLYSKLTVDKSRWQQGNVFSCSVMHE | EU index |
| | ALHNHYTQKSLSLSPG | numbering |
| 43 | APELLGGPSVFLFPPKPKDTLMISRTPEVTCVVVDVSHEDPEVKFNWYVD | Clone CH3C.35.21 |
| | GVEVHNAKTKPREEQYNSTYRVVSVLTVLHQDWLNGKEYKCKVSNKALPA | |
| | PIEKTISKAKGQPREPQVYTLPPSRDELTKNQVSLTCLVKGFYPSDIAVW | |
| | WESYGTEWSSYKTTPPVLDSDGSFFLYSKLTVTKEEWQQGFVFSCSVMHE | |
| | ALHNHYTQKSLSLSPG | |
| 44 | APELLGGPSVFLFPPKPKDTLMISRTPEVTCVVVDVSHEDPEVKFNWYVD | Clone CH3C. 35.21 |
| | GVEVHNAKTKPREEQYNSTYRVVSVLTVLHQDWLNGKEYKCKVSNKALPA | with knob |
| | PIEKTISKAKGQPREPQVYTLPPSRDELTKNQVSLWCLVKGFYPSDIAVW | mutation |
| | WESYGTEWSSYKTTPPVLDSDGSFFLYSKLTVTKEEWQQGFVFSCSVMHE | |
| | ALHNHYTQKSLSLSPG | |
| 45 | APEAAGGPSVFLFPPKPKDTLMISRTPEVTCVVVDVSHEDPEVKFNWYVD | Clone CH3C.35.21 |
| | GVEVHNAKTKPREEQYNSTYRVVSVLTVLHQDWLNGKEYKCKVSNKALPA | with knob and |
| | PIEKTISKAKGQPREPQVYTLPPSRDELTKNQVSLWCLVKGFYPSDIAVW | LALA mutations |
| | WESYGTEWSSYKTTPPVLDSDGSFFLYSKLTVTKEEWQQGFVFSCSVMHE | |
| | ALHNHYTQKSLSLSPG | |
| 46 | APEAAGGPSVFLFPPKPKDTLMISRTPEVTCVVVDVSHEDPEVKFNWYVD | Clone CH3C. 35.21 |
| | GVEVHNAKTKPREEQYNSTYRVVSVLTVLHQDWLNGKEYKCKVSNKALGA | with knob and |
| | PIEKTISKAKGQPREPQVYTLPPSRDELTKNQVSLWCLVKGFYPSDIAVW | LALAPG mutations |
| | WESYGTEWSSYKTTPPVLDSDGSFFLYSKLTVTKEEWQQGFVFSCSVMHE | |
| | ALHNHYTQKSLSLSPG | |
| 47 | DKTHTCPPCPAPEAAGGPSVFLFPPKPKDTLMISRTPEVTCVVVDVSHED | Clone CH3C.35.21 |
| | PEVKFNWYVDGVEVHNAKTKPREEQYNSTYRVVSVLTVLHQDWLNGKEYK | with knob and |
| | CKVSNKALPAPIEKTISKAKGQPREPQVYTLPPSRDELTKNQVSLWCLVK | LALA mutations |
| | GFYPSDIAVWWESYGTEWSSYKTTPPVLDSDGSFFLYSKLTVTKEEWQQG | and portion of |
| | FVFSCSVMHEALHNHYTQKSLSLSPG | human IgG1 hinge |
| | | sequence |
| 48 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17 |
| | PIEKTISKAKGQPREPQVYTLPPSRDELTKNQVSLTCLVKGFYPSDIAVL | |
| | WESYGTEWSSYKTTPPVLDSDGSFFLYSKLTVTKEEWQQGFVFSCSVMHE | |
| | ALHNHYTQKSLSLSPG | |
| 49 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17 |
| | PIEKTISKAKGQPREPQVYTLPPSRDELTKNQVSLWCLVKGFYPSDIAVL | with knob and |
| | WESYGTEWSSYKTTPPVLDSDGSFFLYSKLTVTKEEWQQGFVFSCSVMHE | LALA mutations |
| | ALHNHYTQKSLSLSPG | |
| 50 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALGA | CH3C.35.21.17 |
| | PIEKTISKAKGQPREPQVYTLPPSRDELTKNQVSLWCLVKGFYPSDIAVL | with knob and |
| | WESYGTEWSSYKTTPPVLDSDGSFFLYSKLTVTKEEWQQGFVFSCSVMHE | LALAPG mutations |
| | ALHNHYTQKSLSLSPG | |
| 51 | DKTHTCPPCPAPEAAGGPSVFLFPPKPKDTLMISRTPEVTCVVVDVSHED | Clone |
| | PEVKFNWYVDGVEVHNAKTKPREEQYNSTYRVVSVLTVLHQDWLNGKEYK | CH3C.35.21.17 |
| | CKVSNKALPAPIEKTISKAKGQPREPQVYTLPPSRDELTKNQVSLWCLVK | with knob and |
| | GFYPSDIAVLWESYGTEWSSYKTTPPVLDSDGSFFLYSKLTVTKEEWQQG | LALA mutations |
| | FVFSCSVMHEALHNHYTQKSLSLSPG | and portion of |
| | | human IgG1 hinge |
| | | sequence |
| 52 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.2 |
| | PIEKTISKAKGQPREPQVYTLPPSRDELTKNQVSLTCLVKGFYPSDIAVE | |
| | WESYGTEWANYKTTPPVLDSDGSFFLYSKLTVTKEEWQQGFVFSCSVMHE | |
| | ALHNHYTQKSLSLSPG | |
| 53 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.2 with |
| | PIEKTISKAKGQPREPQVYTLPPSRDELTKNQVSLWCLVKGFYPSDIAVE | knob mutation |
| | WESYGTEWANYKTTPPVLDSDGSFFLYSKLTVTKEEWQQGFVFSCSVMHE | |
| | ALHNHYTQKSLSLSPG | |
| 54 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.23.2 with |
| | PIEKTISKAKGQPREPQVYTLPPSRDELTKNQVSLWCLVKGFYPSDIAVE | knob and LALA |
| | WESYGTEWANYKTTPPVLDSDGSFFLYSKLTVTKEEWQQGFVFSCSVMHE | mutations |
| | ALHNHYTQKSLSLSPG | |
| 55 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALGA | CH3C.35.23.2 with |
| | PIEKTISKAKGQPREPQVYTLPPSRDELTKNQVSLWCLVKGFYPSDIAVE | knob and LALAPG |
| | WESYGTEWANYKTTPPVLDSDGSFFLYSKLTVTKEEWQQGFVFSCSVMHE | mutations |
| | ALHNHYTQKSLSLSPG | |
| 56 | DKTHTCPPCPAPEAAGGPSVFLFPPKPKDTLMISRTPEVTCVVVDVSHED | Clone |
| | PEVKFNWYVDGVEVHNAKTKPREEQYNSTYRVVSVLTVLHQDWLNGKEYK | CH3C.35.23.2 with |
| | CKVSNKALPAPIEKTISKAKGQPREPQVYTLPPSRDELTKNQVSLWCLVK | knob and LALA |
| | GFYPSDIAVEWESYGTEWANYKTTPPVLDSDGSFFLYSKLTVTKEEWQQG | mutations and |
| | FVFSCSVMHEALHNHYTQKSLSLSPG | portion of human |
| | | IgG1 hinge |
| | | sequence |
| 57 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17.2 |
| | PIEKTISKAKGQPREPQVYTLPPSRDELTKNQVSLTCLVKGFYPSDIAVL | |
| | WESYGTEWASYKTTPPVLDSDGSFFLYSKLTVTKEEWQQGFVFSCSVMHE | |
| | ALHNHYTQKSLSLSPG | |
| 58 | APELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17.2 |
| | PIEKTISKAKGQPREPQVYTLPPSRDELTKNQVSLWCLVKGFYPSDIAVL | with knob |
| | WESYGTEWASYKTTPPVLDSDGSFFLYSKLTVTKEEWQQGFVFSCSVMHE | mutation |
| | ALHNHYTQKSLSLSPG | |
| 59 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPA | CH3C.35.21.17.2 |
| | PIEKTISKAKGQPREPQVYTLPPSRDELTKNQVSLWCLVKGFYPSDIAVL | with knob and |
| | WESYGTEWASYKTTPPVLDSDGSFFLYSKLTVTKEEWQQGFVFSCSVMHE | LALA mutations |
| | ALHNHYTQKSLSLSPG | |
| 60 | APEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALGA | CH3C.35.21.17.2 |
| | PIEKTISKAKGQPREPQVYTLPPSRDELTKNQVSLWCLVKGFYPSDIAVL | with knob and |
| | WESYGTEWASYKTTPPVLDSDGSFFLYSKLTVTKEEWQQGFVFSCSVMHE | LALAPG mutations |
| | ALHNHYTQKSLSLSPG | |
| 61 | DKTHTCPPCPAPEAAGGPSVFLFPPKPKDTLMISRTPEVTCVVVDVSHED | Clone |
| | PEVKFNWYVDGVEVHNAKTKPREEQYNSTYRVVSVLTVLHQDWLNGKEYK | CH3C.35.21.17.2 |
| | CKVSNKALPAPIEKTISKAKGQPREPQVYTLPPSRDELTKNQVSLWCLVK | with knob and |
| | GFYPSDIAVLWESYGTEWASYKTTPPVLDSDGSFFLYSKLTVTKEEWQQG | LALA mutations |
| | FVFSCSVMHEALHNHYTQKSLSLSPG | and portion of |
| | | human IgG1 hinge |
| | | sequence |
| 62 | APELLGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALPA | hole mutations |
| | PIEKTISKAKGQPREPQVYTLPPSRDELTKNQVSLSCAVKGFYPSDIAVE | |
| | WESNGQPENNYKTTPPVLDSDGSFFLVSKLTVDKSRWQQGNVFSCSVMHE | |
| | ALHNHYTQKSLSLSPG | |
| 63 | APEAAGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALPA | hole and LALA |
| | PIEKTISKAKGQPREPQVYTLPPSRDELTKNQVSLSCAVKGFYPSDIAVE | mutations |
| | WESNGQPENNYKTTPPVLDSDGSFFLVSKLTVDKSRWQQGNVFSCSVMHE | |
| | ALHNHYTQKSLSLSPG | |
| 64 | SETQANSTTD ALNVLLIIVD DLRPSLGCYG DKLVRSPNID | IDS-Fc fusion |
| | QLASHSLLFQ NAFAQQAVX₁A PSRVSFLTGR RPDTTRLYDF | polypeptide with |
| | NSYWRVHAGN FSTIPQYFKE NGYVTMSVGK VFHPGISSNH | IDS sequence |
| | TDDSPYSWSF PPYHPSSEKY ENTKTCRGPD GELHANLLCP | underlined (X₁, |
| | VDVLDVPEGT LPDKQSTEQA IQLLEKMKTS ASPFFLAVGY | which is double |
| | HKPHIPFRYP KEFQKLYPLE NITLAPDPEV PDGLPPVAYN | underlined, is |
| | PWMDIRQRED VQALNISVPY GPIPVDFQRK IRQSYFASVS | cysteine or |
| | YLDTQVGRLL SALDDLQLAN STIIAFTSDH GWALGEHGEW | formylglycine) |
| | AKYSNFDVAT HVPLIFYVPG RTASLPEAGE KLFPYLDPFD | and hole |
| | SASQLMEPGR QSMDLVELVS LFPTLAGLAG LQVPPRCPVP | mutations |
| | SFHVELCREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APELLGGPSV FLFPPKPKDT | |
| | LMISRTPEVT CVVVDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLSCAVK | |
| | GFYPSDIAVE WESNGQPENN YKTTPPVLDS DGSFFLVSKL | |
| | TVDKSRWQQG NVFSCSVMHE ALHNHYTQKS LSLSPG | |
| 65 | SETQANSTTD ALNVLLIIVD DLRPSLGCYG DKLVRSPNID | IDS-Fc fusion |
| | QLASHSLLFQ NAFAQQAVCA PSRVSFLTGR RPDTTRLYDF | polypeptide with |
| | NSYWRVHAGN FSTIPQYFKE NGYVTMSVGK VFHPGISSNH | IDS sequence |
| | TDDSPYSWSF PPYHPSSEKY ENTKTCRGPD GELHANLLCP | underlined and |
| | VDVLDVPEGT LPDKQSTEQA IQLLEKMKTS ASPFFLAVGY | hole mutations |
| | HKPHIPFRYP KEFQKLYPLE NITLAPDPEV PDGLPPVAYN | |
| | PWMDIRQRED VQALNISVPY GPIPVDFQRK IRQSYFASVS | |
| | YLDTQVGRLL SALDDLQLAN STIIAFTSDH GWALGEHGEW | |
| | AKYSNFDVAT HVPLIFYVPG RTASLPEAGE KLFPYLDPFD | |
| | SASQLMEPGR QSMDLVELVS LFPTLAGLAG LQVPPRCPVP | |
| | SFHVELCREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APELLGGPSV FLFPPKPKDT | |
| | LMISRTPEVT CVVVDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLSCAVK | |
| | GFYPSDIAVE WESNGQPENN YKTTPPVLDS DGSFFLVSKL | |
| | TVDKSRWQQG NVFSCSVMHE ALHNHYTQKS LSLSPG | |
| 66 | SETQANSTTD ALNVLLIIVD DLRPSLGCYG DKLVRSPNID | IDS-Fc fusion |
| | QLASHSLLFQ NAFAQQAVfGA PSRVSELTGR RPDTTRLYDF | polypeptide with |
| | NSYWRVHAGN FSTIPQYFKE NGYVTMSVGK VFHPGISSNH | IDS sequence |
| | TDDSPYSWSF PPYHPSSEKY ENTKTCRGPD GELHANLLCP | underlined |
| | VDVLDVPEGT LPDKQSTEQA IQLLEKMKTS ASPFFLAVGY | (formylglycine |
| | HKPHIPFRYP KEFQKLYPLE NITLAPDPEV PDGLPPVAYN | residue ″fG″ |
| | PWMDIRQRED VQALNISVPY GPIPVDFQRK IRQSYFASVS | double |
| | YLDTQVGRLL SALDDLQLAN STIIAFTSDH GWALGEHGEW | underlined) and |
| | AKYSNFDVAT HVPLIFYVPG RTASLPEAGE KLFPYLDPFD | hole mutations |
| | SASQLMEPGR QSMDLVELVS LFPTLAGLAG LQVPPRCPVP | |
| | SFHVELCREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APELLGGPSV FLFPPKPKDT | |
| | LMISRTPEVT CVVVDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLSCAVK | |
| | GFYPSDIAVE WESNGQPENN YKTTPPVLDS DGSFFLVSKL | |
| | TVDKSRWQQG NVFSCSVMHE ALHNHYTQKS LSLSPG | |
| 67 | SETQANSTTD ALNVLLIIVD DLRPSLGCYG DKLVRSPNID | IDS-Fc fusion |
| | QLASHSLLFQ NAFAQQAVX₁A PSRVSFLTGR RPDTTRLYDF | polypeptide with |
| | NSYWRVHAGN FSTIPQYFKE NGYVTMSVGK VFHPGISSNH | IDS sequence |
| | TDDSPYSWSF PPYHPSSEKY ENTKTCRGPD GELHANLLCP | underlined (X₁, |
| | VDVLDVPEGT LPDKQSTEQA IQLLEKMKTS ASPFFLAVGY | which is double |
| | HKPHIPFRYP KEFQKLYPLE NITLAPDPEV PDGLPPVAYN | underlined, is |
| | PWMDIRQRED VQALNISVPY GPIPVDFQRK IRQSYFASVS | cysteine or |
| | YLDTQVGRLL SALDDLQLAN STIIAFTSDH GWALGEHGEW | formylglycine) |
| | AKYSNFDVAT HVPLIFYVPG RTASLPEAGE KLFPYLDPFD | and hole and LALA |
| | SASQLMEPGR QSMDLVELVS LFPTLAGLAG LQVPPRCPVP | mutations |
| | SFHVELCREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APEAAGGPSV FLFPPKPKDT | |
| | LMISRTPEVT CVVVDVSHED PEVKFNWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLSCAVK | |
| | GFYPSDIAVE WESNGQPENN YKTTPPVLDS DGSFFLVSKL | |
| | TVDKSRWQQG NVFSCSVMHE ALHNHYTQKS LSLSPG | |
| 68 | SETQANSTTD ALNVLLIIVD DLRPSLGCYG DKLVRSPNID | IDS-Fc fusion |
| | QLASHSLLFQ NAFAQQAVCA PSRVSFLTGR RPDTTRLYDF | polypeptide with |
| | NSYWRVHAGN FSTIPQYFKE NGYVTMSVGK VFHPGISSNH | IDS sequence |
| | TDDSPYSWSF PPYHPSSEKY ENTKTCRGPD GELHANLLCP | underlined and |
| | VDVLDVPEGT LPDKQSTEQA IQLLEKMKTS ASPFFLAVGY | hole and LALA |
| | HKPHIPFRYP KEFQKLYPLE NITLAPDPEV PDGLPPVAYN | mutations |
| | PWMDIRQRED VQALNISVPY GPIPVDFQRK IRQSYFASVS | |
| | YLDTQVGRLL SALDDLQLAN STIIAFTSDH GWALGEHGEW | |
| | AKYSNFDVAT HVPLIFYVPG RTASLPEAGE KLFPYLDPFD | |
| | SASQLMEPGR QSMDLVELVS LFPTLAGLAG LQVPPRCPVP | |
| | SFHVELCREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APEAAGGPSV FLFPPKPKDT | |
| | LMISRTPEVT CVVVDVSHED PEVKENWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLSCAVK | |
| | GFYPSDIAVE WESNGQPENN YKTTPPVLDS DGSFFLVSKL | |
| | TVDKSRWQQG NVFSCSVMHE ALHNHYTQKS LSLSPG | |
| 69 | SETQANSTTD ALNVLLIIVD DLRPSLGCYG DKLVRSPNID | IDS-Fc fusion |
| | QLASHSLLFQ NAFAQQAVfGA PSRVSFLTGR RPDTTRLYDF | polypeptide with |
| | NSYWRVHAGN FSTIPQYFKE NGYVTMSVGK VFHPGISSNH | IDS sequence |
| | TDDSPYSWSF PPYHPSSEKY ENTKTCRGPD GELHANLLCP | underlined |
| | VDVLDVPEGT LPDKQSTEQA IQLLEKMKTS ASPFFLAVGY | (formylglycine |
| | HKPHIPFRYP KEFQKLYPLE NITLAPDPEV PDGLPPVAYN | residue ″fG″ |
| | PWMDIRQRED VQALNISVPY GPIPVDFQRK IRQSYFASVS | double |
| | YLDTQVGRLL SALDDLQLAN STIIAFTSDH GWALGEHGEW | underlined) and |
| | AKYSNFDVAT HVPLIFYVPG RTASLPEAGE KLFPYLDPFD | hole and LALA |
| | SASQLMEPGR QSMDLVELVS LFPTLAGLAG LQVPPRCPVP | mutations |
| | SFHVELCREG KNLLKHFRFR DLEEDPYLPG NPRELIAYSQ | |
| | YPRPSDIPQW NSDKPSLKDI KIMGYSIRTI DYRYTVWVGF | |
| | NPDEFLANFS DIHAGELYFV DSDPLQDHNM YNDSQGGDLF | |
| | QLLMPGGGGS DKTHTCPPCP APEAAGGPSV FLFPPKPKDT | |
| | LMISRTPEVT CVVVDVSHED PEVKENWYVD GVEVHNAKTK | |
| | PREEQYNSTY RVVSVLTVLH QDWLNGKEYK CKVSNKALPA | |
| | PIEKTISKAK GQPREPQVYT LPPSRDELTK NQVSLSCAVK | |
| | GFYPSDIAVE WESNGQPENN YKTTPPVLDS DGSFFLVSKL | |
| | TVDKSRWQQG NVFSCSVMHE ALHNHYTQKS LSLSPG | |

All publications, patents, and patent documents are incorporated by reference herein, as though individually incorporated by reference. The present disclosure has been described with reference to various specific and preferred embodiments and techniques. However, it should be understood that many variations and modifications may be made while remaining within the spirit and scope of the invention.

What is claimed is:

1. A method of providing added peripheral iduronate 2-sulfatase (IDS) activity relative to a standard-of-care treatment to a subject with Hunter syndrome, comprising administering to the subject a therapeutically effective dose of a pharmaceutical composition comprising a protein, wherein the administration of the pharmaceutical composition reduces serum levels of keratan sulfate (KS) in the subject relative to a baseline level measured in the subject prior to the administration, and wherein the protein comprises an IDS amino acid sequence and a transferrin receptor (TfR) binding domain.

2. A method of providing added peripheral iduronate 2-sulfatase (IDS) activity relative to a standard-of-care treatment to a subject with Hunter syndrome, comprising administering to the subject a therapeutically effective dose of a pharmaceutical composition comprising a protein, wherein the administration of the pharmaceutical composition reduces serum levels of keratan sulfate (KS) in the subject to a baseline level measured in a healthy subject or in a subject that does not have Hunter syndrome, and wherein the protein comprises an IDS amino acid sequence and a transferrin receptor (TfR) binding domain.

3. A method of reducing and/or normalizing serum keratan sulfate (KS) levels in a subject with Hunter syndrome, comprising administering to the subject a therapeutically effective dose of a pharmaceutical composition comprising a protein, wherein the protein comprises an iduronate 2-sulfatase (IDS) amino acid sequence and a transferrin receptor (TfR) binding domain.

4. A method for evaluating peripheral iduronate 2-sulfatase (IDS) activity in a subject having Hunter syndrome, comprising:
(a) determining a first marker profile by detecting the level of keratan sulfate (KS) in a sample from the subject obtained at baseline or prior to initiation of treatment with a pharmaceutical composition comprising a protein;
(b) administering to the subject a therapeutically effective dose of the pharmaceutical composition comprising the protein;
(c) determining a second marker profile by detecting the level of KS in a sample from the subject obtained at a time point after administration of the pharmaceutical composition comprising the protein; and
(d) comparing the first and the second marker profiles;
wherein a decrease in KS levels between the first and the second marker profiles indicates added peripheral IDS activity in the subject, and
wherein the protein comprises an IDS amino acid sequence and a transferrin receptor (TfR) binding domain.

5. The method of any one of claims 1-4, wherein the subject's serum heparan sulfate (HS) and/or dermatan sulfate (DS) levels decrease after administration of the pharmaceutical composition.

6. The method of any one of claims 1-5, wherein the subject's urine heparan sulfate (HS) and/or dermatan sulfate (DS) levels decrease after administration of the pharmaceutical composition.

7. The method of any one of claims 1-6, wherein the subject's total urine GAG levels decrease after administration of the pharmaceutical composition.

8. The method of any one of claims 1-7, wherein the pharmaceutical composition is administered weekly.

9. The method of claim 8, wherein the reduction and/or normalization of KS is sustained after at least 12 weekly doses.

10. The method of claim 8, wherein the reduction and/or normalization of KS is sustained after at least 23 weekly doses.

11. The method of any one of claims 8-10, wherein the subject's serum heparan sulfate (HS) and/or dermatan sulfate (DS) levels decrease and/or normalize relative to a baseline level by at least 12 weekly doses of the pharmaceutical composition.

12. The method of any one of claims 8-10, wherein the subject's serum heparan sulfate (HS) and/or dermatan sulfate (DS) levels decrease and/or normalize relative to a baseline level by at least 23 weekly doses of the pharmaceutical composition.

13. The method of any one of claims 8-10, wherein the subject's serum heparan sulfate (HS) and/or dermatan sulfate (DS) levels decrease and/or normalize relative to a baseline level by at least 48 weekly doses of the pharmaceutical composition.

14. The method of any one of claims 8-13, wherein the subject's urine heparan sulfate (HS) and/or dermatan sulfate (DS) levels decrease and/or normalize relative to a baseline level after by at least 12 weekly doses of the pharmaceutical composition.

15. The method of any one of claims 8-13, wherein the subject's urine heparan sulfate (HS) and/or dermatan sulfate (DS) levels decrease and/or normalize relative to a baseline level after by at least 23 weekly doses of the pharmaceutical composition.

16. The method of any one of claims 8-13, wherein the subject's urine heparan sulfate (HS) and/or dermatan sulfate (DS) levels decrease and/or normalize relative to a baseline level after by at least 48 weekly doses of the pharmaceutical composition.

17. The method of any one of claims 8-13, wherein the subject's total urine GAG levels decrease relative to a baseline level after at least 12 weekly doses of the pharmaceutical composition.

18. The method of any one of claims 8-13, wherein the subject's total urine GAG levels decrease relative to a baseline level after at least 23 weekly doses of the pharmaceutical composition.

19. The method of any one of claims 8-13, wherein the subject's total urine GAG levels decrease relative to a baseline level after at least 48 weekly doses of the pharmaceutical composition.

20. A method of providing added peripheral iduronate 2-sulfatase (IDS) activity relative to a standard-of-care treatment to a subject with Hunter syndrome, comprising administering to the subject a therapeutically effective dose of a pharmaceutical composition comprising a protein, wherein the administration of the pharmaceutical composition normalizes levels of urine heparan sulfate (HS) and/or dermatan sulfate (DS) in the subject by at least 12 weekly doses, and wherein normalization is sustained after at least 23 weekly doses, and wherein the protein comprises an IDS amino acid sequence and a transferrin receptor (TfR) binding domain.

21. A method of providing added peripheral iduronate 2-sulfatase (IDS) activity relative to a standard-of-care treatment to a subject with Hunter syndrome, comprising administering to the subject at least 23 weekly therapeutically effective doses of a pharmaceutical composition comprising a protein, wherein the administration of the pharmaceutical composition normalizes levels of urine heparan sulfate (HS) and/or dermatan sulfate (DS) in the subject by at least 12 weekly doses, and wherein normalization is sustained after at least 23 weekly doses, and wherein the protein comprises an IDS amino acid sequence linked to an Fc polypeptide; and a transferrin receptor (TfR) binding domain.

22. A method of normalizing urine heparan sulfate (HS) and/or dermatan sulfate (DS) levels in a subject with Hunter syndrome, comprising administering to the subject a therapeutically effective dose of a pharmaceutical composition comprising a protein, wherein the HS and/or DS levels in the subject are normalized by at least 12 weekly doses, wherein normalization is sustained after at least 23 weekly doses, and wherein the protein comprises an IDS amino acid sequence and a transferrin receptor (TfR) binding domain.

23. A method of normalizing urine heparan sulfate (HS) and/or dermatan sulfate (DS) levels in a subject with Hunter syndrome, comprising administering to the subject at least 23 weekly therapeutically effective doses of a pharmaceutical composition comprising a protein, wherein the HS and/or DS levels in the subject are normalized by at least 12 weekly doses, wherein normalization is sustained after at least 23 weekly doses, and wherein the protein comprises an IDS amino acid sequence and a transferrin receptor (TfR) binding domain.

24. The method of any one of claims 20-23, wherein the normalization of HS and/or DS is sustained after at least 30 weekly doses.

25. The method of any one of claims 20-23, wherein the normalization of HS and/or DS is sustained after at least 36 weekly doses.

26. The method of any one of claims 20-23, wherein the normalization of HS and/or DS is sustained after at least 42 weekly doses.

27. The method of any one of claims 20-23, wherein the normalization of HS and/or DS is sustained after at least 48 weekly doses.

28. The method of any one of claims 20-23, wherein the subject's total urine GAG levels decrease relative to a baseline level measured prior to administration after at least 12 weekly doses.

29. The method of any one of claims 20-23, wherein the subject's total urine GAG levels decrease relative to a baseline level measured prior to administration after at least 23 weekly doses.

30. The method of any one of claims 20-23, wherein the subject's total urine GAG levels decrease relative to a baseline level measured prior to administration after at least 48 weekly doses.

31. The method of any one of claims 1-30, wherein the protein comprises an IDS amino acid sequence linked to an Fc polypeptide; and a transferrin receptor (TfR) binding domain.

32. The method of claim 31, wherein the protein comprises a fusion polypeptide comprising a first Fc polypeptide linked to the IDS amino acid sequence; a second Fc polypeptide that forms an Fc dimer with the first Fc polypeptide; and a transferrin receptor (TfR) binding domain.

33. The method of any one of claims 1-32, wherein the TfR binding domain is comprised within an Fc polypeptide.

34. The method of any one of claims 1-33, wherein the protein does not comprise an immunoglobulin heavy and/or light chain variable region sequence or an antigen-binding portion thereof.

35. The method of any one of claims 1-34, wherein the protein comprises:
(a) a fusion polypeptide comprising a first Fc polypeptide linked to an iduronate 2-sulfatase (IDS) amino acid sequence, wherein the fusion polypeptide comprises SEQ ID NO: 35, 36, 37, 67, 68 or 69; and
(b) a second Fc polypeptide comprising SEQ ID NO: 29 or 56.

36. A method of providing a clinical benefit to a subject with Hunter syndrome, comprising administering to the subject a therapeutically effective dose of a pharmaceutical composition comprising a protein, wherein the administration of the pharmaceutical composition produces an improvement in a clinical outcome as measured by one or more clinical Global Impression of Change instruments relative to baseline levels measured in the subject prior to the administration, and wherein the protein comprises:
a) a fusion polypeptide comprising a first Fc polypeptide linked to an iduronate 2-sulfatase (IDS) amino acid sequence, wherein the fusion polypeptide comprises SEQ ID NO:35, 36, 37, 67, 68, or 69; and
b) a second Fc polypeptide comprising SEQ ID NO:29 or 56.

37. The method of claim 36, wherein the one or more clinical Global Impression of Change instruments are Clinician Global Impression of Change (CGI-C) and/or Parent/Caregiver Global Impression of Change (PGI-C); or wherein the one or more clinical Global Impression of Change instruments are Clinician Global Impression of Change (CGI-C) and/or Caregiver Global Impression of Change (CaGI-C).

38. The method of claim 37, wherein the administration of the pharmaceutical composition produces an improvement in the subject's overall Hunter syndrome symptoms, as measured by the CGI-C and/or PGI-C or CaGI-C.

39. The method of claim 37 or 38, wherein the administration of the pharmaceutical composition produces an improvement in the subject's cognitive abilities and/or behavior, as measured by the CGI-C and/or PGI-C.

40. The method of any one of claims 37-39 wherein the administration of the pharmaceutical composition produces an improvement in the subject's communication, daily living skills, problematic behavior and/or social skills, as measured by the CGI-C and/or CaGI-C.

41. The method of any one of claims 37-40, wherein the administration of the pharmaceutical composition produces an improvement in the subject's physical abilities, as measured by the CGI-C and/or PGI-C or CaGI-C.

42. The method of any one of claims 37-41, wherein the clinical outcome is minimally improved, as measured by the CGI-C and/or PGI-C; or wherein the clinical outcome is a little improved as measured by the CGI-C and/or CaGI-C.

43. The method of any one of claims 37-41, wherein the clinical outcome is much improved, as measured by the CGI-C and/or PGI-C or CaGI-C.

44. The method of any one of claims 37-41, wherein the clinical outcome is very much improved, as measured by the CGI-C and/or PGI-C.

45. The method of any one of claims 37-44, wherein the improvement is measured by the CGI-C.

46. The method of any one of claims 37-44, wherein the improvement is measured by the PGI-C or CaGI-C.

47. The method of any one of claims 1-46, wherein the therapeutically effective dose is from about 3 mg/kg to about 30 mg/kg of protein.

48. The method of claim 47, wherein the therapeutically effective dose is about 3 mg/kg of protein.

49. The method of claim 47, wherein the therapeutically effective dose is about 7.5 mg/kg of protein.

50. The method of claim 47, wherein the therapeutically effective dose is about 15 mg/kg of protein.

51. The method of claim 47, wherein the therapeutically effective dose is about 30 mg/kg of protein.

52. A method of improving treatment in a patient having non-neuronopathic Hunter syndrome, comprising switching the patient from idursulfase enzyme replacement therapy to treatment with a pharmaceutical composition comprising a protein, wherein the protein comprises:
a) a fusion polypeptide comprising a first Fc polypeptide linked to an iduronate 2-sulfatase (IDS) amino acid sequence, wherein the fusion polypeptide comprises SEQ ID NO:35, 36, 37, 67, 68, or 69; and
b) a second Fc polypeptide comprising SEQ ID NO:29 or 56.

53. The method of claim 52, wherein switching the patient from idursulfase enzyme replacement therapy to treatment with the pharmaceutical composition reduces the level of a glycosaminoglycan (GAG) in the cerebrospinal fluid (CSF) of the patient relative to a baseline level in the patient measured before the switching.

54. The method of claim 53, wherein the level of the GAG in the CSF of the patient is reduced to a level measured in a healthy subject or a subject that does not have Hunter Syndrome.

55. The method of claim 53 or 54, wherein the GAG is heparan sulfate.

56. The method of claim 53 or 54, wherein the GAG is dermatan sulfate.

57. The method of any one of claims 52-56, wherein switching the patient from idursulfase enzyme replacement therapy to treatment with the pharmaceutical composition reduces the total level of glycosaminoglycans (GAGs) in the urine of the patient relative to a baseline level measured in the patient before the switching, thereby providing added peripheral activity.

58. The method of any one of claims 52-57, wherein the treatment with the pharmaceutical composition comprises administering a dose of from about 3 mg/kg to about 30 mg/kg of protein.

59. The method of claim 58, wherein the dose is about 3 mg/kg of protein.

60. The method of claim 58, wherein the dose is about 7.5 mg/kg of protein.

61. The method of claim 58, wherein the dose is about 15 mg/kg of protein.

62. The method of claim 58, wherein the dose is about 30 mg/kg of protein.

63. The method of any one of claims 1-62, wherein the pharmaceutical composition is administered intravenously.

64. A method of resolving an infusion-related reaction (IRR) in a subject receiving treatment for Hunter syndrome, wherein the subject is being intravenously administered or was intravenously administered an initial dose of a pharmaceutical composition comprising a protein, the method comprising reducing the amount and/or the infusion rate of administration of a subsequent dose of the pharmaceutical composition, wherein the protein comprises:
a) a fusion polypeptide comprising a first Fc polypeptide linked to an iduronate 2-sulfatase (IDS) amino acid sequence, wherein the fusion polypeptide comprises SEQ ID NO:35, 36, 37, 67, 68, or 69; and
b) a second Fc polypeptide comprising SEQ ID NO:29 or 56.

65. The method of claim 64, wherein the method comprises reducing the amount of the subsequent dose of the pharmaceutical composition.

66. The method of claim 64, wherein the method comprises reducing the infusion rate of administration of the subsequent dose of the pharmaceutical composition.

67. The method of claim 64, wherein the method comprises reducing the amount and the infusion rate of administration of the subsequent dose of the pharmaceutical composition.

68. A method of resolving an IRR in a subject receiving treatment for Hunter syndrome, comprising modifying the subject's treatment regimen, wherein the treatment regimen comprises intravenous administration of an initial dose of a pharmaceutical composition comprising a protein, and the modified regimen comprises administering a subsequent dose of the pharmaceutical composition to the subject, wherein the subsequent dose is a reduced dose and/or is administered at a reduced rate of infusion, and wherein the protein comprises:
a) a fusion polypeptide comprising a first Fc polypeptide linked to an iduronate 2-sulfatase (IDS) amino acid sequence, wherein the fusion polypeptide comprises SEQ ID NO:35, 36, 37, 67, 68, or 69; and
b) a second Fc polypeptide comprising SEQ ID NO:29 or 56.

69. The method of claim 68, wherein the subsequent dose is a reduced dose.

70. The method of claim 68, wherein the subsequent dose is administered at a reduced rate of infusion.

71. The method of claim 68, wherein the subsequent dose is a reduced dose and is administered at a reduced rate of infusion.

72. The method of any one of claims 64-71, wherein the IRR is anaphylaxis.

73. The method of claim 72, wherein the anaphylaxis meets the Sampson criteria.

74. The method of any one of claims 64-73, wherein the method further comprises administering to the subject one or more therapeutic agents useful for treating an IRR, wherein the one or more agents are administered prior to the administration of the subsequent dose, co-administered with the subsequent dose, or are administered after the subsequent dose.

75. The method of claim 74, wherein the one or more therapeutic agents are administered prior to the administration of the subsequent dose.

76. The method of claim 74 or 75, wherein the one or more therapeutic agents are selected from the group consisting of: an anti-histamine, an anti-pyretic, a corticosteroid, and combinations thereof.

77. The method of claim 76, wherein the one or more therapeutic agents comprise a combination of acetaminophen and diphenhydramine.

78. The method of any one of claims 64-77, wherein the initial dose is from about 3 mg/kg to about 30 mg/kg of protein.

79. The method of claim 78, wherein the initial dose is about 3 mg/kg of protein.

80. The method of claim 78, wherein the initial dose is about 7.5 mg/kg of protein.

81. The method of claim 78, wherein the initial dose is about 15 mg/kg of protein.

82. The method of claim 78, wherein the initial dose is about 30 mg/kg of protein.

83. The method of any one of claims 1-82, wherein the subject or patient is a human subject or patient.

84. The method of claim 83, wherein the subject or patient is a human male subject or patient.

85. The method of any one of claims 1-84, wherein the subject or patient has neuronopathic Hunter syndrome (nMPS II) or has a same genetic mutation in the IDS gene as a blood relative with confirmed nMPS II.

86. The method of any one of claims 1-85, wherein the subject or patient had previously received idursulfase enzyme replacement therapy and switched to the administration of the pharmaceutical composition.

87. The method of claim 86, wherein the subject or patient was switched from administration of idursulfase enzyme replacement therapy to administration of the pharmaceutical composition without a washout period.

88. The method of any one of claims 1-87, wherein the subject or patient had pre-existing anti-drug antibodies against IDS prior to administration of the pharmaceutical composition.

89. The method of claim 88, wherein the subject's or patient's titer of anti-drug antibodies against IDS ranges from 189 to greater than 11 million.

90. The method of claim 89, wherein the subject's or patient's titer of anti-drug antibodies against IDS is greater than 11 million.

91. The method of any one of claims 36-90, wherein the pharmaceutical composition is administered weekly.

92. The method of claim 91, wherein the pharmaceutical composition is administered weekly for at least 12 weeks.

93. The method of claim 91, wherein the pharmaceutical composition is administered weekly for at least 23 weeks.

94. The method of any one of claims 1-93, wherein the pharmaceutical composition further comprises a pharmaceutically acceptable excipient.

95. The method of any one of claims 35-94, wherein the fusion polypeptide comprises SEQ ID NO: 35; and the second Fc polypeptide comprises SEQ ID NO: 29.

96. The method of any one of claims 35-94, wherein the fusion polypeptide is SEQ ID NO: 35; and the second Fc polypeptide is SEQ ID NO: 29.

97. The method of any one of claims 35-94, wherein the fusion polypeptide comprises SEQ ID NO: 67; and the second Fc polypeptide comprises SEQ ID NO: 56.

98. The method of any one of claims 35-94, wherein the fusion polypeptide is SEQ ID NO: 67; and the second Fc polypeptide is SEQ ID NO: 56.

99. The method of any one of claims 35-94, wherein the fusion polypeptide comprises SEQ ID NO: 37; and the second Fc polypeptide comprises SEQ ID NO: 29.

100. The method of any one of claims 35-94, wherein the fusion polypeptide is SEQ ID NO: 37; and the second Fc polypeptide is SEQ ID NO: 29.

101. The method of any one of claims 35-94, wherein the fusion polypeptide comprises SEQ ID NO: 69; and the second Fc polypeptide comprises SEQ ID NO: 56.

102. The method of any one of claims 35-94, wherein the fusion polypeptide is SEQ ID NO: 69; and the second Fc polypeptide is SEQ ID NO: 56.
